(19) United States
(12) Patent Application Publication
(10) Pub. No.: US 2016/0222056 A1
(43) Pub. Date: Aug. 4, 2016

(54) SUBSTITUTED PHENYLALANINE DERIVATIVES

(71) Applicant: BAYER PHARMA AKTIENGESELLSCHAFT, Berlin (DE)

(72) Inventors: Ulrike RÖHN, Berlin (DE); Manuel ELLERMANN, Berlin (DE); Julia STRASSBURGER, Wuppertal (DE); Astrid WENDT, Syke (DE); Susanne RÖHRIG, Hilden (DE); Robert Alan WEBSTER, Wuppertal (DE); Martina Victoria SCHMIDT, Köln (DE); Adrian TERSTEEGEN, Wuppertal (DE); Kristin BEYER, Kensington, CA (US); Martina SCHÄFER, Berlin (DE); Anja BUCHMÜLLER, Essen (DE); Christoph GERDES, Köln (DE); Michael SPERZEL, Kierspe (DE); Steffen SANDMANN, Essen-Heisingen (DE); Stefan HEITMEIER, Wülfrath (DE); Alexander HILLISCH, Solingen (DE); Jens ACKERSTAFF, Düsseldorf (DE); Carsten TERJUNG, Bochum (DE)

(73) Assignee: Bayer Pharma Aktiengesellschaft, Berlin (DE)

(21) Appl. No.: 15/024,974

(22) PCT Filed: Sep. 24, 2014
(86) PCT No.: PCT/EP2014/070303

§ 371 Date: Mar. 25, 2016

(30) Foreign Application Priority Data
Sep. 26, 2013 (EP) 13186056.1

(51) Int. Cl. C07K 5/065

(52) U.S. Cl. CPC C07K 5/06078

(57) ABSTRACT

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

The invention relates to substituted phenylalanine derivatives and to processes for preparation thereof, and to the use thereof for production of medicaments for treatment and/or prophylaxis of diseases, especially of cardiovascular disorders and/or severe perioperative blood loss.

Blood coagulation is a protective mechanism of the organism which helps to “seal” defects in the wall of the blood vessels quickly and reliably. Thus, loss of blood can be avoided or kept to a minimum. Haemostasis after injury of the blood vessels is effected mainly by the coagulation system in which an enzymatic cascade of complex reactions of plasma proteins is triggered. Numerous blood coagulation factors are involved in this process, each of which factors converts, on activation, the respectively next inactive precursor into its active form. At the end of the cascade comes the conversion of soluble fibrinogen into insoluble fibrin, resulting in the formation of a blood clot. In blood coagulation, traditionally the intrinsic and the extrinsic system, which end in a final joint reaction path, are distinguished. Here, factors Xa and IIa (thrombin) play key roles: Factor Xa bundles the signals of the two coagulation paths since it is formed both via factor VIIa/tissue factor (extrinsic path) and via the tenase complex (intrinsic path) by conversion of factor X. The activated serine protease Xa cleaves prothrombin to thrombin which, via a series of reactions, transduces the impulses from the cascade to the coagulation state of the blood.

In the more recent past, the traditional theory of two separate regions of the coagulation cascade (extrinsic and intrinsic path) has been modified owing to new findings: In these models, coagulation is initiated by binding of activated factor VIIa to tissue factor (TF). The resulting complex activates factor X, which in turn leads to generation of thrombin with subsequent production of fibrin and platelet activation (via PAR-1) as injury-sealing end products of haemostasis. Compared to the subsequent amplification/propagation phase, the thrombin production rate is low and as a result of the occurrence of TFPI as inhibitor of the TF-FVIIa-FX complex is limited in time.

A central component of the transition from initiation to amplification and propagation of coagulation is factor XIa. In positive feedback loops, thrombin activates, in addition to factor V and factor VIII, also factor XI to factor XIa, whereby factor IX is converted into factor IXa, thus, via the factor IXa/factor VIIIa complex generated in this manner, rapidly producing relatively large amounts of factor Xa. This triggers the production of large amounts of thrombin, leading to strong thrombus growth and stabilizing the thrombus.

The formation of a thrombus or blood clot is counter-regulated by fibrinolysis. Activation of plasminogen by tissue plasminogen activator (tPA) results in formation of the active serine protease, plasmin, which cleaves polymerized fibrin and thus forms the thrombus. This process is referred to as fibrinolysis—with plasmin as key enzyme.

Uncontrolled activation of the coagulation system or defects in the inhibition of the activation processes may cause formation of local thromboses or embolisms in vessels (arteries, veins, lymph vessels) or heart chambers. This may lead to serious thrombotic or thromboembolic disorders. In addition, systemic hypercoagulability may lead to consumption coagulopathy in the context of a disseminated intravasal coagulation.

In the course of many cardiovascular and metabolic disorders, there is an increased tendency for coagulation and platelet activation owing to systemic factors such as hyperlipidaemia, diabetes or smoking, owing to changes in blood flow with stasis, for example in atrial fibrillation, or owing to pathological changes in vessel walls, for example endothelial dysfunctions or atherosclerosis. This unwanted and excessive haemostasis may, by formation of fibrin- and platelet-rich thrombi, lead to thromboembolic disorders and thrombotic complications with life-threatening conditions.

Thromboembolic disorders are the most frequent cause of morbidity and mortality in most industrialized countries [Heart Disease: A Textbook of Cardiovascular Medicine, Eugene Braunwald, 5th edition, 1997, W.B. Saunders Company, Philadelphia].

The anticoagulants known from the prior art, for example substances for inhibiting or preventing blood coagulation, have various, frequently grave disadvantages. Accordingly, in practice, efficient treatment methods or the prophylaxis of thrombotic/thromboembolic disorders is frequently found to be very difficult and unsatisfactory.

In the therapy and prophylaxis of thromboembolic disorders, use is made, firstly, of heparin which is administered parenterally or subcutaneously. Because of more favourable pharmacokinetic properties, preference is these days increasingly given to low-molecular-weight heparin; however, the known disadvantages described hereinbelow encountered in heparin therapy cannot be avoided either in this manner. Thus, heparin is orally ineffective and has only a comparatively short half-life. In addition, there is a high risk of bleeding, there may in particular be cerebral haemorrhages and bleeding in the gastrointestinal tract, and there may be thrombopaenia, alopecia medicomentosa or osteoporosis [Pschyrembel, Klinisches Wörterbuch [clinical dictionary], 257th edition, 1994, Walter de Gruyter Verlag, page 610, keyword “Heparin”; Römpp Lexikon Chemie, version 1.5, 1998, Georg Thieme Verlag Stuttgart, keyword “Heparin”]. Low-molecular-weight heparins do have a lower probability of leading to the development of heparin-induced thrombocytopaenia; however, they can likewise only be administered subcutaneously. This also applies to fondaparinux, a synthetically produced selective factor Xa inhibitor having a long half-life.

A second class of anticoagulants are the vitamin K antagonists. These include, for example, 1,3-indanediones and in particular compounds such as warfarin, phenprocoumon, dicumarol and other coumarin derivatives which non-selectively inhibit the synthesis of various products of certain vitamin K-dependent coagulation factors in the liver. Owing to the mechanism of action, the onset of action is very slow (latency to the onset of action 36 to 48 hours). The compounds can be administered orally; however, owing to the high risk of bleeding and the narrow therapeutic index complicated individual adjustment and monitoring of the patient are required [J. Hirsh, J. Dalen, D. R. Anderson et al., “Oral anticoagulants: Mechanism of action, clinical effectiveness, and optimal therapeutic range” Chest 2001, 119, 8S-21S; J. Ansell, J. Hirsh, J. Dalen et al., “Managing oral anticoagulant therapy” Chest 2001, 119, 22S-38S; P. S. Wells, A. M. Holbrook, N. R. Crowther et al., “Interactions of warfarin with drugs and food” Ann. Intern. Med. 1994, 121, 676-683]. In addition, other side-effects such as gastrointestinal problems, hair loss and skin necroses have been described.

More recent approaches for oral anticoagulants are in various phases of clinical evaluation or in clinical use, but they have also shown disadvantages, for example highly variable bioavailability, liver damage and bleeding complications.

For antithrombotic medicaments, the therapeutic width is of central importance: The distance between the therapeutically active dose for coagulation inhibition and the dose where bleeding may occur should be as big as possible so that maximum therapeutic activity is achieved at a minimum risk profile.

In various in vivo models with, for example, antibodies as factor XIa inhibitors, but also in factor XIa knock-out models, the antithrombotic effect with small/no prolongation of bleeding time or extension of blood volume was confirmed. In clinical studies, elevated factor XIa concentrations were associated with an increased event rate. However, factor XI deficiency (haemophilia C), in contrast to factor VIIIa or factor IXa (haemophilia A and B, respectively), did not lead to spontaneous bleeding and was only noticed during surgical interventions and traumata. Instead, protection against certain thromboembolic events was found.

In the event of hyperfibrinolytic states, there is inadequate wound closure, which causes severe, sometimes life-threatening, bleeding. This bleeding can be stopped by the inhibition of fibrinolysis with antifibrinolytics, by which plasmin activity is reduced. Corresponding effects with the plasminogen inhibitor tranexamic acid have been shown in various clinical studies.

It is therefore an object of the present invention to provide novel compounds for treatment and/or prophylaxis of cardiovascular disorders and/or severe perioperative blood loss in man and animals, said compounds having a wide therapeutic range.

WO89/11852 describes, inter alia, substituted phenylalanine derivatives for treatment of pancreatitis, and WO 2007/07016 describes substituted thiophene derivatives as factor XIa inhibitors.

The invention provides compounds of the formula

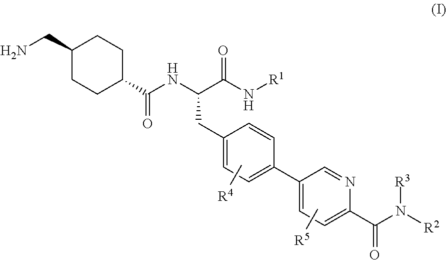

in which
R¹ is a group of the formula

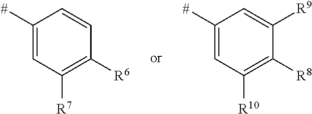

where # is the attachment site to the nitrogen atom,
R⁶ is 5-membered heteroaryl,
   where heteroaryl may be substituted by a substituent selected from the group consisting of oxo, chlorine, cyano, hydroxyl and C₁-C₃-alkyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl and methoxy, and in which alkyl is additionally substituted by 1 to 6 fluorine substituents,
R⁷ is hydrogen, fluorine or chlorine,
R⁸ and R⁹ together with the carbon atoms to which they are bonded form a 5-membered heterocycle,
   where the heterocycle may be substituted by 1 to 2 substituents selected independently from the group consisting of oxo, chlorine, cyano, hydroxyl, C₁-C₃-alkyl, pyrazolyl and pyridyl,
      in which alkyl may be substituted by 1 to 3 substituents selected independently from the group consisting of hydroxyl, amino, hydroxycarbonyl and methoxy,
      or
      in which alkyl may be substituted by 1 to 7 fluorine substituents,
      or
      in which alkyl is substituted by a substituent selected from the group consisting of hydroxyl, amino, hydroxycarbonyl and methoxy, and in which alkyl is additionally substituted by 1 to 6 fluorine substituents,
R₁₀ is hydrogen, fluorine or chlorine,
R² is hydrogen, C₁-C₆-alkyl, C₃-C₆-cycloalkyl, 4- to 9-membered heterocyclyl bonded via a carbon atom or 5- or 6-membered heteroaryl,
   where alkyl may be substituted by 1 to 2 substituents selected independently from the group consisting of fluorine, hydroxyl, amino, hydroxy carbonyl, C₁-C₃-alkylamino, difluoromethyl, trifluoromethyl, —(OCH₂CH₂)ₙ—OCH₃, —(OCH₂CH₂)ₘ—OH, trimethylaminium and pyrrolidinyl,
   in which n is a number from 1 to 6,
   in which m is a number from 1 to 6,
   and
   where cycloalkyl may be substituted by 1 to 2 substituents selected independently from the group consisting of oxo, fluorine, hydroxyl, amino, C₁-C₄-alkyl, C₁-C₃-alkylamino and morpholinyl,
      in which alkyl and alkylamino may be substituted by 1 to 5 fluorine substituents,
   and
   where heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
      where alkyl and alkylamino may be substituted by 1 to 5 substituents selected independently from the group consisting of hydroxyl and fluorine,
   and where heterocyclyl may additionally be substituted by 1 to 4 substituents selected independently from the group consisting of fluorine and methyl,
   and
   where heteroaryl may be substituted by 1 to 2 substituents selected independently from the group consisting of oxo, chlorine, cyano, hydroxyl and C₁-C₃-alkyl,
R³ is hydrogen or C₁-C₃-alkyl,
or
R² and R³ together with the nitrogen atom to which they are bonded form a 4- to 7-membered heterocycle,
   where the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R⁴ is hydrogen, fluorine, chlorine, methyl or methoxy,
R⁵ is hydrogen, fluorine, chlorine, C₁-C₄-alkyl, methoxy or trifluoromethyl,
and the salts thereof, the solvates thereof and the solvates of the salts thereof.

Inventive compounds are the compounds of the formula (I) and the salts, solvates and solvates of the salts thereof, and also the compounds encompassed by formula (I) and specified hereinafter as working example(s), and the salts, solvates and solvates of the salts thereof, to the extent that the compounds encompassed by formula (I) and specified hereinafter are not already salts, solvates and solvates of the salts.

The inventive compounds may, depending on their structure, exist in different stereoisomeric forms, i.e. in the form of configurational isomers or else optionally as conformational isomers (enantiomers and/or diastereomers, including those in the case of atropisomers). The present invention therefore encompasses the enantiomers and diastereomers, and the respective mixtures thereof. The stereoisomerically uniform constituents can be isolated from such mixtures of enantiomers and/or diastereomers in a known manner; chromatography processes are preferably used for this, especially HPLC chromatography on an achiral or chiral phase.

If the inventive compounds can occur in tautomeric forms, the present invention encompasses all the tautomeric forms.

The present invention also encompasses all suitable isotopic variants of the inventive compounds. An isotopic variant of an inventive compound is understood here as meaning a compound in which at least one atom within the inventive compound has been exchanged for another atom of the same atomic number, but with a different atomic mass than the atomic mass which usually or predominantly occurs in nature. Examples of isotopes which can be incorporated into an inventive compound are those of hydrogen, carbon, nitrogen, oxygen, phosphorus, sulphur, fluorine, chlorine, ¹³C, ¹⁴C, ¹⁵N, ¹⁷O, ¹⁸O, ³²P, ³³P, ³³S, ³⁴S, ³⁵S, bromine and iodine, such as ²H (deuterium), ³H (tritium), ³⁶S, ¹⁸F, ³⁶Cl, ⁸²Br, ¹²³I, ¹²⁴I, ¹²⁹I and ¹³¹I. Particular isotopic variants of an inventive compound, especially those in which one or more radioactive isotopes have been incorporated, may be beneficial, for example, for the examination of the mechanism of action or of the active ingredient distribution in the body; due to comparatively easy preparability and detectability, especially compounds labelled with ³H or ¹⁴C isotopes are suitable for this purpose. In addition, the incorporation of isotopes, for example of deuterium, can lead to particular therapeutic benefits as a consequence of greater metabolic stability of the compound, for example an extension of the half-life in the body or a reduction in the active dose required; such modifications of the inventive compounds may therefore in some cases also constitute a preferred embodiment of the present invention. Isotopic variants of the inventive compounds can be prepared by the processes known to those skilled in the art, for example by the methods described below and the procedures described in the working examples, by using corresponding isotopic modifications of the respective reagents and/or starting compounds.

In the context of the present invention, preferred salts are physiologically acceptable salts of the inventive compounds. The invention also encompasses salts which themselves are unsuitable for pharmaceutical applications but which can be used, for example, for the isolation or purification of the inventive compounds.

Physiologically acceptable salts of the inventive compounds include acid addition salts of mineral acids, carboxylic acids and sulphonic acids, for example salts of hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, trifluoroacetic acid, propionic acid, lactic acid, tartaric acid, malic acid, citric acid, fumaric acid, maleic acid and benzoic acid.

Physiologically acceptable salts of the inventive compounds also include salts of conventional bases, by way of example and with preference alkali metal salts (e.g. sodium and potassium salts), alkaline earth metal salts (e.g. calcium and magnesium salts) and ammonium salts derived from ammonia or organic amines having 1 to 16 carbon atoms, by way of example and with preference ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, arginine, lysine, ethylenediamine, N-methylpiperidine and choline.

In the context of the invention, solvates refer to those forms of the inventive compounds which, in the solid or liquid state, form a complex by coordination with solvent molecules. Hydrates are a specific form of the solvates in which the coordination is with water.

In addition, the present invention also encompasses prodrugs of the inventive compounds. The term “prodrugs” includes compounds which may themselves be biologically active or inactive but are converted to inventive compounds while resident in the body (for example metabolically or hydrolytically).

The two ways (A) and (B) of representing a 1,4-disubstituted cyclohexyl derivative shown below are equivalent to one another and identical, and in both cases describe a trans-1,4-disubstituted cyclohexyl derivative.

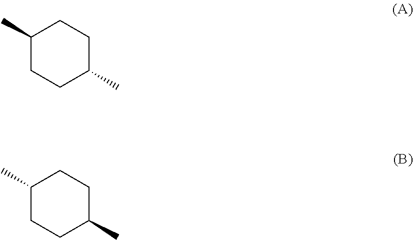

This applies especially to the structural element of tranexamamide, for example N-[(trans-4-{[(tert-butoxycarbonyl)amino]methyl}cyclohexyl)carbonyl and trans-4-(aminomethyl)-cyclohexyl]carbonyl}. In the present invention, representation (A) is used.

The three ways (C), (D) and (E) of representing tautomers of a triazole derivative shown below are equivalent to one another and identical and in all cases describe a 1,4-disubstituted triazole derivative.

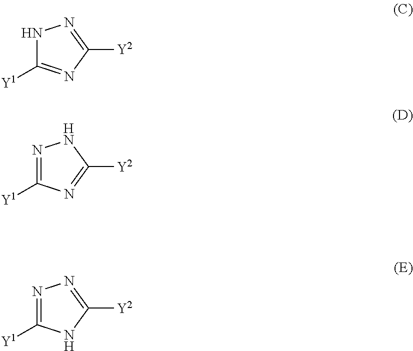

This applies especially to the following structural elements: 1H-1,2,4-triazol-3-yl, 1H-1,2,4-triazol-5-yl, 4H-1,2,4-triazol-3-yl and 4H-1,2,4-triazol-5-yl. Y¹ and Y² here are different substituents.

The two ways (F) and (G) of representing tautomers of a tetrazole derivative shown below are equivalent to one another and identical and in all cases describe a tetrazole derivative.

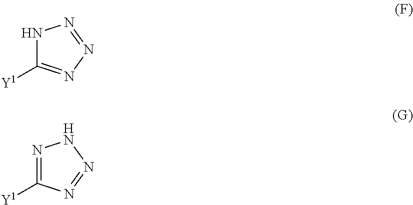

This applies especially to the following structural elements: 1H-tetrazol-5-yl and 2H-tetrazol-5-yl. Y³ here is the remainder of the compound.

The inventive compounds of the formula

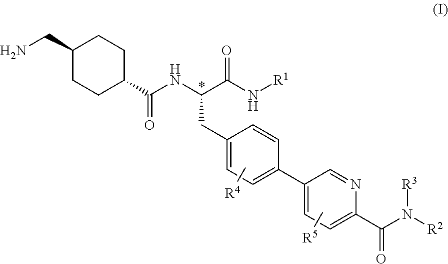

and all L-phenylalanine derivatives are described as the (S) configuration at the stereocentre marked with an * in the above formulae, since L-phenylalanine derivatives are introduced into the synthesis as central units. In the preparation of the inventive compounds, the coupling of the L-phenylalanine intermediates with the amine H₂N—R¹ can result in partial epimerization at the stereocentre marked by an *. Thus, a mixture of the inventive compounds of (S) enantiomer and (R) enantiomer can arise. The main component is the (S) enantiomer depicted in each case. The mixtures of (S) enantiomer and (R) enantiomer can be separated into their enantiomers by methods known to those skilled in the art, for example by chromatography on a chiral phase.

The enantiomers can be separated either directly after the coupling of the L-phenylalanine intermediates with the amine H₂N—R¹ or at a later synthesis intermediate, or else the inventive compounds can be separated themselves. Preference is given to the separation of the enantiomers directly after the coupling of the L-phenylalanine intermediates with the amine H₂N—R¹.

In the context of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

The terms “prevention”, “prophylaxis” or “preclusion” are used synonymously in the context of the present invention and refer to the avoidance or reduction of the risk of contracting, experiencing, suffering from or having a disease, a condition, a disorder, an injury or a health problem, or a development or advancement of such states and/or the symptoms of such states.

The treatment or prevention of a disease, a condition, a disorder, an injury or a health problem may be partial or complete.

In the context of the present invention, the substituents, unless specified otherwise, each have the following meaning:

Alkyl is a linear or branched alkyl radical having 1 to 6 carbon atoms, preferably 1 to 4 carbon atoms, more preferably 1 to 3 carbon atoms, by way of example and with preference methyl, ethyl, n-propyl, isopropyl, 2-methylprop-1-yl, n-butyl, tert-butyl, n-pentyl and n-hexyl.

Alkoxy is a linear or branched alkoxy radical having 1 to 6 carbon atoms, preferably 1 to 4 carbon atoms, more preferably 1 to 3 carbon atoms, by way of example and with preference methoxy, ethoxy, n-propoxy, isopropoxy, 2-methylprop-1-oxy, n-butoxy, tert-butoxy, n-pentoxy and n-hexoxy.

Alkylamino is an amino group having one or two independently selected, identical or different, linear or branched alkyl radicals each having 1 to 3 carbon atoms, for example and with preference methylamino, ethylamino, n-propylamino, isopropylamino, N,N-dimethylamino, N,N-diethylamino, N-ethyl-N-methylamino, N-methyl-N-n-propylamino, N-isopropyl-N-n-propylamino and N,N-diisopropylamino C₁-C₃-Alkylamino is, for example, a monoalkylamino radical having 1 to 3 carbon atoms or a dialkylamino radical having 1 to 3 carbon atoms in each alkyl radical.

Alkoxycarbonyl is a linear or branched alkoxy radical bonded by a carbonyl group, having 1 to 4 carbon atoms, preferably 1 to 3 carbon atoms, for example and with preference methoxycarbonyl, ethoxycarbonyl, n-propoxycarbonyl, isopropoxycarbonyl, n-butoxycarbonyl and tert-butoxycarbonyl.

Alkylaminocarbonyl is an amino group having one or two independently selected, identical or different, straight-chain or branched alkyl substituents each having 1 to 3 carbon atoms, bonded via a carbonyl group, for example and with preference methylaminocarbonyl, ethylaminocarbonyl, n-propylaminocarbonyl, isopropylaminocarbonyl, N,N-dimethylaminocarbonyl, N,N-diethylaminocarbonyl, N-ethyl-N-methylaminocarbonyl, N-methyl-N-n-propylaminocarbonyl, N-isopropyl-N-n-propylaminocarbonyl and N,N-diisopropylaminocarbonyl. C₁-C₃-Alkylaminocarbonyl is, for example, a monoalkylaminocarbonyl radical having 1 to 3 carbon atoms or a dialkylaminocarbonyl radical having 1 to 3 carbon atoms in each alkyl substituent.

Cycloalkyl is a monocyclic cycloalkyl group having 3 to 6 carbon atoms, preferred examples of cycloalkyl being cyclopropyl, cyclobutyl, cyclopentyl and cyclohexyl.

4- to 9-membered heterocyclyl bonded via a carbon atom in the definition of the R² radical is a saturated or partly unsaturated, monocyclic or bicyclic radical bonded via a carbon atom, having 4 to 9 ring atoms, preferably 5 or 6 ring atoms, and up to 3 heteroatoms and/or hetero groups, preferably 1 or 2 heteroatoms and/or hetero groups, from the group of S, O, N, SO and SO₂, where one nitrogen atom may also form an N-oxide, for example and with preference azetidinyl, pyrrolidinyl, piperidinyl, tetrahydropyranyl, 3-azabicyclo[3.1.0]hex-6-yl, 8-azabicyclo[3.2.1]oct-3-yl, 3-oxa-9-azabicyclo[3.3.1]non-7-yl and azepanyl, more preferably pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl.

5- or 6-membered heteroaryl in the definition of the R² radical is an aromatic monocyclic radical having 5 or 6 ring atoms and up to 4 heteroatoms and/or hetero groups from the group of S, O, N, SO and SO₂, where one nitrogen atom may also form an N-oxide, for example and with preference thienyl, furyl, pyrrolyl, thiazolyl, oxazolyl, isoxazolyl, oxadiazolyl, pyrazolyl, imidazolyl, triazolyl, tetrazolyl, pyridyl and pyridazinyl, more preferably pyrazolyl.

4- to 7-membered heterocycle in the definition of the R² and R³ radicals is a saturated or partly unsaturated, monocyclic or bicyclic radical having 4 to 7 ring atoms, preferably 5 or 6 ring atoms, and up to 3 heteroatoms and/or hetero groups, preferably 1 or 2 heteroatoms and/or hetero groups, from the group of S, O, N, SO and SO₂, where one nitrogen atom may also form an N-oxide, for example and with preference azetidinyl, pyrrolidinyl, morpholinyl, thiomorpholinyl, piperidinyl, piperazinyl, 3-azabicyclo[3.1.0]hex-6-yl, 8-azabicyclo[3.2.1]oct-3-yl and azepanyl, more preferably piperazinyl.

5-membered heteroaryl in the definition of the R⁶ radical is an aromatic monocyclic radical having 5 ring atoms and up to 4 heteroatoms and/or hetero groups from the group of S, O, N, SO and SO₂, where one nitrogen atom may also form an N-oxide, for example and with preference thienyl, furyl, pyrrolyl, thiazolyl, oxazolyl, isoxazolyl, oxadiazolyl, pyrazolyl, imidazolyl, triazolyl and tetrazolyl, more preferably triazolyl and tetrazolyl, most preferably tetrazolyl.

5-membered heterocycle in the definition of the R⁸ and R⁹ radicals is a saturated, partly unsaturated or aromatic monocyclic radical having 5 ring atoms and up to 2 heteroatoms and/or hetero groups from the group of S, O, N, SO and SO₂, where one nitrogen atom may also form an N-oxide. This 5-membered heterocycle together with the phenyl ring to which it is bonded is, for example and with preference, 2,3-dihydro-1-benzothiophen-5-yl, 1,3-dihydro-2-benzothiophen-5-yl, 2,3-dihydro-1-benzofuran-5-yl, 1,3-dihydro-2-benzofuran-5-yl, indolin-5-yl, isoindolin-5-yl, 2,3-dihydro-1H-indazol-5-yl, 2,3-dihydro-1H-benzimidazol-5-yl, 1,3-dihydro-2, 1-benzoxazol-5-yl, 2,3-dihydro-1,3-benzoxazol-5-yl, 1,3-dihydro-2,1-benzothiazol-5-yl, 2,3-dihydro-1,3-benzothiazol-5-yl, 1H-benzimidazol-5-yl, 1H-indazol-5-yl, 1,2-benzoxazol-5-yl, indol-5-yl, isoindol-5-yl, benzofuran-5-yl, benzothiophen-5-yl, 2,3-dihydro-1-benzothiophen-6-yl, 1,3-dihydro-2-benzothiophen-6-yl, 2,3-dihydro-1-benzofuran-6-yl, 1,3-dihydro-2-benzofuran-6-yl, indolin-6-yl, isoindolin-6-yl, 2,3-dihydro-1H-indazol-6-yl, 2,3-dihydro-1H-benzimidazol-6-yl, 1,3-dihydro-2, 1-benzoxazol-6-yl, 2,3-dihydro-1,3-benzoxazol-6-yl, 1,3-dihydro-2,1-benzothiazol-6-yl, 2,3-dihydro-1,3-benzothiazol-6-yl, 1H-benzimidazol-6-yl, 1H-indazol-6-yl, 1,2-benzoxazol-6-yl, indol-6-yl, isoindol-6-yl, benzofuran-6-yl and benzothiophen-6-yl, more preferably 2,3-dihydro-1H-benzimidazol-5-yl, 2,3-dihydro-1H-indazol-6-yl and 1H-benzimidazol-6-yl, most preferably 2,3-dihydro-1H-benzimidazol-5-yl and 2,3-dihydro-1H-indazol-6-yl.

In the formulae of the group which may represent R¹, the end point of the line marked by # does not represent a carbon atom or a CH₂ group, but is part of the bond to the atom to which R¹ is bonded.

Preference is given to compounds of the formula (I) in which
R¹ is a group of the formula

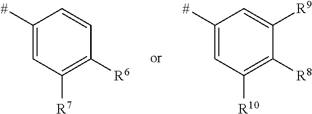

where # is the attachment site to the nitrogen atom,
R⁶ is 5-membered heteroaryl,
   where heteroaryl may be substituted by a substituent selected from the group consisting of oxo, chlorine and C₁-C₃-alkyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R⁷ is hydrogen or fluorine,
R⁸ and R⁹ together with the carbon atoms to which they are bonded form a 5-membered heterocycle,
   where the heterocycle may be substituted by 1 to 2 substituents selected independently from the group consisting of oxo, chlorine, hydroxyl, C₁-C₃-alkyl, pyrazolyl and pyridyl,
      in which alkyl may be substituted by 1 to 2 substituents selected independently from the group consisting of hydroxycarbonyl and methoxy,
      or
      in which alkyl may be substituted by 1 to 7 fluorine substituents,
      or
      in which alkyl is substituted by a hydroxycarbonyl substituent and in which alkyl is additionally substituted by 1 to 6 fluorine substituents,
R¹⁰ is hydrogen or fluorine,
R² is hydrogen, C₁-C₆-alkyl, C₃-C₆-cycloalkyl, 4- to 9-membered heterocyclyl bonded via a carbon atom or 5- or 6-membered heteroaryl,
   where alkyl may be substituted by 1 to 2 substituents selected independently from the group consisting of fluorine, hydroxyl, amino, hydroxycarbonyl, C₁-C₃-alkylamino, difluoromethyl, trifluoromethyl, —(OCH₂CH₂)ₘ—OCH₃ and pyrrolidinyl,
   in which n is a number from 1 to 6,
   and
   where cycloalkyl may be substituted by 1 to 2 substituents selected independently from the group consisting of oxo, fluorine, hydroxyl, amino, C₁-C₄-alkyl, C₁-C₃-alkylamino and morpholinyl,
      in which alkyl and alkylamino may be substituted by 1 to 5 fluorine substituents,
   and
   where heterocyclyl may be substituted by 1 to 2 substituents selected independently from the group consisting of oxo, fluorine and C₁-C₄-alkyl,
   and
   where heteroaryl may be substituted by 1 to 2 substituents selected independently from the group consisting of C₁-C₃-alkyl,
R³ is hydrogen, methyl or ethyl,
or
R² and R³ together with the nitrogen atom to which they are bonded form a 4- to 7-membered heterocycle,
   where the heterocycle may be substituted by 1 to 2 substituents selected independently from the group consisting of oxo and C₁-C₄-alkyl,
R⁴ is hydrogen, fluorine, chlorine, methyl or methoxy,
R⁵ is hydrogen, fluorine, chlorine, C₁-C₄-alkyl, methoxy or trifluoromethyl,
and the salts thereof, the solvates thereof and the solvates of the salts thereof.

Preference is also given to compounds of the formula (I) in which
R¹ is a group of the formula

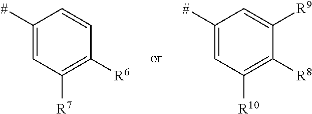

where # is the attachment site to the nitrogen atom,
R⁶ is 5-membered heteroaryl,
R⁷ is hydrogen,
R⁸ and R⁹ together with the carbon atoms to which they are bonded form a 5-membered heterocycle,
   where the heterocycle may be substituted by an oxo substituent,
R¹⁰ is hydrogen,
R² is C₁-C₆-alkyl, cyclohexyl, 4- to 9-membered heterocyclyl bonded via a carbon atom or 5- or 6-membered heteroaryl,
   where alkyl may be substituted by a substituent selected from the group consisting of hydroxyl and C₁-C₃-alkylamino,
   and
   where cyclohexyl may be substituted by a substituent selected from the group consisting of hydroxyl, amino, C₁-C₃-alkylamino and morpholinyl,
   and
   where heterocyclyl may be substituted by 1 to 2 substituents independently selected from the group consisting of oxo, fluorine and methyl,
   and
   where heteroaryl may be substituted by 1 to 2 methyl substituents,
R³ is hydrogen,
or
R² and R³ together with the nitrogen atom to which they are bonded form a 4- to 7-membered heterocycle,
   where the heterocycle may be substituted by 1 to 2 substituents selected independently from the group consisting of oxo and methyl,
R⁴ is hydrogen,
R⁵ is methyl or trifluoromethyl,
and the salts thereof, the solvates thereof and the solvates of the salts thereof.

Preference is also given to compounds of the formula (I) in which
R¹ is a group of the formula

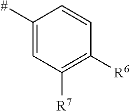

where # is the attachment site to the nitrogen atom,
R⁶ is tetrazolyl,
R⁷ is hydrogen,
or
R¹ is 2,3-dihydro-1H-benzimidazol-5-yl or 2,3-dihydro-1H-indazol-6-yl,
   where 2,3-dihydro-1H-benzimidazol-5-yl and 2,3-dihydro-1H-indazol-6-yl may be substituted by an oxo substituent,
R² is C₁-C₆-alkyl, cyclohexyl, heterocyclyl bonded via a carbon atom and selected from the group consisting of pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl, or pyrazolyl,
   where alkyl may be substituted by a substituent selected from the group consisting of hydroxyl and C₁-C₃-alkylamino,
   and
   where cyclohexyl may be substituted by a substituent selected from the group consisting of hydroxyl, amino, C₁-C₃-alkylamino and morpholinyl,
   and
   where pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl may be substituted by 1 to 2 substituents selected independently from the group consisting of oxo, fluorine and methyl,
   and
   where pyrazolyl may be substituted by 1 to 2 methyl substituents,
R³ is hydrogen,
or
R² and R³ together with the nitrogen atom to which they are bonded form a piperazinyl,
   where piperazinyl may be substituted by 1 to 2 substituents selected independently from the group consisting of oxo and methyl,
R⁴ is hydrogen,
R⁵ is methyl or trifluoromethyl,
and the salts thereof, the solvates thereof and the solvates of the salts thereof.

Preference is also given to compounds of the formula (I) in which
R¹ is a group of the formula

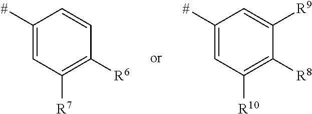

where # is the attachment site to the nitrogen atom,
R⁶ is 5-membered heteroaryl,
R⁷ is hydrogen,
R⁸ and R⁹ together with the carbon atoms to which they are bonded form a 5-membered heterocycle,
   where the heterocycle may be substituted by an oxo substituent,
R¹⁰ is hydrogen,
R² is C₁-C₆-alkyl, cyclopropyl, cyclobutyl, cyclohexyl, 4- to 9-membered heterocyclyl bonded via a carbon atom or 5- or 6-membered heteroaryl,
   where alkyl may be substituted by a substituent selected from the group consisting of hydroxyl, C₁-C₃-alkylamino and trifluoromethyl,
   and
   where cyclohexyl may be substituted by a substituent selected from the group consisting of hydroxyl, amino, C₁-C₃-alkylamino and morpholinyl,
   and
   where heterocyclyl may be substituted by 1 to 2 substituents independently selected from the group consisting of oxo, fluorine and methyl,
   and
   where heteroaryl may be substituted by 1 to 2 methyl substituents,
R³ is hydrogen,
or
R² and R³ together with the nitrogen atom to which they are bonded form a 4- to 7-membered heterocycle,
   where the heterocycle may be substituted by 1 to 2 substituents selected independently from the group consisting of oxo and methyl,
R⁴ is hydrogen,
R⁵ is methyl or trifluoromethyl,
and the salts thereof, the solvates thereof and the solvates of the salts thereof.

Preference is also given to compounds of the formula (I) in which
R¹ is a group of the formula

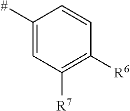

where # is the attachment site to the nitrogen atom,
R⁶ is tetrazolyl,
R⁷ is hydrogen,
or
R¹ is 2,3-dihydro-1H-benzimidazol-5-yl or 2,3-dihydro-1H-indazol-6-yl,
   where 2,3-dihydro-1H-benzimidazol-5-yl and 2,3-dihydro-1H-indazol-6-yl may be substituted by an oxo substituent,
R² is C₁-C₆-alkyl, cyclopropyl, cyclobutyl, cyclohexyl, heterocyclyl bonded via a carbon atom and selected from the group consisting of pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl, or pyrazolyl,
   where alkyl may be substituted by a substituent selected from the group consisting of hydroxyl, C₁-C₃-alkylamino and trifluoromethyl,
   and
   where cyclohexyl may be substituted by a substituent selected from the group consisting of hydroxyl, amino, C₁-C₃-alkylamino and morpholinyl,
   and
   where pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl may be substituted by 1 to 2 substituents selected independently from the group consisting of oxo, fluorine and methyl,
   and
   where pyrazolyl may be substituted by 1 to 2 methyl substituents,
R³ is hydrogen,
or
R² and R³ together with the nitrogen atom to which they are bonded form a piperazinyl,
   where piperazinyl may be substituted by 1 to 2 substituents selected independently from the group consisting of oxo and methyl,
R⁴ is hydrogen,
R⁵ is methyl or trifluoromethyl,
and the salts thereof, the solvates thereof and the solvates of the salts thereof.

Preference is given to compounds of the formula (I) in which
R¹ is 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl or 1H-indazol-6-yl,
   where the 5-membered heterocycle in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by an oxo substituent,
   and
   where the benzyl ring in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by a chlorine substituent,
R² is ethyl, isopropyl, cyclopropyl, cyclobutyl, cyclohexyl, or heterocyclyl bonded via a carbon atom, selected from the group of pyrrolidinyl and piperidinyl,
   where ethyl is substituted by a trifluoromethyl substituent,
   and
   where cyclohexyl is substituted by a substituent selected from the group consisting of hydroxyl, amino and C₁-C₃-alkylamino,
   and
   where pyrrolidinyl and piperidinyl may be substituted by 1 to 2 substituents independently selected from the group consisting of oxo, fluorine and C₁-C₄-alkyl,
R³ is hydrogen,
R⁴ is hydrogen or fluorine,
R⁵ is methyl,
and the salts thereof, the solvates thereof and the solvates of the salts thereof.

Preference is given to compounds of the formula (I) in which
R¹ is 2,3-dihydro-1H-benzimidazol-5-yl, 2,3-dihydro-1H-indazol-6-yl or 1H-indazol-6-yl,
   where the 5-membered heterocycle in 2,3-dihydro-1H-benzimidazol-5-yl, 2,3-dihydro-1H-indazol-6-yl and 1H-indazol-6-yl may be substituted by an oxo substituent,
   and
   where the benzyl ring in 2,3-dihydro-1H-benzimidazol-5-yl may be substituted by a chlorine substituent,
R² is ethyl, isopropyl, cyclopropyl or cyclobutyl,
   where ethyl is substituted by a trifluoromethyl substituent,
R³ is hydrogen,
R⁴ is hydrogen or fluorine,
R⁵ is methyl,
and the salts thereof, the solvates thereof and the solvates of the salts thereof.

Preference is also given to compounds of the formula (I) in which
R¹ is a group of the formula

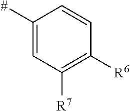

where # is the attachment site to the nitrogen atom,
R⁶ is tetrazolyl,
and
R′ is hydrogen.

Preference is also given to compounds of the formula (I) in which
R¹ is 2,3-dihydro-1H-benzimidazol-5-yl or 2,3-dihydro-1H-indazol-6-yl,
   where 2,3-dihydro-1H-benzimidazol-5-yl and 2,3-dihydro-1H-indazol-6-yl may be substituted by an oxo substituent.

Preference is also given to compounds of the formula (I) in which
R¹ is 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl or 1H-indazol-6-yl,
   where the 5-membered heterocycle in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by an oxo substituent,
   and
   where the benzyl ring in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by a chlorine substituent.

Preference is also given to compounds of the formula (I) in which
R¹ is 2,3-dihydro-1H-benzimidazol-5-yl, 2,3-dihydro-1H-indazol-6-yl or 1H-indazol-6-yl,
   where the 5-membered heterocycle in 2,3-dihydro-1H-benzimidazol-5-yl, 2,3-dihydro-1H-indazol-6-yl and 1H-indazol-6-yl may be substituted by an oxo substituent,
   and
   where the benzyl ring in 2,3-dihydro-1H-benzimidazol-5-yl may be substituted by a chlorine substituent.

Preference is also given to compounds of the formula (I) in which
R² is C₁-C₆-alkyl, cyclohexyl, heterocyclyl bonded via a carbon atom and selected from the group consisting of pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl, or pyrazolyl,
   where alkyl may be substituted by a substituent selected from the group consisting of hydroxyl and C₁-C₃-alkylamino,
   and
   where cyclohexyl may be substituted by a substituent selected from the group consisting of hydroxyl, amino, C₁-C₃-alkylamino and morpholinyl,
   and
   where pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl may be substituted by 1 to 2 substituents selected independently from the group consisting of oxo, fluorine and methyl,
   and
   where pyrazolyl may be substituted by 1 to 2 methyl substituents.

Preference is also given to compounds of the formula (I) in which
R² is ethyl, isopropyl, cyclopropyl, cyclobutyl, cyclohexyl, or heterocyclyl bonded via a carbon atom, selected from the group of pyrrolidinyl and piperidinyl,
   where ethyl is substituted by a trifluoromethyl substituent,
   and
   where cyclohexyl is substituted by a substituent selected from the group consisting of hydroxyl, amino and C₁-C₃-alkylamino,
   and
   where pyrrolidinyl and piperidinyl may be substituted by 1 to 2 substituents independently selected from the group consisting of oxo, fluorine and C₁-C₄-alkyl.

Preference is also given to compounds of the formula (I) in which
R² is ethyl, isopropyl, cyclopropyl or cyclobutyl,
   where ethyl is substituted by a trifluoromethyl substituent.

Preference is also given to compounds of the formula (I) in which R³ is hydrogen.

Preference is also given to compounds of the formula (I) in which
R² and R³ together with the nitrogen atom to which they are bonded form a piperazinyl,
   where piperazinyl may be substituted by 1 to 2 substituents selected independently from the group consisting of oxo and methyl.

Preference is also given to compounds of the formula (I) in which R⁴ is hydrogen.

Preference is also given to compounds of the formula (I) in which R⁵ is methyl or trifluoromethyl.

Preference is also given to compounds of the formula (I) in which R⁵ is methyl.

The individual radical definitions specified in the particular combinations or preferred combinations of radicals are, independently of the particular combinations of the radicals specified, also replaced as desired by radical definitions of other combinations.

Very particular preference is given to combinations of two or more of the abovementioned preferred ranges.

The invention further provides a process for preparing the compounds of the formula (I), or the salts thereof, solvates thereof and the solvates of the salts thereof, wherein the compounds of the formula

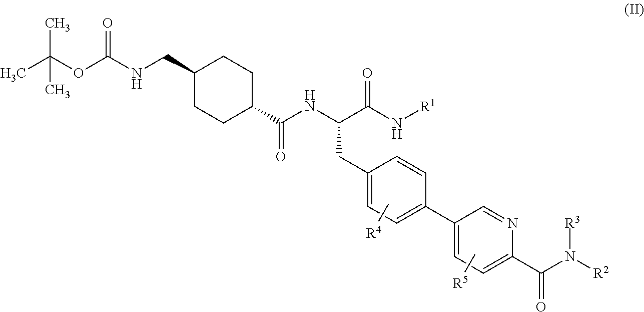

in which
R¹, R², R³, R⁴ and R⁵ are each as defined above,
are reacted with an acid.

The reaction is generally effected in inert solvents, preferably within a temperature range from room temperature to 60° C. at standard pressure.

Inert solvents are, for example, halogenated hydrocarbons such as dichloromethane, trichloromethane, tetrachloromethane or 1,2-dichloroethane, or ethers such as tetrahydrofuran or dioxane, preference being given to dioxane.

Acids are, for example, trifluoroacetic acid or hydrogen chloride in dioxane, preference being given to hydrogen chloride in dioxane.

The compounds of the formula (II) are known or can be prepared by reacting

[A] compounds of the formula

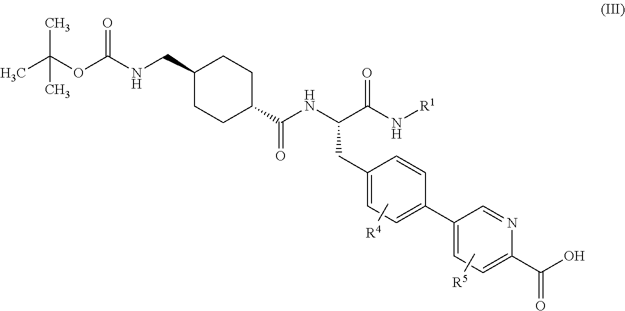

in which
R¹, R⁴ and R⁵ have the meaning given above,
with compounds of the formula

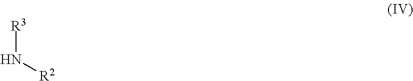

in which
R² and R³ have the meaning given above,
in the presence of a dehydrating reagent,
or
[B] compounds of the formula

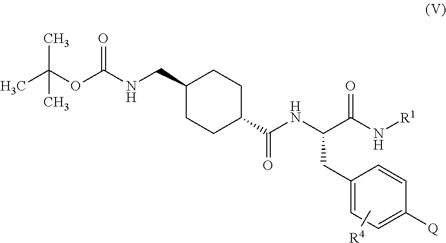

in which
R¹ and R⁴ are each as defined above, and
Q¹ is —B(OH)₂, a boronic ester, preferably pinacol boronate, or —BF₃⁻K⁺,
with compounds of the formula

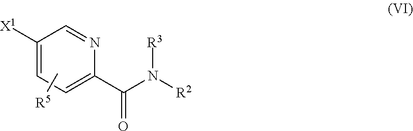

in which
R², R³ and R⁵ have the meaning given above and
X¹ is bromine or iodine,
under Suzuki coupling conditions,
or
[C] compounds of the formula

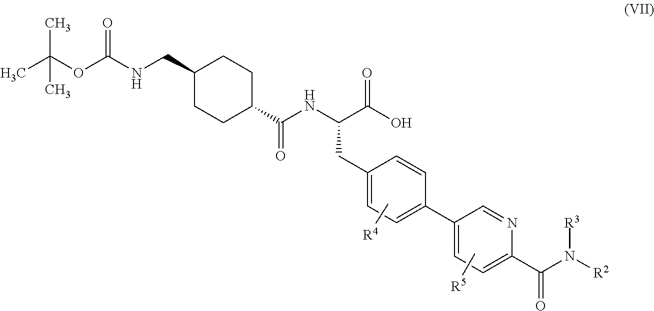

in which
R², R³, R⁴ and R⁵ are each as defined above,
with compounds of the formula

H₂N—R¹ (VIII)

in which
IV is as defined above
in the presence of a dehydrating reagent.

The reaction in process [A] is generally effected in inert solvents, optionally in the presence of a base, preferably within a temperature range from 0° C. to the reflux of the solvents at standard pressure.

Suitable dehydrating reagents here are, for example, carbodiimides, for example N,N′-diethyl-, N,N′-dipropyl-, N,N′-diisopropyl- and N,N′-dicyclohexylcarbodiimide, N-(3-dimethylaminoisopropyl)-N′-ethylcarbodiimide hydrochloride (EDC) (optionally in the presence of pentafluorophenol (PFP)), N-cyclohexylcarbodiimid-N′-propyloxymethyl-polystyrene (PS-carbodiimide) or carbonyl compounds such as carbonyldiimidazole, or 1,2-oxazolium compounds such as 2-ethyl-5-phenyl-1,2-oxazolium 3-sulphate or 2-tert-butyl-5-methyl-isoxazolium perchlorate, or acylamino compounds such as 2-ethoxy-1-ethoxycarbonyl-1,2-dihydroquinoline, or propanephosphonic anhydride, or isobutyl chloroformate, or bis-(2-oxo-3-oxazolidinyl)phosphoryl chloride or benzotriazolyloxytri(dimethylamino)phosphonium hexafluorophosphate, or O-(benzotriazol-1-yl)-N,N,N′,N′-tetramethyluronium hexafluorophosphate (HBTU), 2-(2-oxo-1-(2H)-pyridyl)-1, 1,3,3-tetramethyluronium tetrafluoroborate (TPTU), (benzotriazol-1-yloxy)bisdimethylaminomethylium fluoroborate (TBTU) or O-(7-azabenzotriazol-1-yl)-N,N,N′,N′-tetramethyluronium hexafluorophosphate (HATU), or 1-hydroxybenzotriazole (HOBt), or benzotriazol-1-yloxytris(dimethylamino)phosphonium hexafluorophosphate (BOP), or ethyl cyano(hydroxyimino)acetate (Oxyma), or (1-cyano-2-ethoxy-2-oxoethylideneaminooxy) dimethylaminomorpholinocarbenium hexafluorophosphate (COMU), or N-[(dimethylamino)(3H-[1,2,3]triazolo[4,5-b]pyridin-3-yloxy)methylidene]-N-methylmethanaminium hexafluorophosphate, or 2,4,6-tripropyl-1,3,5,2,4,6-trioxatriphosphinane 2,4,6-trioxide (T3P), or mixtures of these, preference being given to N-[(dimethylamino)(3H-[1,2,3]triazolo[4,5-b]pyridin-3-yloxy)methylidene]-N-methylmethanaminium hexafluorophosphate or 2,4,6-tripropyl-1,3,5,2,4,6-trioxatriphosphinane 2,4,6-trioxide (T3P).

Bases are, for example, alkali metal carbonates, for example sodium carbonate or potassium carbonate, or sodium hydrogencarbonate or potassium hydrogencarbonate, or organic bases such as trialkylamines, for example triethylamine, N-methylmorpholine, N-methylpiperidine, 4-dimethylaminopyridine or diisopropylethylamine, preference being given to diisopropylethylamine.

Inert solvents are, for example, halogenated hydrocarbons such as dichloromethane or trichloromethane, hydrocarbons such as benzene, or other solvents such as nitromethane, tetrahydrofuran, dioxane, dimethylformamide, dimethyl sulphoxide, acetonitrile or pyridine, or mixtures of the solvents, preference being given to tetrahydrofuran or dimethylformamide or a mixture of dimethylformamide and pyridine.

The compounds of the formula (IV) are known, can be synthesized from the corresponding starting compounds by known processes or can be prepared analogously to the processes described in the Examples section.

The reaction in process [B] is generally effected in inert solvents, in the presence of a catalyst, optionally in the presence of an additional reagent, optionally in a microwave, preferably within a temperature range from room temperature to 150° C. at standard pressure to 3 bar.

Catalysts are, for example, palladium catalysts customary for Suzuki reaction conditions, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphineferrocenyl)palladium(II) chloride, 1,3-bis(2,6-diisopropylphenyl) imidazol-2-ylidene (1,4-napththoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1, 1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)], preference being given to tetrakistriphenylphosphinepalladium(0), [1,1-bis-(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)].

Additional reagents are, for example, potassium acetate, caesium carbonate, potassium carbonate or sodium carbonate, potassium tert-butoxide, caesium fluoride or potassium phosphate, which may be present in aqueous solution; preferred additional reagents are those such as potassium acetate or a mixture of potassium acetate and sodium carbonate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, or mixtures of the solvents with alcohols such as methanol or ethanol and/or water, preference being given to toluene, dimethylformamide or dimethyl sulphoxide.

The compounds of the formula (VI) are known, can be synthesized from the corresponding starting compounds by known processes or can be prepared analogously to the processes described in the Examples section.

The reaction in process [C] is effected as described for process [A].

The compounds of the formula (VIII) are known, can be synthesized from the corresponding starting compounds by known processes or can be prepared analogously to the processes described in the Examples section.

The compounds of the formula (III) are known or can be prepared by reacting

[D] compounds of the formula

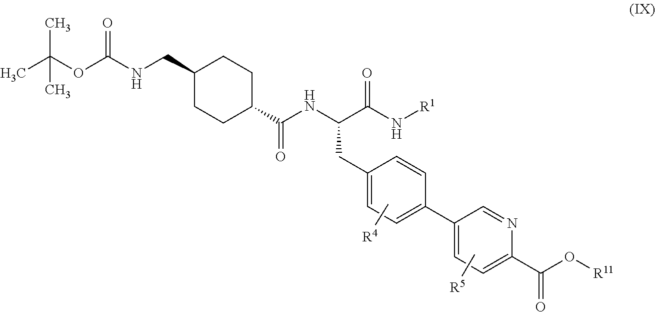

in which
R′, R⁴ and R⁵ have the meaning given above and
R¹¹ is methyl or ethyl,
with a base,
or
[E] reacting compounds of the formula

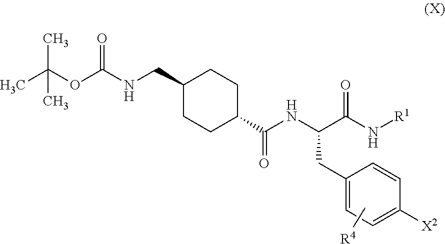

in which
R¹ and R⁴ are each as defined above, and
X² is bromine or iodine,
with compounds of the formula

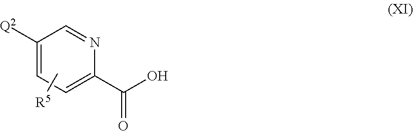

in which
R⁵ is as defined above, and
Q² is B(OH)₂, a boronic ester, preferably pinacol boronate, or —BF₃⁻K⁺, under Suzuki coupling conditions.

The reaction in process [D] is generally effected in inert solvents, preferably within a temperature range from room temperature up to the reflux of the solvents at standard pressure.

Inert solvents are, for example, halogenated hydrocarbons such as dichloromethane, trichloromethane, tetrachloromethane or 1,2-dichloroethane, alcohols such as methanol or ethanol, ethers such as diethyl ether, methyl tert-butyl ether, 1,2-dimethoxyethane, dioxane or tetrahydrofuran, or other solvents such as dimethylformamide, dimethylacetamide, acetonitrile or pyridine, or mixtures of solvents, or mixtures of solvent with water, preference being given to a mixture of tetrahydrofuran and water.

Bases are, for example, alkali metal hydroxides such as sodium hydroxide, lithium hydroxide or potassium hydroxide, or alkali metal carbonates such as caesium carbonate, sodium carbonate or potassium carbonate, or alkoxides such as potassium tert-butoxide or sodium tert-butoxide, preference being given to sodium hydroxide and lithium hydroxide.

The reaction in process [E] is effected as described for process [B].

The compounds of the formula (XI) are known, can be synthesized from the corresponding starting compounds by known processes or can be prepared analogously to the processes described in the Examples section.

The compounds of the formula (IX) are known or can be prepared by reacting

[F] compounds of the formula (X) with compounds of the formula

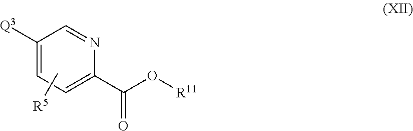

in which
R⁵ is as defined above,
R¹¹ is methyl or ethyl, and
Q³ is B(OH)₂, a boronic ester, preferably pinacol boronate, or —BF₃⁻K⁺, under Suzuki coupling conditions,
or
[G] reacting compounds of the formula

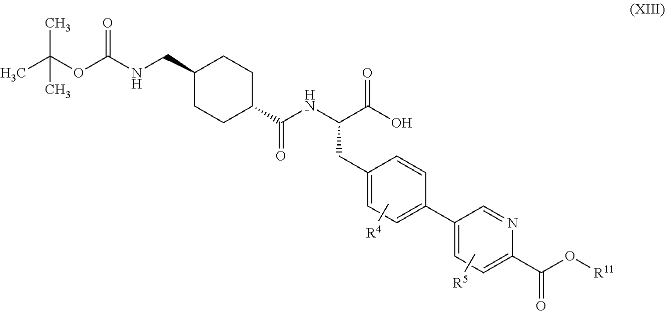

in which
R⁴ and R⁵ are each as defined above, and
R¹¹ is methyl or ethyl,
with compounds of the formula (VIII) in the presence of a dehydrating reagent.

The reaction in process [F] is effected as described for process [B].

The compounds of the formula (XII) are known, can be synthesized from the corresponding starting compounds by known processes or can be prepared analogously to the processes described in the Examples section.

The reaction in process [G] is effected as described for process [A].

The compounds of the formula (X) are known or can be prepared by reacting compounds of the formula

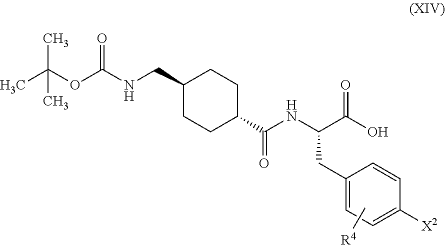

in which
R⁴ is as defined above, and
X² is bromine or iodine,
with compounds of the formula (VIII) in the presence of a dehydrating reagent.

The reaction is effected as described for process [A].

The compounds of the formula (XIV) are known, can be synthesized from the corresponding starting compounds by known processes or can be prepared analogously to the processes described in the Examples section.

The compounds of the formula (XIII) are known or can be prepared by reacting compounds of the formula (XIV) with compounds of the formula (XII) under Suzuki coupling conditions.

The reaction is effected as described for process [B].

The compounds of the formula (V) are known or can be prepared by reacting compounds of the formula (X) with 4,4,4′,4′,5,5,5′,5′-octamethyl-2,2′-bi-1,3,2-dioxaborolane.

The reaction is generally effected in inert solvents, in the presence of a catalyst, optionally in the presence of an additional reagent, optionally in a microwave, preferably within a temperature range from room temperature to 150° C. at standard pressure to 3 bar. Hydrolysis in an acidic medium affords the corresponding boronic acids. Workup with potassium dihydrogenfluoride solution (KHF₂ solution) affords the corresponding trifluoroborates.

Catalysts are, for example, palladium catalysts customary for the borylation of aryl halides, preference being given to catalysts such as dichlorobis(triphenylphosphine)palladium, tetrakistriphenylphosphinepalladium(0), palladium(II) acetate/triscyclohexylphosphine, tris(dibenzylideneacetone)dipalladium, bis(diphenylphosphineferrocenyl)palladium(II) chloride, 1,3-bis(2,6-diisopropylphenyl) imidazol-2-ylidene (1,4-napththoquinone)palladium dimer, allyl(chloro)(1,3-dimesityl-1,3-dihydro-2H-imidazol-2-ylidene)palladium, palladium(II) acetate/dicyclohexyl(2′,4′,6′-triisopropyl-biphenyl-2-yl)phosphine, [1,1-bis(diphenylphosphino)ferrocene]palladium(II) chloride monodichloromethane adduct or XPhos precatalyst [(2′-aminobiphenyl-2-yl)(chloro)palladium dicyclohexyl(2′,4′,6′-triisopropylbiphenyl-2-yl)phosphine (1:1)], preference being given to tetrakistriphenylphosphinepalladium(0) and [1,1-bis-(diphenylphosphino)ferrocene]palladium(II) chloride.

Additional reagents are, for example, potassium acetate, caesium carbonate, potassium carbonate or sodium carbonate, potassium tert-butoxide or sodium tert-butoxide, caesium fluoride, potassium phosphate or potassium phenoxide, preference being given to potassium acetate.

Inert solvents are, for example, ethers such as dioxane, tetrahydrofuran or 1,2-dimethoxyethane, hydrocarbons such as benzene, xylene or toluene, or carboxamides such as dimethylformamide or dimethylacetamide, alkyl sulphoxides such as dimethyl sulphoxide, or N-methylpyrrolidone or acetonitrile, preference being given to dioxane, dimethylformamide or dimethyl sulphoxide.
Literature: K. L. Billingslay, T. E. Barde, S. L Buchwald, Angew. Chem. 2007, 119, 5455 or T. Graening, Nachrichten aus der Chemie, January 2009, 57, 34.

The compounds of the formula (VII) are known or can be prepared by reacting compounds of the formula (XIV) with compounds of the formula

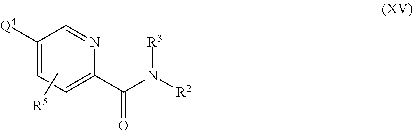

in which
R², R³ and R⁵ have the meaning given above and
Q⁴ is —B(OH)₂, a boronic ester, preferably pinacol boronate, or —BF₃⁻K⁺, under Suzuki coupling conditions.

The reaction is effected as described for process [B].

The compounds of the formula (XV) are known, can be synthesized from the corresponding starting compounds by known processes or can be prepared analogously to the processes described in the Examples section.

The preparation of the starting compounds and of the compounds of the formula (I) can be illustrated by the synthesis scheme below.

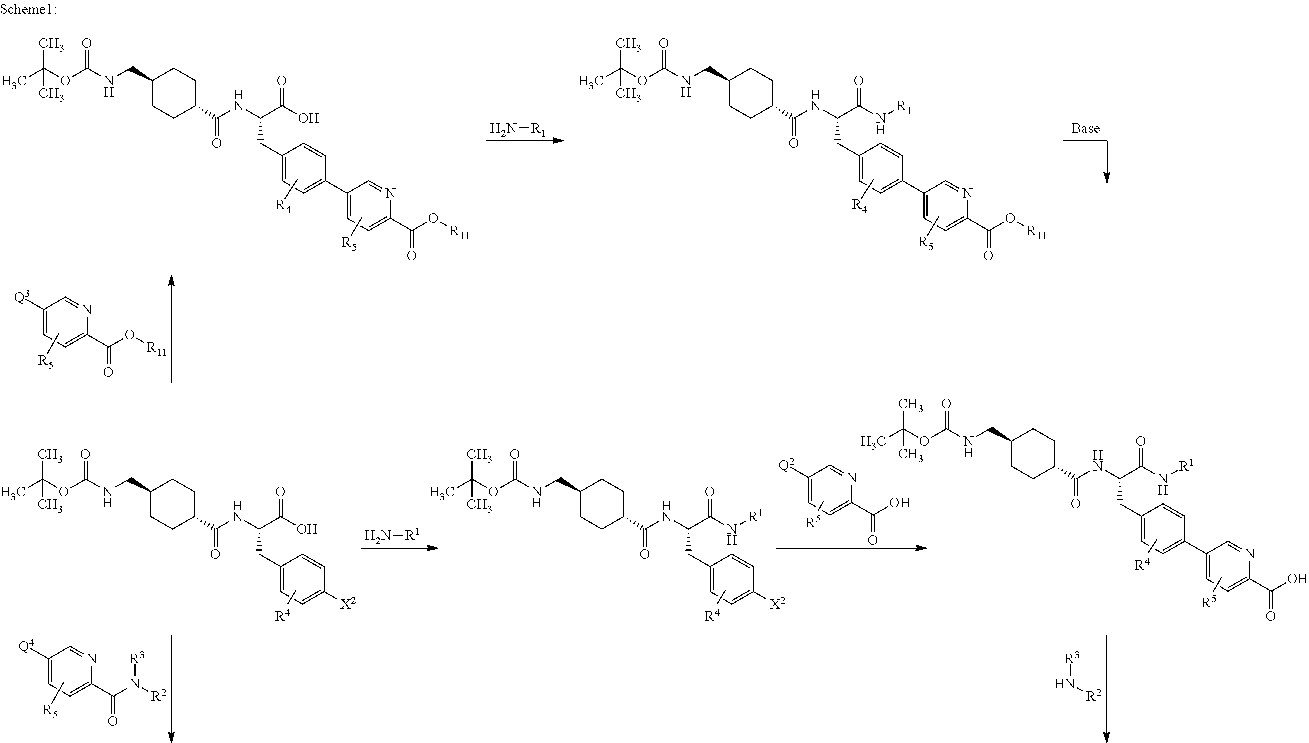

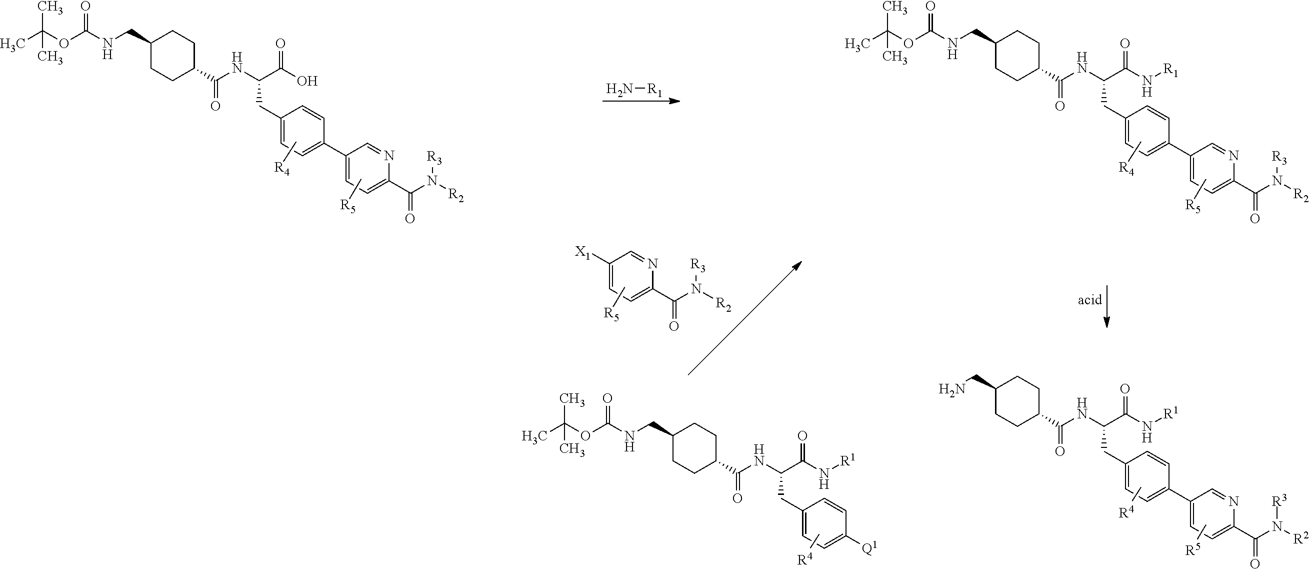

The inventive compounds have an unforeseeable useful spectrum of pharmacological activity and good pharmacokinetic properties. They are compounds that influence the proteolytic activity of the serine proteases FXIa and kallikrein, and possibly plasmin. The inventive compounds inhibit the enzymatic cleavage of substrates that assume a major role in the activation of the blood coagulation cascade and platelet aggregation. If the inventive compounds inhibit plasmin activity, the result is inhibition of fibrinolysis.

They are therefore suitable for use as medicaments for treatment and/or prophylaxis of diseases in man and animals.

The present invention further provides for the use of the inventive compounds for treatment and/or prophylaxis of disorders, especially cardiovascular disorders, preferably thrombotic or thromboembolic disorders and/or thrombotic or thromboembolic complications.

“Thromboembolic disorders” in the sense of the present invention include in particular disorders such as acute coronary syndrome (ACS), ST-segment elevation myocardial infarction (STEMI) and non-ST-segment elevation myocardial infarction (non-STEMI), stable angina pectoris, unstable angina pectoris, reocclusions and restenoses after coronary interventions such as angioplasty, stent implantation or aortocoronary bypass, peripheral arterial occlusion diseases, pulmonary embolisms, venous thromboses, especially in deep leg veins and renal veins, transitory ischaemic attacks and also thrombotic and thromboembolic stroke.

The inventive compounds are therefore also suitable for the prevention and treatment of cardiogenic thromboembolisms, for example brain ischaemias, stroke and systemic thromboembolisms and ischaemias, in patients with acute, intermittent or persistent cardial arrhythmias, for example atrial fibrillation, and those undergoing cardioversion, and also in patients with heart valve disorders or with artificial heart valves.

In addition, the inventive compounds are suitable for the treatment and prevention of disseminated intravascular coagulation (DIC) which may occur in connection with sepsis inter alfa, but also owing to surgical interventions, neoplastic disorders, burns or other injuries and may lead to severe organ damage through microthrombosis.

Thromboembolic complications are also encountered in microangiopathic haemolytic anaemias, extracorporeal circulatory systems, such as haemodialysis, and also prosthetic heart valves.

In addition, the inventive compounds are also used for influencing wound healing, for the prophylaxis and/or treatment of atherosclerotic vascular disorders and inflammatory disorders, such as rheumatic disorders of the locomotive system, coronary heart diseases, of heart failure, of hypertension, of inflammatory disorders, for example asthma, inflammatory pulmonary disorders, glomerulonephritis and inflammatory intestinal disorders, for example Crohn's disease or ulcerative colitis or acute renal failure, and additionally likewise for the prophylaxis and/or treatment of dementia disorders, for example Alzheimer's disease. In addition, the inventive compounds can be used for inhibiting tumour growth and the formation of metastases, for microangiopathies, age-related macular degeneration, diabetic retinopathy, diabetic nephropathy and other microvascular disorders, and also for the prevention and treatment of thromboembolic complications, for example venous thromboembolisms, for tumour patients, especially those undergoing major surgery or chemo- or radiotherapy.

In addition, the inventive compounds are also suitable for the prophylaxis and/or treatment of pulmonary hypertension.

The term “pulmonary hypertension” includes certain forms of pulmonary hypertension, as determined, for example, by the World Health Organization (WHO). Examples include pulmonary arterial hypertension, pulmonary hypertension associated with disorders of the left heart, pulmonary hypertension associated with pulmonary disorders and/or hypoxia and pulmonary hypertension owing to chronic thromboembolisms (CTEPH).

“Pulmonary arterial hypertension” includes idiopathic pulmonary arterial hypertension (IPAH, formerly also referred to as primary pulmonary hypertension), familial pulmonary arterial hypertension (FPAH) and associated pulmonary-arterial hypertension (APAH), which is associated with collagenoses, congenital systemic-pulmonary shunt vitia, portal hypertension, HIV infections, the ingestion of certain drugs and medicaments, with other disorders (thyroid disorders, glycogen storage disorders, Morbus Gaucher, hereditary teleangiectasia, haemoglobinopathies, myeloproliferative disorders, splenectomy), with disorders having a significant venous/capillary contribution, such as pulmonary-venoocclusive disorder and pulmonary-capillary haemangiomatosis, and also persisting pulmonary hypertension of neonatants.

Pulmonary hypertension associated with disorders of the left heart includes a diseased left atrium or ventricle and mitral or aorta valve defects.

Pulmonary hypertension associated with pulmonary disorders and/or hypoxia includes chronic obstructive pulmonary disorders, interstitial pulmonary disorder, sleep apnoea syndrome, alveolar hypoventilation, chronic high-altitude sickness and inherent defects.

Pulmonary hypertension owing to chronic thromboembolisms (CTEPH) comprises the thromboembolic occlusion of proximal pulmonary arteries, the thromboembolic occlusion of distal pulmonary arteries and non-thrombotic pulmonary embolisms (tumour, parasites, foreign bodies).

The present invention further provides for the use of the inventive compounds for production of medicaments for treatment and/or prophylaxis of pulmonary hypertension associated with sarcoidosis, histiocytosis X and lymphangiomatosis.

In addition, the inventive substances may also be useful for treatment of pulmonary and hepatic fibroses.

In addition, the inventive compounds may also be suitable for treatment and/or prophylaxis of disseminated intravascular coagulation in the context of an infectious disease, and/or of systemic inflammatory syndrome (SIRS), septic organ dysfunction, septic organ failure and multiorgan failure, acute respiratory distress syndrome (ARDS), acute lung injury (ALI), septic shock and/or septic organ failure.

In the course of an infection, there may be a generalized activation of the coagulation system (disseminated intravascular coagulation or consumption coagulopathy, hereinbelow referred to as “DIC”) with microthrombosis in various organs and secondary haemorrhagic complications. Moreover, there may be endothelial damage with increased permeability of the vessels and seeping of fluids and proteins into the extravasal lumen. As the infection progresses, there may be failure of an organ (for example kidney failure, liver failure, respiratory failure, central-nervous deficits and cardiovascular failure) or multiorgan failure.

In the case of DIC, there is a massive activation of the coagulation system at the surface of damaged endothelial cells, the surfaces of foreign bodies or injured extravascular tissue. As a consequence, there is coagulation in small vessels of various organs with hypoxia and subsequent organ dysfunction. This can be prevented by the inventive compounds. A secondary effect is the consumption of coagulation factors (for example factor X, prothrombin and fibrinogen) and platelets, which reduces the coagulability of the blood and may result in heavy bleeding.

In addition, the inventive compounds are also useful for the prophylaxis and/or treatment of hyperfibrinolysis. The prophylaxis and/or treatment may reduce or eliminate severe perioperative blood loss. Severe bleeding occurs in major operations, for example coronary artery bypass surgery, transplants or hysterectomy, and in the event of trauma, in the event of haemorrhagic shock or in the event of postpartum haemorrhage. In the aforementioned indications, there may be perioperative use of extracorporeal circulation systems or filter systems, for example heart and lung machines, haemofiltration, haemodialysis, extracorporeal membrane oxygenation or a ventricular support system, for example artificial heart. This additionally requires anticoagulation, for which the inventive compounds can also be used.

The inventive compounds are also suitable for anticoagulation during kidney replacement procedures, for example in the case of continuous veno-venous haemofiltration or intermittent haemodialysis.

The inventive compounds can additionally also be used for preventing coagulation ex vivo, for example for preserving blood and plasma products, for cleaning/pretreating catheters and other medical auxiliaries and instruments, for coating synthetic surfaces of medical auxiliaries and instruments used in vivo or ex vivo or for biological samples which could contain factor XIa.

The present invention further provides for the use of the inventive compounds for treatment and/or prophylaxis of disorders, especially the disorders mentioned above.

The present invention further provides for the use of the inventive compounds for production of a medicament for treatment and/or prophylaxis of disorders, especially of the aforementioned disorders.

The present invention further provides a method for treatment and/or prophylaxis of disorders, especially the disorders mentioned above, using a therapeutically effective amount of an inventive compound.

The present invention further provides the inventive compounds for use in a method for treatment and/or prophylaxis of disorders, especially of the aforementioned disorders, using a therapeutically effective amount of an inventive compound.

The present invention further provides medicaments comprising an inventive compound and one or more further active ingredients.

The present invention further provides a method for preventing the coagulation of blood in vitro, especially in banked blood or biological samples which could contain factor XIa, which is characterized in that an anticoagulatory amount of the inventive compound is added.

The present invention further provides medicaments comprising an inventive compound and one or more further active ingredients, especially for treatment and/or prophylaxis of the disorders mentioned above. Preferred examples of active ingredients suitable for combinations include:
lipid-lowering substances, especially HMG-CoA (3-hydroxy-3-methylglutaryl-coenzyme A) reductase inhibitors, for example lovastatin (Mevacor), simvastatin (Zocor), pravastatin (Pravachol), fluvastatin (Lescol) and atorvastatin (Lipitor);
coronary therapeutics/vasodilatators, especially ACE (angiotensin converting enzyme) inhibitors, for example captopril, lisinopril, enalapril, ramipril, cilazapril, benazepril, fosinopril, quinapril and perindopril, or AII (angiotensin II) receptor antagonists, for example embusartan, losartan, valsartan, irbesartan, candesartan, eprosartan and temisartan, or (3-adrenoceptor antagonists, for example carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, for example prazosine, bunazosine, doxazosine and terazosine, or diuretics, for example hydrochlorothiazide, furosemide, bumetanide, piretanide, torasemide, amiloride and dihydralazine, or calcium channel blockers, for example verapamil and diltiazem, or dihydropyridine derivatives, for example nifedipin (Adalat) and nitrendipine (Bayotensin), or nitro preparations, for example isosorbide 5-mononitrate, isosorbide dinitrate and glycerol trinitrate, or substances causing an increase in cyclic guanosine monophosphate (cGMP), for example stimulators of soluble guanylate cyclase, for example riociguat;

plasminogen activators (thrombolytics/fibrinolytics) and compounds which promote thrombolysis/fibrinolysis such as inhibitors of the plasminogen activator inhibitor (PAI inhibitors) or inhibitors of the thrombin-activated fibrinolysis inhibitor (TAFI inhibitors), for example tissue plasminogen activator (t-PA), streptokinase, reteplase and urokinase;

anticoagulatory substances (anticoagulants), for example heparin (UFH), low-molecular-weight heparins (NMH), for example tinzaparin, certoparin, parnaparin, nadroparin, ardeparin, enoxaparin, reviparin, dalteparin, danaparoid, semuloparin (AVE 5026), adomiparin (M118) and EP-42675/ORG42675;

direct thrombin inhibitors (DTI), for example Pradaxa (dabigatran), atecegatran (AZD-0837), DP-4088, SSR-182289A, argatroban, bivalirudin and tanogitran (BIBT-986 and prodrug BIBT-1011), hirudin;

direct factor Xa inhibitors for example, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux;

platelet aggregation-inhibiting substances (platelet aggregation inhibitors, thrombocyte aggregation inhibitors), for example acetylsalicylic acid (for example Aspirin), ticlopidine (Ticlid), clopidogrel (Plavix), prasugrel, ticagrelor, cangrelor, elinogrel, vorapaxar;

fibrinogen receptor antagonists (glycoprotein-IIb/IIIa antagonists), for example abciximab, eptifibatide, tirofiban, lamifiban, lefradafiban and fradafiban;

and also antiarrhythmics;

various antibiotics or antifungal medicaments, either as calculated therapy (prior to the presence of the microbial diagnosis) or as specific therapy;

vasopressors, for example norepinephrine, dopamine and vasopressin;

inotropic therapy, for example dobutamine;

corticosteroids, for example hydrocortisone and fludrocortisone;

recombinant human activated protein C, for example Xigris;

blood products, for example erythrocyte concentrates, thrombocyte concentrates, erythropietin and fresh frozen plasma.

“Combinations” for the purpose of the invention mean not only dosage forms which contain all the components (so-called fixed combinations) and combination packs which contain the components separate from one another, but also components which are administered simultaneously or sequentially, provided that they are used for prophylaxis and/or treatment of the same disease. It is likewise possible to combine two or more active ingredients with one another, meaning that they are thus each in two-component or multicomponent combinations.

The inventive compounds may act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The inventive compounds can be administered in suitable administration forms for these administration routes.

Suitable administration forms for oral administration are those which function according to the prior art and deliver the inventive compounds rapidly and/or in modified fashion, and which contain the inventive compounds in crystalline and/or amorphized and/or dissolved form, for example tablets (uncoated or coated tablets, for example having enteric coatings or coatings which are insoluble or dissolve with a delay and control the release of the inventive compound), tablets which disintegrate rapidly in the mouth, or films/wafers, films/lyophilizates, capsules (for example hard or soft gelatin capsules), sugar-coated tablets, granules, pellets, powders, emulsions, suspensions, aerosols or solutions.

Parenteral administration can be accomplished with avoidance of an absorption step (for example by an intravenous, intraarterial, intracardiac, intraspinal or intralumbar route) or with inclusion of an absorption (for example by an intramuscular, subcutaneous, intracutaneous, percutaneous or intraperitoneal route). Suitable administration forms for parenteral administration include injection and infusion formulations in the form of solutions, suspensions, emulsions, lyophilizates or sterile powders.

Parenteral administration is preferred.

For the other administration routes, suitable examples are inhalation medicaments (including powder inhalers, nebulizers), nasal drops, solutions or sprays; tablets for lingual, sublingual or buccal administration, films/wafers or capsules, suppositories, ear or eye preparations, vaginal capsules, aqueous suspensions (lotions, shaking mixtures), lipophilic suspensions, ointments, creams, transdermal therapeutic systems (for example patches), milk, pastes, foams, dusting powders, implants or stents.

The inventive compounds can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colourants (e.g. inorganic pigments, for example iron oxides) and flavour and/or odour correctants.

The present invention further provides medicaments comprising at least one inventive compound, preferably together with one or more inert nontoxic pharmaceutically suitable excipients, and the use thereof for the purposes mentioned above.

In the case of parenteral administration, it has generally been found to be advantageous to administer amounts of about 5 to 250 mg every 24 hours to achieve effective results. In the case of oral administration, the amount is about 5 to 500 mg every 24 hours.

In spite of this, it may be necessary to deviate from the amounts specified, specifically depending on body weight, administration route, individual behaviour towards the active ingredient, type of formulation, and time or interval of administration.

Unless stated otherwise, the percentages in the tests and examples which follow are percentages by weight; parts are parts by weight. Solvent ratios, dilution ratios and concentration data for the liquid/liquid solutions are in each case based on volume. “w/v” means “weight/volume”. For example, “10% w/v” means: 100 ml of solution or suspension comprise 10 g of substance.

### A) EXAMPLES

### Abbreviations

bs/br. s. broad singlet (in NMR)
bd broad doublet (in NMR)
cat. catalytic
CI chemical ionization (in MS)
dd doublet of doublets (in NMR)
DMF dimethylformamide
DMSO dimethyl sulphoxide
dt doublet of triplets (in NMR)
of th. of theory (in yield)
EI electron impact ionization (in MS)
eq. equivalent(s)
ESI electrospray ionization (in MS)
h hour(s)
HATU O-(7-azabenzotriazol-1-yl)-N,N,N′,N′-tetramethyluronium hexafluorophosphate
HPLC high-pressure high-performance liquid chromatography
LC-MS liquid chromatography-coupled mass spectroscopy
m multiplet (in NMR)
M molar
min minute(s)
MS mass spectrometry
N normal
NMR nuclear magnetic resonance spectrometry
q quartet (in NMR)
quant. quantitative
quint quintet (in NMR)
RT room temperature
Rₜ retention time (in HPLC)
s singlet (in NMR)
TFA trifluoroacetic acid
THF tetrahydrofuran
UV ultraviolet spectrometry

### HPLC and LC/MS Methods:

Method 1 (LC-MS): Instrument: Waters ACQUITY SQD UPLC System; column: Waters Acquity UPLC HSS T3 1.8μ 50×1 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow rate: 0.40 ml/min; UV detection: 210-400 nm.
Method 2 (LC-MS): Instrument: Micromass Quattro Premier with Waters UPLC Acquity; column: Thermo Hypersil GOLD 1.9μ 50 mm×1 mm; eluent A: 1 l water+0.5 ml 50% formic acid, eluent B: 1 l acetonitrile+0.5 ml 50% formic acid; gradient: 0.0 min 97% A→0.5 min 97% A→3.2 min 5% A→4.0 min 5% A; oven: 50° C.; flow rate: 0.3 ml/min; UV detection: 210 nm.
Method 3 (LC-MS): Instrument: Waters ACQUITY SQD UPLC System; column: Waters Acquity UPLC HSS T3 1.8μ 30×2 mm; eluent A: 1 l water+0.25 ml 99% formic acid, eluent B: 1 l acetonitrile+0.25 ml 99% formic acid; gradient: 0.0 min 90% A→1.2 min 5% A→2.0 min 5% A; oven: 50° C.; flow rate: 0.60 ml/min; UV detection: 208-400 nm.
Method 4 (LC-MS): Instrument: Waters Acquity UPLC-MS SQD 3001; column: Acquity UPLC BEH C18 1.7μ 50 mm×2.1 mm; eluent A: water+0.1% formic acid, eluent B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.
Method 5 (LC-MS): Instrument: Waters Acquity UPLC-MS SQD 3001; column: Acquity UPLC BEH C18 1.7μ 50 mm×2.1 mm; eluent A: water+0.2% ammonia, eluent B: acetonitrile; gradient: 0-1.6 min 1-99% B, 1.6-2.0 min 99% B; flow rate: 0.8 ml/min; temperature: 60° C.; injection: 2 μl; DAD scan: 210-400 nm; ELSD.
Method 6 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile, gradient: A 95%/B 5%→A 55%/B 45%; flow rate: 150 ml/min; UV detection: 254 nm.
Method 7 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile; gradient: A 90%/B 10%→A 50%/B 50%; flow rate: 150 ml/min; UV detection: 254 nm.
Method 8 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile; gradient: A 85%/B 15%→A 45%/B 55%; flow rate: 150 ml/min; UV detection: 254 nm.
Method 9 (HPLC): System: Labomatic HD-3000 HPLC gradient pump, Labomatic Labocol Vario-2000 fraction collector; column: Chromatorex C-18 125 mm×30 mm, eluent A: 0.1% formic acid in water, eluent B: acetonitrile; gradient: A 80%/B 20%→A 40%/B 60%; flow rate: 150 ml/min; UV detection: 254 nm.
Method 10 (HPLC): Instrument: Waters SQD autopurification system; column: Waters XBridge C18 5μ 100 mm×30 mm; eluent A: water+0.1% formic acid (99%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm.
Method 11 (HPLC): Instrument: Waters SQD autopurification system; column: Waters XBridge C18 5μ 100 mm×30 mm; eluent A: water+0.2% ammonia (32%), eluent B: acetonitrile; gradient: 0-8.0 min 1-100% B, 8.0-10.0 min 100% B; flow rate 50.0 ml/min; temperature: RT; injection: 2500 μl; DAD scan: 210-400 nm.
Method 12 (LC-MS): MS instrument: Waters (Micromass) QM; HPLC instrument: Agilent 1100 series; column: Agilent ZORBAX Extend-C18 3.0 mm×50 mm 3.5 micron; eluent A: 1 l water+0.01 mol ammonium carbonate, eluent B: 1 l acetonitrile; gradient: 0.0 min 98% A→0.2 min 98% A→3.0 min 5% A→4.5 min 5% A; oven: 40° C.; flow rate: 1.75 ml/min; UV detection: 210 nm
Method 13 (LC-MS): Instrument: Waters ACQUITY SQD UPLC System; column: Waters Acquity UPLC HSS T3 1.8μ 50 mm×1 mm; eluent A: 1 l water+0.25 ml 99% formic acid, eluent B: 1 l acetonitrile+0.25 ml 99% formic acid; gradient: 0.0 min 95% A→6.0 min 5% A→7.5 min 5% A; oven: 50° C.; flow rate: 0.35 ml/min; UV detection: 210-400 nm.
Method 14 (LC-MS): MS instrument: Waters (Micromass) Quattro Micro; HPLC instrument: Agilent 1100 Series; column: YMC-Triart C18 3μ 50 mm×3 mm; eluent A: 1 l water+0.01 mol ammonium carbonate, eluent B: 1 l acetonitrile; gradient: 0.0 min 0% A→2.75 min 5% A→4.5 min 5% A; oven: 40° C.; flow rate: 1.25 ml/min; UV detection: 210 nm
Method 15 (HPLC): System: Labomatic HD-3000 HPLC gradient pump, Labomatic Labocol Vario-2000 fraction collector; column: Chromatorex C-18 125 mm×30 mm; eluent A: 0.1% formic acid in water, eluent B: acetonitrile; gradient: A 60%/B 40%→A 20%/B 80%; flow rate: 150 ml/min; UV detection: 254 nm.

Microwave: The microwave reactor used was an instrument of the Biotage™ Initiator type.

When inventive compounds are purified by preparative HPLC by the above-described methods in which the eluents contain additives, for example trifluoroacetic acid, formic acid or ammonia, the inventive compounds may be obtained in salt form, for example as trifluoroacetate, formate or ammonium salt, if the inventive compounds contain a sufficiently basic or acidic functionality. Such a salt can be converted to the corresponding free base or acid by various methods known to the person skilled in the art. Weaker salts can be converted to the corresponding chlorides by addition of a little hydrochloride.

If, in the synthesis intermediates and working examples of the invention described below, a compound is given in the form of a salt of the corresponding base or acid, the exact stoichiometric composition of such a salt as obtained by the respective preparation and/or purification process is generally not known. Unless specified in more detail, additions to names and structural formulae, such as “hydrochloride”, “trifluoroacetate”, “sodium salt” or “x HCl”, “x CF₃COOH”, “x Na⁺” are not to be understood stoichiometrically in the case of such salts, but have only descriptive character with regard to the salt-forming components comprised therein.

This applies correspondingly if the synthesis intermediates and working examples or salts thereof were obtained by the preparation and/or purification processes described in the form of solvates, for example hydrates, whose stoichiometric composition (if of a defined type) is not known.

If the starting compounds and examples contain an L-phenylalanine derivative as the central unit, the corresponding stereocentre is described as the (S) configuration. In the absence of further information, there was no check in individual cases as to whether partial epimerization of the stereocentre took place in the coupling of the L-phenylalanine intermediates with the amine H₂N—R′. Thus, a mixture of the inventive compounds of (S) enantiomer and (R) enantiomer may be present. The main component is the (S) enantiomer depicted in each case.

### Starting Compounds

### Example 1A

### Methyl 4-bromo-N-[(trans-4-{[(tert-butoxycarbonyl)amino]methyl}cyclohexyl)carbonyl]-L-phenylalaninate

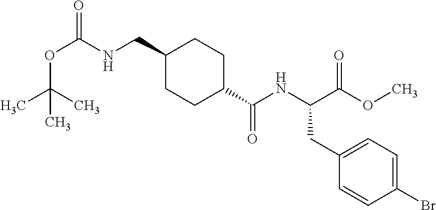

A solution of methyl 4-bromo-L-phenylalaninate (250 g, 874 mmol) and trans-4-{[(tert-butoxycarbonyl)amino]methyl}cyclohexanecarboxylic acid (225 g, 874 mmol) in ethyl acetate (5012 ml) was admixed with N,N-diisopropylethylamine (381 ml, 2186 mmol). The suspension was admixed dropwise with a 2,4,6-tripropyl-1,3,5,2,4,6-trioxatriphosphinane 2,4,6-trioxide solution (50% in dimethylformamide, 766 ml, 1312 mmol) and then the mixture was stirred at RT for 3 h. The reaction mixture was then stirred into water and extracted three times with ethyl acetate. The organic phase was washed with saturated aqueous sodium hydrogencarbonate solution, saturated aqueous ammonium chloride solution, and saturated aqueous sodium chloride solution. The solution was dried over sodium sulphate and the solvent was removed. This gave 420 g (97% of theory) of the title compound.

¹H NMR (400 MHz, DMSO-d₆): δ=ppm 0.68-0.92 (m, 2H), 1.04-1.32 (m, 4H), 1.37 (s, 9H), 1.48-1.73 (m, 4H), 2.03 (m, 1H), 2.74 (m, 2H), 2.78-2.90 (m, 1H), 2.94-3.05 (m, 1H), 4.36-4.50 (m, 1H), 6.72-6.85 (m, 1H), 7.17 (d, 2H), 7.46 (d, 2H), 8.15 (d, 1H)

LC-MS (Method 1): Rₜ=1.14 min; MS (ESIpos): m/z=497 [M+H]⁺.

### Example 2A

### 4-Bromo-N-[(trans-4-{[(tert-butoxycarbonyl)amino]methyl}cyclohexyl)carbonyl]-L-phenylalanine

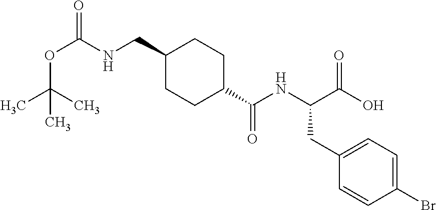

A solution of methyl 4-bromo-N-[(trans-4-{[(tert-butoxycarbonyl)amino]methyl}-cyclohexyl)carbonyl]-L-phenylalaninate in tetrahydrofuran (3000 ml) was admixed with a solution of lithium hydroxide (72 g, 3015 mmol) in water (600 ml). The suspension was stirred at RT for 16 h. The reaction mixture was acidified with 1N hydrochloric acid solution and admixed with ethyl acetate. The organic phase was washed with saturated aqueous sodium chloride solution and dried over sodium sulphate, and the solvent was removed. This gave 284 g (97% of theory) of the title compound.

¹H NMR (400 MHz, DMSO-d₆): δ=ppm 0.71-0.90 (m, 2H), 1.22 (d, 4H), 1.37 (s, 9H), 1.45-1.73 (m, 5H), 2.03 (m, 1H), 2.67-2.88 (m, 3H), 2.95-3.09 (m, 1H), 4.38 (m, 1H), 6.77 (s, 1H), 7.17 (d, 2H), 7.46 (d, 2H), 7.99 (d, 1H), 12.65 (br. s, 1H)

LC-MS (Method 1): Rₜ=1.03 min; MS (ESIneg): m/z=481 [M−H]⁻.

### Example 3A

### 4-Bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide

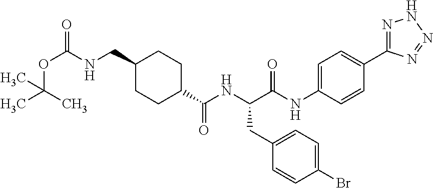

A solution of 4-bromo-N-[(trans-4-{[(tert-butoxycarbonyl)amino]methyl}-cyclohexyl)-carbonyl]-L-phenylalanine (11 g, 22 mmol) and 4-(1H-tetrazol-5-yl)aniline (4 g, 24 mmol) in dimethylformamide (161 ml) was admixed with N,N-diisopropylethylamine (9.6 ml, 55 mmol). The suspension was admixed dropwise at 0° C. with a 2,4,6-tripropyl-1,3,5,2,4,6-trioxatriphosphinane 2,4,6-trioxide solution (50% in dimethylformamide, 16.9 g, 27 mmol) and then the mixture was stirred at RT for 16 h. The reaction mixture was stirred into ethyl acetate (13000 ml) and extracted three times with water (1570 ml each time). The organic phase was dried with sodium sulphate and the solvent was removed. The crude product was stirred with acetonitrile and filtered off with suction. This gave 11.4 g (78% of theory) of the title compound.

¹H NMR (400 MHz, DMSO-d₆): δ=ppm 0.67-0.90 (m, 2H), 1.24 (m, 4H), 1.37 (s, 9H), 1.51-1.74 (m, 4H), 2.02-2.17 (m, 1H), 2.71-2.79 (m, 2H), 2.79-2.89 (m, 1H), 2.99-3.06 (m, 1H), 3.06-3.16 (m, 1H), 3.51-3.67 (m, 1H), 4.55-4.74 (m, 1H), 6.01-6.02 (m, 1H), 6.69-6.84 (m, 1H), 7.21-7.32 (m, 2H), 7.43-7.55 (m, 2H), 7.64-7.76 (m, 2H), 7.88-7.99 (m, 2H), 8.03-8.14 (m, 1H), 10.25 (s, 1H)

LC-MS (Method 1): Rₜ=1.07 min; MS (ESIneg): m/z=624 [M−H]⁻.

### Example 4A

### 4-Bromo-N-alpha-[(trans-4-{[(tert-butoxycarbonyl)amino]methyl}cyclohexyl)carbonyl]-N-(3-oxo-2,3-dihydro-1H-indazol-6-yl)-L-phenylalaninamide

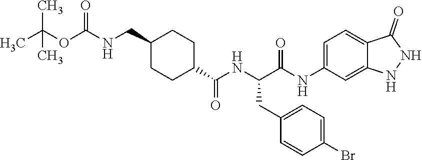

A solution of 4-bromo-N-[(trans-4-{[(tert-butoxycarbonyl)amino]methyl}-cyclohexyl)-carbonyl]-L-phenylalanine (1500 mg, 3 mmol) and 6-amino-1,2-dihydro-3H-indazol-3-one (555 mg, 24 mmol) in ethyl acetate (21 ml) was admixed with N,N-diisopropylethylamine (1.4 ml, 7.8 mmol). The suspension was admixed with a 2,4,6-tripropyl-1,3,5,2,4,6-trioxatriphosphinane 2,4,6-trioxide solution (50% in dimethylformamide, 2.2 ml, 3.7 mmol) and with dimethylformamide until dissolution, and then the mixture was stirred at RT for 16 h. The reaction mixture was stirred into ethyl acetate, and washed twice with water and once with saturated aqueous sodium chloride solution. The organic phase was dried with sodium sulphate and the solvent was removed. The crude product was stirred with acetonitrile and filtered off with suction. The residue was separated twice by means of preparative HPLC (eluent: acetonitrile/water with 0.1% TFA (gradient)). The crude product was stirred with methanol and filtered off with suction. This gave 202 mg (11% of theory) of the title compound.

¹H NMR (400 MHz, DMSO-d₆): δ=ppm 0.69-0.89 (m, 2H), 1.04-1.29 (m, 3H), 1.37 (s, 9H), 1.67 (m, 4H), 2.04-2.17 (m, 1H), 2.75 (m, 3H), 2.94-3.07 (m, 1H), 4.54-4.75 (m, 1H), 6.68-6.83 (m, 1H), 6.96 (dd, 1H), 7.25 (d, 2H), 7.39-7.56 (m, 3H), 7.84 (s, 1H), 8.09 (d, 1H), 10.20 (s, 1H), 11.08 (br. s, 1H)

LC-MS (Method 1): Rₜ=1.00 min; MS (ESIpos): m/z=614 [M+H]⁺.

### Example 5A

### 4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide

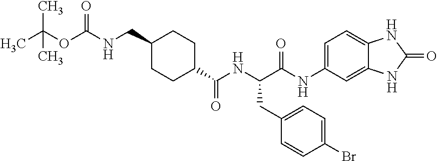

A solution of 4-bromo-N-[(trans-4-{[(tert-butoxycarbonyl)amino]methyl}-cyclohexyl)-carbonyl]-L-phenylalanine (5000 mg, 10 mmol) and 5-amino-1,3-dihydro-2H-benzimidazol-2-one (1851 mg, 12 mmol) in ethyl acetate (70 ml) was admixed with N,N-diisopropylethylamine (4.5 ml, 26 mmol). The suspension was admixed with a 2,4,6-tripropyl-1,3,5,2,4,6-trioxatriphosphinane 2,4,6-trioxide solution (50% in dimethylformamide, 7898 mg, 12 mmol) and with dimethylformamide (about 20 ml) until dissolution, and then the mixture was stirred at RT for 16 h. The reaction mixture was stirred into ethyl acetate (600 ml), and washed three times with water (300 ml) and once with saturated aqueous sodium chloride solution (250 ml). The precipitate in the organic phase was filtered off and washed with ethyl acetate. The solvent of the filtrate was removed and the residue was dried under high vacuum. This gave 4021 mg (62% of theory) of the title compound.

¹H NMR (400 MHz, DMSO-d₆): δ=ppm 0.68-0.89 (m, 2H), 1.17 (m, 3H), 1.37 (s, 9H), 1.66 (m, 4H), 2.02-2.15 (m, 1H), 2.74 (m, 3H), 2.93-3.07 (m, 1H), 3.98-4.09 (dd, 1H), 4.52-4.66 (dd, 1H), 6.72-6.88 (m, 2H), 7.02 (dd, 1H), 7.25 (d, 2H), 7.38-7.53 (m, 3H), 8.10 (d, 1H), 10.04 (s, 1H), 10.51 (s, 1H), 10.59 (s, 1H)

LC-MS (Method 1): Rₜ=1.00 min; MS (ESIneg): m/z=612 [M−H]⁻.

### Example 6A

### 5-{4-[(2S)-2-{[(trans-4-{[(tert-Butoxycarbonyl)amino]methyl}cyclohexyl)carbonyl]amino}-3-oxo-3-{[4-(2H-tetrazol-5-yl)phenyl]amino}propyl]phenyl}-6-methylpyridine-2-carboxylic acid

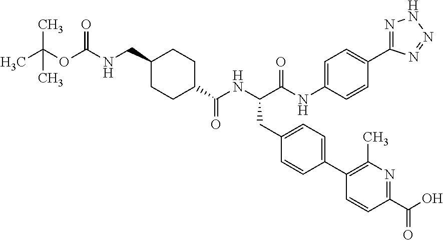

121.6 mg (0.48 mmol) of bis(pinacolato)diborane, 102.8 mg (0.45 mmol) of methyl 5-bromo-6-methylpyridine-2-carboxylate and 94.0 mg (0.96 mmol) of potassium acetate were initially charged in 2.5 ml of toluene under argon, 13.0 mg (0.016 mmol) of [1,1-bis(diphenylphosphine)ferrocene]-dichloropalladium-dichloromethane complex were added and the mixture was stirred at 110° C. for 3 h. The mixture was concentrated and dried under high vacuum. The residue was taken up in 3 ml of 1,2-dimethoxyethane and 1 ml of ethanol, and 200 mg (0.32 mmol) of 4-bromo-N-alpha-[(trans-4-{[(tert-butoxycarbonyl)amino]methyl}cyclohexyl)carbonyl]-N-[4-(2H-tetrazol-5-yl)phenyl]-L-phenylalaninamide, 13.0 mg (0.016 mmol) of 1,1-Bis-(diphenylphosphine)ferrocene]-dichloropalladium-dichloromethane complex and 0.32 ml (0.64 mmol) of 2M sodium carbonate solution in water were added. The mixture was stirred at RT for 16 h. The mixture was filtered through kieselguhr and the filtrate was separated by means of preparative HPLC (eluent: acetonitrile/water with 0.01% TFA (gradient)). The product-containing fractions were combined and concentrated on a rotary evaporator. The residue was dried under high vacuum. 47 mg (18% of theory, 85% purity) of the title compound were obtained.

¹H NMR (400 MHz, DMSO-d₆) δ=ppm 0.77-0.98 (m, 2H), 1.07-1.30 (m, 3H), 1.37 (s, 9H), 1.50-1.81 (m, 5H), 2.07-2.17 (m, 1H), 2.44 (s, 3H), 2.71-2.78 (m, 2H), 2.91-3.01 (m, 1H), 3.08-3.20 (m, 2H), 4.70-4.80 (m, 2H), 6.76-6.82 (m, 1H), 7.36 (br. d, 2H), 7.43 (br. d, 2H), 7.74 (d, 1H), 7.82 (d, 2H), 7.93 (d, 1H), 7.99 (d, 2H), 8.17-8.24 (m, 1H), 10.45 (s, 1H).

LC-MS (Method 1): Rₜ=0.82 min; MS (ESIpos): m/z=683 [M+H]⁺.

### Example 7A

### 5-{4-[(2S)-2-{[(trans-4-{[(tert-Butoxycarbonyl)amino]methyl}cyclohexyl)carbonyl]amino}-3-oxo-3-{[4-(1H-tetrazol-5-yl)phenyl]amino}propyl]phenyl}-4-methylpyridine-2-carboxylic acid

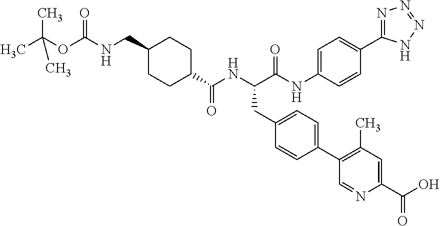

A solution of 4-bromo-N-alpha-[(trans-4-{[(tert-butoxycarbonyl)amino]methyl}cyclohexyl)-carbonyl]-N-[4-(2H-tetrazol-5-yl)phenyl]-L-phenylalaninamide (1884 mg, 3.0 mmol), methyl 4-methyl-5-(4,4,5,5-tetramethyl-1,3,2-dioxaborolan-2-yl)pyridine-2-carboxylate (1000 mg, 3.6 mmol) and tetrakis(triphenylphosphine)palladium(0) (347 mg, 0.3 mmol) in dimethylformamide (24 ml) was admixed with sodium carbonate (956 mg, 9.0 mmol) and water (4.5 ml) and heated at 110° C. in a microwave (Biotage Initiator) for 120 min. The reaction mixture was admixed with 1N sodium hydroxide solution (6 ml, 6.0 mmol) and stirred at 50° for 5 h and at RT overnight. Subsequently, acetonitrile was added and the precipitate formed was filtered off with suction and dried under high vacuum. This gave 2720 mg (100% of theory, 75% purity) of the title compound.

¹H NMR (300 MHz, DMSO-d₆): δ=ppm 0.70-0.87 (m, 2H), 1.17-1.25 (m, 2H), 1.33 (s, 9H), 1.44-1.70 (m, 7H), 1.88-2.00 (m, 1H), 2.02-2.14 (m, 1H), 2.19 (s, 3H), 2.91 (dd, 1H), 3.09 (dd, 1H), 4.67-4.77 (m, 1H), 6.71-6.78 (m, 1H), 7.25 (d, 2H), 7.37 (d, 2H), 7.56 (d, 2H), 7.81 (s, 1H), 7.85 (d, 2H), 7.92 (s, 1H), 8.09-8.19 (m, 2H), 10.12 (s, 1H)

LC-MS (Method 4): Rₜ=0.93 min; MS (ESIpos): m/z=683.5 [M+H]⁺.

### Example 8A

### 5-(4-{(2S)-2-{[(trans-4-{[(tert-Butoxycarbonyl)amino]methyl}cyclohexyl)carbonyl]amino}-3-oxo-3-[(3-oxo-2,3-dihydro-1H-indazol-6-yl)amino]propyl}phenyl)-4-methylpyridine-2-carboxylic acid

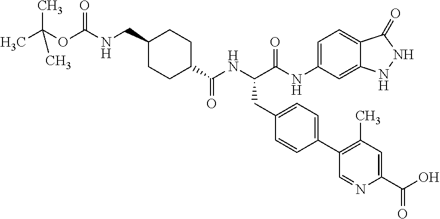

A solution of N-alpha-[(trans-4-{[(tert-butoxycarbonyl)amino]methyl}cyclohexyl)-carbonyl]-4-bromo-N-(3-oxo-2,3-dihydro-1H-indazol-6-yl)-L-phenylalaninamide (500 mg, 0.8 mmol), methyl 4-methyl-5-(4,4,5,5-tetramethyl-1,3,2-dioxaborolan-2-yl)pyridine-2-carboxylate (270.6 mg, 0.98 mmol) and tetrakis(triphenylphosphine)palladium(0) (94 mg, 0.08 mmol) in dimethylformamide (6.5 ml) was admixed with sodium carbonate (259 mg, 2.4 mmol) and water (1.22 ml) and heated at 110° C. in a microwave (Biotage Initiator) for 120 min. The reaction mixture was admixed with 1N sodium hydroxide solution (1.6 ml, 1.6 mmol) and stirred at RT overnight and at 50° C. for 2 h. Subsequently, acetonitrile was added and the precipitate formed was filtered off with suction and dried under high vacuum. This gave 699 mg (100% of theory, 80% purity) of the title compound.

¹H NMR (400 MHz, DMSO-d₆): δ=ppm 0.74-0.91 (m, 2H), 1.18-1.28 (m, 2H), 1.35 (s, 9H), 1.58-1.69 (m, 3H), 1.90-2.02 (m, 1H), 2.05-2.14 (m, 1H), 2.20 (s, 3H), 2.69-2.77 (m, 2H), 2.92 (dd, 1H), 3.08 (dd, 1H), 4.63-4.73 (m, 1H), 6.70-6.76 (m, 1H), 6.82 (d, 1H), 7.01 (dd, 1H), 7.16 (s, 1H), 7.25 (d, 2H), 7.36 (d, 2H), 7.42-7.45 (m, 1H), 7.77 (s, 1H), 8.11-8.18 (m, 2H), 9.99 (s, 1H)

LC-MS (Method 4): Rₜ=0.86 min; MS (ESIpos): m/z=671.4 [M+H]⁺.

### Example 9A

### N-[(trans-4-{[(tert-Butoxycarbonyl)amino]methyl}cyclohexyl)carbonyl]-4-[6-(ethoxycarbonyl)-2-methylpyridin-3-yl]-L-phenylalanine

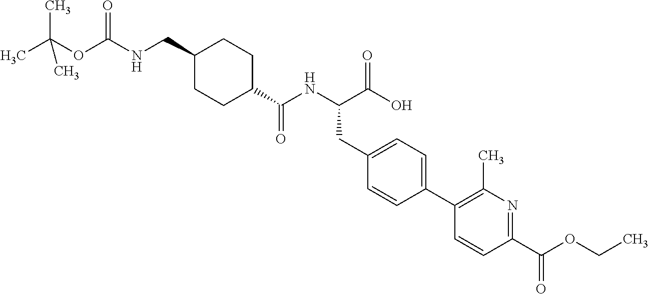

Methyl 5-bromo-6-methylpyridine-2-carboxylate (2769 mg, 12.03 mmol), bis(pinacolato)diborane (3274 mg, 12.9 mmol) and potassium acetate (2531 mg, 25.8 mmol) were initially charged in toluene (72 ml), purged with argon and then admixed with [1,1-bis-(diphenylphosphine)ferrocene]-dichloropalladium-dichloromethane complex (351 mg, 0.43 mmol). The reaction mixture was heated under reflux for 3 h and then concentrated. The residue was dried under high vacuum and then taken up in 1,2-dimethoxyethane (72 ml) and ethanol (29 ml). This was followed by the addition of 4-bromo-N-[(trans-4-[(tert-butoxycarbonyl)amino]methyl-cyclohexyl)carbonyl]-L-phenylalanine (4155 mg, 8.6 mmol), [1,1-bis-(diphenylphosphine)-ferrocene]-dichloropalladium-dichloromethane complex (351 mg, 0.43 mmol) and saturated aqueous sodium carbonate solution (8.6 ml). The reaction mixture was stirred under reflux for 6 h, concentrated and purified by chromatography by means of flash chromatography (ethyl acetate/ethanol gradient). This gave 2528 mg (38% of theory, 73% purity) of the title compound.

LC-MS (Method 1): Rₜ=1.0 min; MS (ESIpos): m/z=568.3 [M+H]⁺.

### Example 10A

### Ethyl 5-(4-{(2S)-2-{[(trans-4-{[(tert-butoxy carbonyl)amino]methyl}cyclohexyl)carbonyl]amino}-3-oxo-3-[(2-oxo-2,3-dihydro-1H-benzimidazol-5-yl)amino]propyl}phenyl)-6-methylpyridine-2-carboxylate

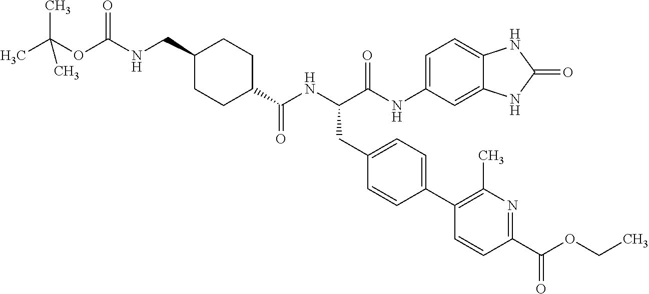

N-[(trans-4-{[(tert-Butoxycarbonyl)amino]methyl}cyclohexyl)carbonyl]-4-[6-(ethoxycarbonyl)-2-methylpyridin-3-yl]-L-phenylalanine (544 mg, 0.96 mmol) and 5-aminobenzimidazolone (286 mg, 1.9 mmol) were dissolved in dimethylformamide (6 ml), admixed with N-[(dimethylamino)(3H-[1,2,3]triazolo[4,5-b]pyridin-3-yloxy)methylidene]-N-methyl-methanaminium hexafluorophosphate (546 mg, 1.44 mmol) and stirred at RT overnight. The reaction mixture was admixed with a little water and acetonitrile, filtered through a Millipore filter and purified by chromatography by means of HPLC (eluent: acetonitrile/water with 0.1% TFA (gradient)). This gave 567 mg (85% of theory) of the title compound.

¹H NMR (400 MHz, DMSO-d₆): δ=ppm 0.71-0.89 (m, 2H), 1.05-1.17 (m, 1H), 1.23 (m, 2H), 1.37 (s, 9H), 1.47-1.56 (m, 1H), 1.58-1.71 (m, 3H), 2.05-2.16 (m, 1H), 2.45 (s, 3H), 2.74 (m, 2H), 2.93 (dd, 1H), 3.08 (dd, 1H), 4.36 (q, 2H), 4.70 (m, 1H), 6.74-6.81 (m, 1H), 6.84 (d, 1H), 7.01 (dd, 1H), 7.35 (d, 2H), 7.38-7.44 (m, 3H), 7.75 (d, 1H), 7.94 (d, 1H), 8.10 (d, 1H), 9.96 (s, 1H), 10.50 (s, 1H), 10.56 (s, 1H)

LC-MS (Method 1): Rₜ=0.98 min; MS (ESIpos): m/z=699.3 [M+H]⁺.

### Example 11A

### 5-(4-{(2S)-2-{[(trans-4-{[(tert-Butoxycarbonyl)amino]methyl}cyclohexyl)carbonyl]amino}-3-oxo-3-[(2-oxo-2,3-dihydro-1H-benzimidazol-5-yl)amino]propyl}phenyl)-6-methylpyridine-2-carboxylic acid

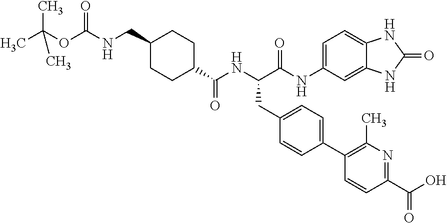

Ethyl 5-(4-{(2S)-2-{[(trans-4-{[(tert-butoxycarbonyl)amino]methyl}cyclohexyl)carbonyl]amino}-3-oxo-3-[(2-oxo-2,3-dihydro-1H-benzimidazol-5-yl)amino]propyl}phenyl)-6-methylpyridine-2-carboxylate (564 mg, 0.8 mmol) were taken up in tetrahydrofuran/water 3/1 (12 ml), admixed with lithium hydroxide monohydrate (339 mg, 8 mmol) and stirred at RT overnight. Subsequently, the solvent was decanted off from the solids, and the residue was washed with a little tetrahydrofuran, stirred with 0.5N sodium hydroxide solution, washed with a little water and dried under high vacuum. This gave 450 mg (83% of theory) of the title compound.

¹H NMR (400 MHz, DMSO-d₆): δ=ppm 0.73-0.89 (m, 2H), 1.07-1.17 (m, 1H), 1.17-1.30 (m, 2H), 1.36 (s, 9H), 1.49-1.57 (m, 1H), 1.58-1.72 (m, 3H), 2.05-2.16 (m, 1H), 2.35 (s, 3H), 2.74 (m, 2H), 2.91 (dd, 1H), 3.07 (dd, 1H), 4.64-4.72 (m, 1H), 6.75-6.79 (m, 1H), 6.82 (d, 1H), 7.00 (d, 1H), 7.28 (d, 2H), 7.36 (d, 2H), 7.43 (s, 1H), 7.49-7.55 (m, 1H), 7.71-7.79 (m, 1H), 8.12-8.20 (m, 1H), 9.99 (s, 1H), 10.54 (br. s, 2H)

LC-MS (Method 1): Rₜ=0.76 min; MS (ESIpos): m/z=671.3 [M+H]⁺.

### Example 12A

### N-alpha-[(trans-4-{[(tert-Butoxycarbonyl)amino]methyl}cyclohexyl)carbonyl]-4-[6-{[2-(diethylamino)ethyl]carbamoyl}-2-(trifluoromethyl)pyridin-3-yl]-N-[4-(2H-tetrazol-5-yl)phenyl]-L-phenylalaninamide trifluoroacetate

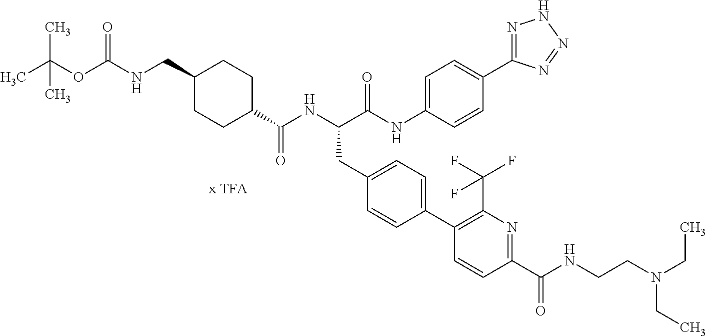

A solution of 80 mg (0.11 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-(trifluoromethyl)pyridine-2-carboxylic acid and 25.2 mg (0.22 mmol) of 2-diethylaminoethylamine in 1 ml of dimethylformamide was admixed with 57 μl (0.33 mmol) of N,N-diisopropylethylamine and 61.9 mg (0.16 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The reaction mixture was diluted with water and acetonitrile and separated by means of preparative HPLC (eluent: acetonitrile/water with 0.01% TFA (gradient)). The product-containing fractions were combined and concentrated on a rotary evaporator. The residue was dried under high vacuum. 47 mg (44% of theory) of the title compound were obtained.

LC-MS (Method 1): Rₜ=0.86 min; MS (ESIpos): m/z=835.4 [M+H-TFA]⁺.

### Example 13A

### N-alpha-[(trans-4-{[(tert-Butoxycarbonyl)amino]methyl}cyclohexyl)carbonyl]-4-(6-{[4-(dimethylamino)cyclohexyl]carbamoyl}-2-methylpyridin-3-yl)-N-[4-(2H-tetrazol-5-yl)phenyl]-L-phenylalaninamide trifluoroacetate

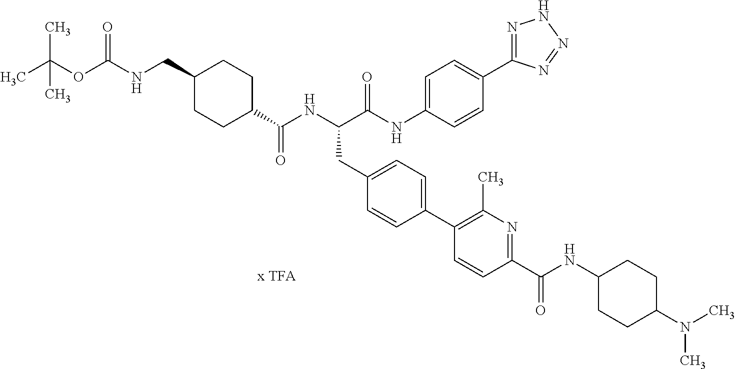

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 31.6 mg (0.29 mmol) of N,N-dimethylcyclohexane-1,4-diamine in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The reaction mixture was diluted with water and acetonitrile and separated by means of preparative HPLC (eluent: acetonitrile/water with 0.01% TFA (gradient)). The product-containing fractions were combined and concentrated on a rotary evaporator. The residue was dried under high vacuum. 94 mg (67% of theory) of the title compound were obtained.

LC-MS (Method 1): Rₜ=0.80 min; MS (ESIpos): m/z=808.4 [M+H-TFA]⁺.

### Example 14A

### N-alpha-[(trans-4-{[(tert-Butoxycarbonyl)amino]methyl}cyclohexyl)carbonyl]-4-(6-{[2-(diethylamino)ethyl]carbamoyl}-2-methylpyridin-3-yl)-N-[4-(2H-tetrazol-5-yl)phenyl]-L-phenylalaninamide trifluoroacetate

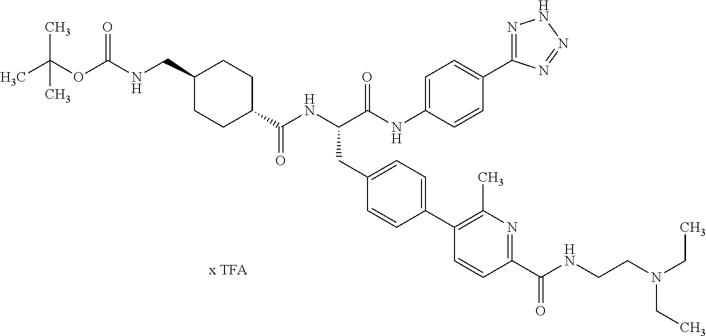 [SMILES: CCN(CC)CCNC(=O)c1ccc(-c2ccc(C[C@H](NC(=O)[C@H]3CC[C@H](CNC(=O)OC(C)(C)C)CC3)C(=O)Nc3ccc(-c4nn[nH]n4)cc3)cc2)c(C)n1]

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 38.1 mg (0.29 mmol) of 2-diethylaminoethylamine in 1.25 ml of dimethylformamide was admixed with 77 al (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The reaction mixture was diluted with water and acetonitrile and separated by means of preparative HPLC (eluent: acetonitrile/water with 0.01% TFA (gradient)). The product-containing fractions were combined and concentrated on a rotary evaporator. The residue was dried under high vacuum. 90 mg (61% of theory, 88% purity) of the title compound were obtained.

LC-MS (Method 1): Rₜ=0.80 min; MS (ESIpos): m/z=782.4 [M+H-TFA]⁺.

### Example 15A

### N-alpha-[(trans-4-{[(tert-Butoxycarbonyl)amino]methyl}cyclohexyl)carbonyl]-4-(2-methyl-6-{[(3S)-2-oxopiperidin-3-yl]carbamoyl}pyridin-3-yl)-N-[4-(2H-tetrazol-5-yl)phenyl]-L-phenylalaninamide trifluoroacetate

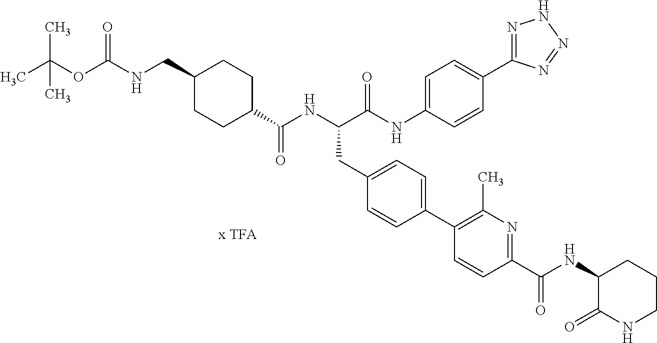

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 33.4 mg (0.29 mmol) of (3S)-3-aminopiperidin-2-one in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 16 h. The reaction mixture was diluted with water and acetonitrile and separated by means of preparative HPLC (eluent: acetonitrile/water with 0.01% TFA (gradient)). The product-containing fractions were combined and concentrated on a rotary evaporator. The residue was dried under high vacuum. 58 mg (34% of theory, 78% purity) of the title compound were obtained.

LC-MS (Method 1): Rₜ=0.92 min; MS (ESIpos): m/z=780.3 [M+H-TFA]⁺.

### Example 16A

### tert-Butyl 4-{[(5-{4-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]phenyl}-4-methylpyridin-2-yl)carbonyl]amino}piperidine-1-carboxylate formate

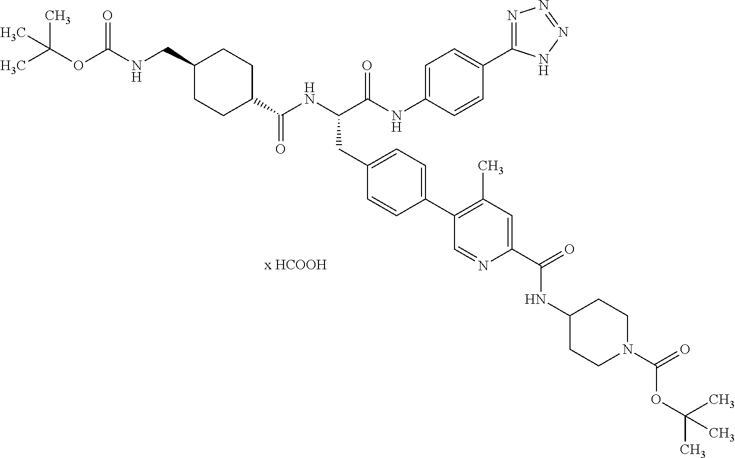

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 88.0 mg (0.44 mmol) of tert-butyl 4-aminopiperidine-1-carboxylate in 2 ml of dimethylformamide was admixed with 115 μl (0.66 mmol) of N,N-diisopropylethylamine and 125.3 mg (0.33 mmol) of N-[(dimethylamino)(3H-[1,2,3]triazolo[4,5-b]pyridin-3-yloxy)methylidene]-N-methylmethanaminium hexafluorophosphate and stirred at RT for 24 h. Subsequently, the mixture was stirred at 40° C. for 2 h, then a further 0.5 eq. of N-[(dimethylamino)(3H-[1,2,3]triazolo[4,5-b]pyridin-3-yloxy)methylidene]-N-methylmethanaminium hexafluorophosphate was added and the mixture was stirred at RT overnight. The reaction mixture was filtered and the filtrate was separated by means of preparative HPLC (Method 10). 33 mg (17% of theory) of the title compound were obtained.

LC-MS (Method 4): Rₜ=1.33 min; MS (ESIpos): m/z=865.5 [M+H—HCOOH]⁺.

### Example 17A

### N-alpha-[(trans-4-{[(tert-Butoxycarbonyl)amino]methyl}cyclohexyl)carbonyl]-4-{6-[(3,5-dimethylpiperazin-1-yl)carbonyl]-4-methylpyridin-3-yl}-N-[4-(1H-tetrazol-5-yl)phenyl]-L-phenylalaninamide

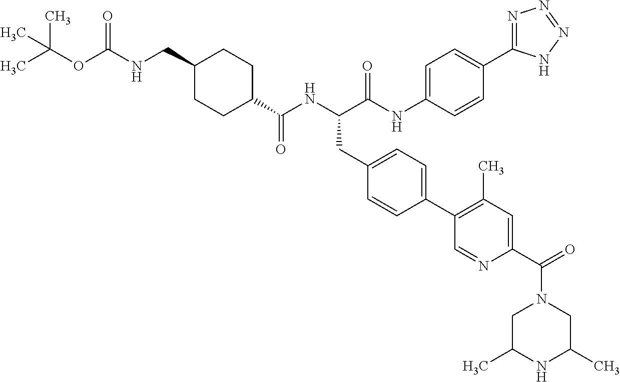

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 50.2 mg (0.44 mmol) of 2,6-dimethylpiperazine in 2 ml of dimethyl sulphoxide was admixed with 115 μl (0.66 mmol) of N,N-diisopropylethylamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for 4 h. The reaction mixture was filtered and separated by means of preparative HPLC (Method 11). 27 mg (16% of theory) of the title compound were obtained.

LC-MS (Method 5): Rₜ=0.77 min; MS (ESIpos): m/z=779.6 [M+H]⁺.

### Example 18A

### N-alpha-[(trans-4-{[(tert-Butoxycarbonyl)amino]methyl}cyclohexyl)carbonyl]-4-(6-{[4-(diethylamino)cyclohexyl]carbamoyl}-4-methylpyridin-3-yl)-N-[4-(1H-tetrazol-5-yl)phenyl]-L-phenylalaninamide

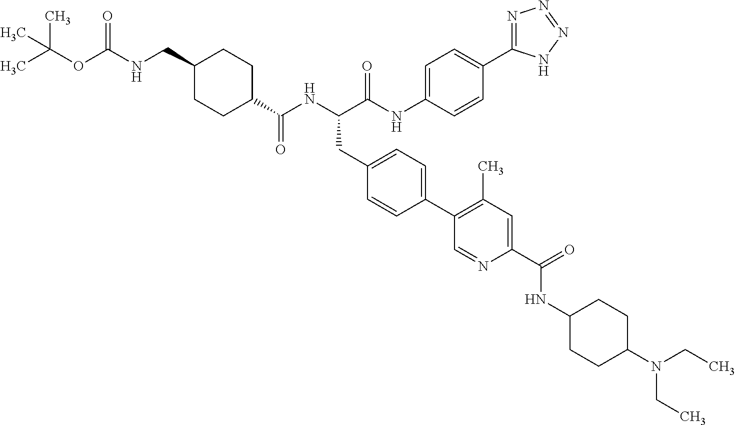

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 74.8 mg (0.44 mmol) of N,N-diethylcyclohexane-1,4-diamine in 2 ml of dimethyl sulphoxide was admixed with 115 μl (0.66 mmol) of N,N-diisopropylethylamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for 4 h. The reaction mixture was filtered and the filtrate was separated by means of preparative HPLC (Method 11). 26 mg (14% of theory) of the title compound were obtained.

LC-MS (Method 5): Rₜ=0.94 min; MS (ESIpos): m/z=835.6 [M+H]⁺.

### Example 19A

### tert-Butyl 4-{[(5-{4-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]phenyl}-4-methylpyridin-2-yl)carbonyl]amino}-3-fluoropiperidine-1-carboxylate

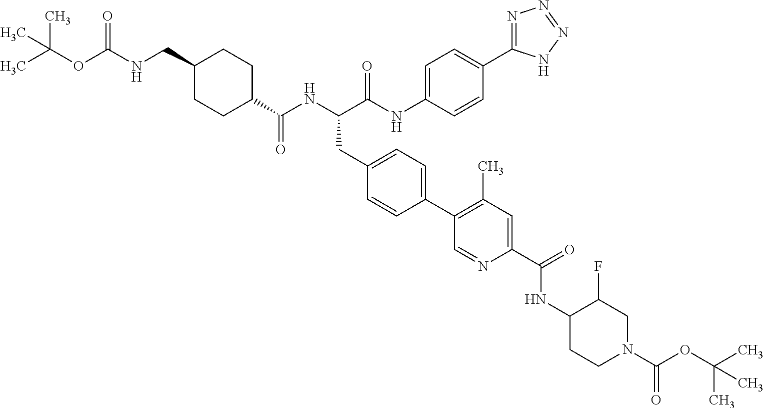

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 95.9 mg (0.44 mmol) of tert-butyl 4-amino-3-fluoropiperidine-1-carboxylate in 2 ml of dimethylformamide was admixed with 115 μl (0.66 mmol) of N,N-diisopropylethylamine and 125.3 mg (0.33 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for 4 h. The reaction mixture was filtered and the filtrate was separated by means of preparative HPLC (Method 11). 13 mg (7% of theory) of the title compound were obtained.

LC-MS (Method 5): Rₜ=0.96 min; MS (ESIpos): m/z=883.5 [M+H]⁺.

### Example 20A

### N-alpha-[(trans-4-{[(tert-Butoxycarbonyl)amino]methyl}cyclohexyl)carbonyl]-4-(6-{[3-(diethylamino)propyl]carbamoyl}-4-methylpyridin-3-yl)-N-[4-(1H-tetrazol-5-yl)phenyl]-L-phenylalaninamide

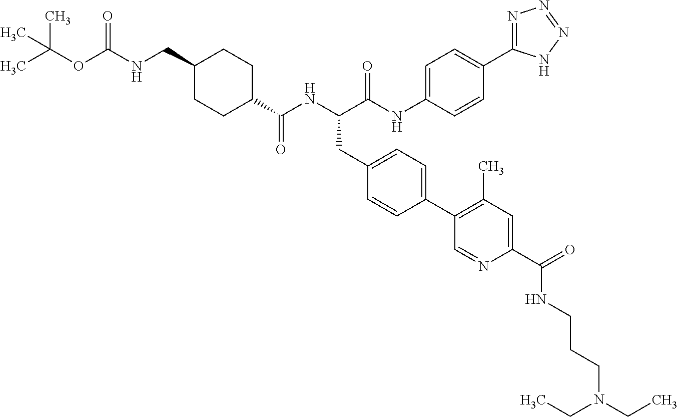

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 57.2 mg (0.44 mmol) of N,N-diethylpropane-1,3-diamine in 2 ml of dimethyl sulphoxide was admixed with 115 μl (0.66 mmol) of N,N-diisopropylethylamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for 4 h. The reaction mixture was filtered and the filtrate was separated by means of preparative HPLC (Method 11). 19 mg (11% of theory) of the title compound were obtained.

LC-MS (Method 5): Rₜ=0.90 min; MS (ESIpos): m/z=795.6 [M+H]⁺.

### Example 21A

### tert-Butyl (1R,5S)-3-{[(5-{4-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]phenyl}-4-methylpyridin-2-yl)carbonyl]amino}-8-azabicyclo[3.2.1]octane-8-carboxylate formate

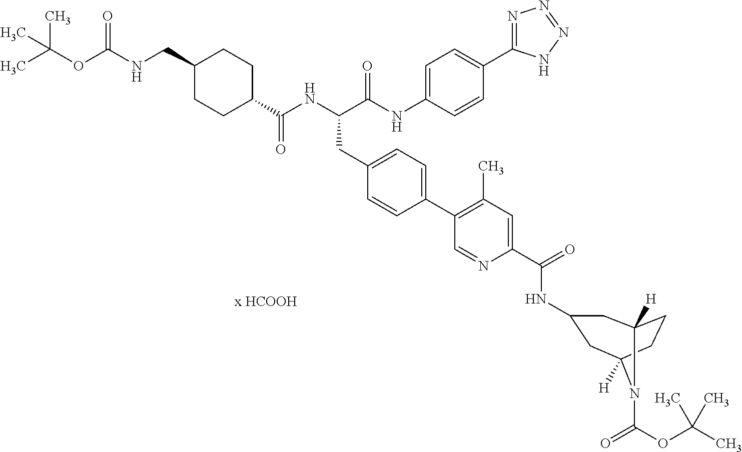 [SMILES: Cc1cc(C(=O)NC2C[C@H]3CC[C@@H](C2)N3C(=O)OC(C)(C)C)ncc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 99.4 mg (0.44 mmol) of tert-butyl (1R,5S)-3-amino-8-azabicyclo[3.2.1]octane-8-carboxylate in 2 ml of dimethylformamide was admixed with 115 μl (0.66 mmol) of N,N-diisopropylethylamine and 125.3 mg (0.33 mmol) of N-[(dimethylamino)(3H-[1,2,3]triazolo[4,5-b]pyridin-3-yloxy)methylidene]-N-methylmethanaminium hexafluorophosphate and stirred at RT for 24 h. Subsequently, a further 0.5 eq. of N-[(dimethylamino)(3H-[1,2,3]triazolo[4,5-b]pyridin-3-yloxy)methylidene]-N-methylmethanaminium hexafluorophosphate were added and the mixture was stirred at 40° C. for 4 h. The reaction mixture was filtered and the filtrate was separated by means of preparative HPLC (Method 15). 33 mg (17% of theory) of the title compound were obtained.

LC-MS (Method 5): Rₜ=0.99 min; MS (ESIpos): m/z=891.6 [M+H—HCOOH]⁺.

### Example 22A

### tert-Butyl 7-{[(5-{4-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]phenyl}-4-methylpyridin-2-yl)carbonyl]amino}-3-oxa-9-azabicyclo[3.3.1]nonane-9-carboxylate formate

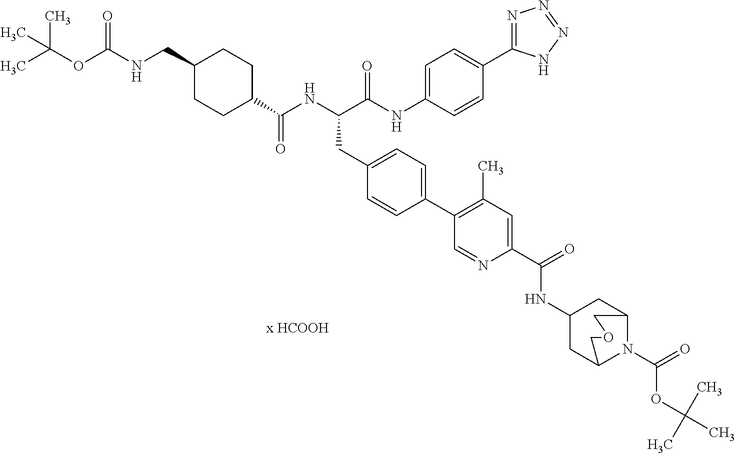

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 106.5 mg (0.44 mmol) of tert-butyl 7-amino-3-oxa-9-azabicyclo[3.3.1]nonane-9-carboxylate in 2 ml of dimethylformamide was admixed with 115 (0.66 mmol) of N,N-diisopropylethylamine and 125.3 mg (0.33 mmol) of N-[(dimethylamino)(3H-[1,2,3]triazolo[4,5-b]pyridin-3-yloxy)methylidene]-N-methylmethanaminium hexafluorophosphate and stirred at RT for 24 h. After addition of a further 0.5 eq. of N-[(dimethylamino)(3H-[1,2,3]triazolo[4,5-b]pyridin-3-yloxy)methylidene]-N-methylmethanaminium hexafluorophosphate, the mixture was stirred at 40° C. for 4 h. The reaction mixture was filtered and the filtrate was separated by means of preparative HPLC (Method 10). 33 mg (16% of theory) of the title compound were obtained.

LC-MS (Method 1): Rₜ=1.32 min; MS (ESIpos): m/z=907.6 [M+H—HCOOH]⁺.

### Example 23A

### N-alpha-[(trans-4-{[(tert-Butoxycarbonyl)amino]methyl}cyclohexyl)carbonyl]-4-(6-{[3-(dimethylamino)propyl]carbamoyl}-4-methylpyridin-3-yl)-N-[4-(1H-tetrazol-5-yl)phenyl]-L-phenylalaninamide

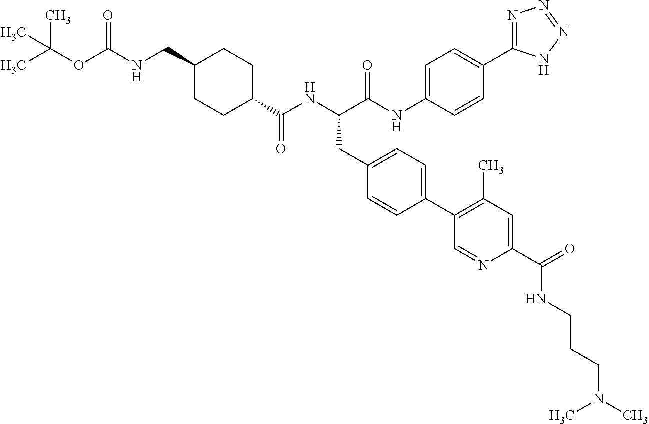 [SMILES: Cc1cc(C(=O)NCCCN(C)C)ncc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 44.9 mg (0.44 mmol) of N,N-dimethylpropane-1,3-diamine in 2 ml of dimethyl sulphoxide was admixed with 115 μl (0.66 mmol) of N,N-diisopropylethylamine-1,3-diamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for 4 h. The reaction mixture was filtered and separated by means of preparative HPLC (Method 11). 14 mg (8% of theory) of the title compound were obtained.

LC-MS (Method 5): Rₜ=0.84 min; MS (ESIpos): m/z=767.5 [M+H]⁺.

### Example 24A

### N-alpha-[(trans-4-{[(tert-Butoxycarbonyl)amino]methyl}cyclohexyl)carbonyl]-4-(4-methyl-6-{[trans-4-(morpholin-4-yl)cyclohexyl]carbamoyl}pyridin-3-yl)-N-[4-(1H-tetrazol-5-yl)phenyl]-L-phenylalaninamide

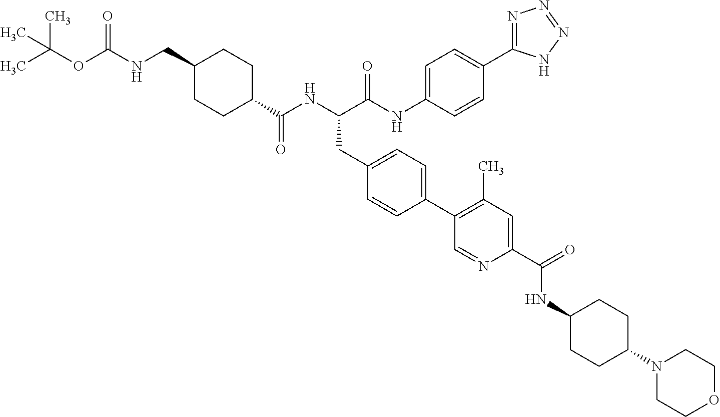 [SMILES: Cc1cc(C(=O)N[C@H]2CC[C@H](N3CCOCC3)CC2)ncc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 80.97 mg (0.44 mmol) of trans-4-(morpholin-4-yl)cyclohexanamine in 2 ml of dimethylformamide was admixed with 115 μl (0.66 mmol) of N,N-diisopropylethylamine and 125.3 mg (0.33 mmol) of N-[(dimethylamino)(3H-[1,2,3]triazolo[4,5-b]pyridin-3-yloxy)methylidene]-N-methylmethanaminium hexafluorophosphate and stirred at RT for 24 h. After addition of a further 0.5 eq. of N-[(dimethylamino)(3H-[1,2,3]triazolo[4,5-b]pyridin-3-yloxy)methylidene]-N-methylmethanaminium hexafluorophosphate, the mixture was stirred at 40° C. for 4 h. The reaction mixture was filtered and the filtrate was separated by means of preparative HPLC (Method 11). 20 mg (11% of theory) of the title compound were obtained.

LC-MS (Method 5): Rₜ=0.86 min; MS (ESIpos): m/z=849.5 [M+H]⁺.

### Example 25A

### tert-Butyl (3S)-3-({[5-(4-{(2S)-2-{[(trans-4-{[(tert-butoxycarbonyl)amino]methyl}cyclohexyl)-carbonyl]amino}-3-oxo-3-[(2-oxo-2,3-dihydro-1H-benzimidazol-5-yl)amino]propyl}phenyl)-6-methylpyridin-2-yl]carbonyl}amino)pyrrolidine-1-carboxylate trifluoroacetate

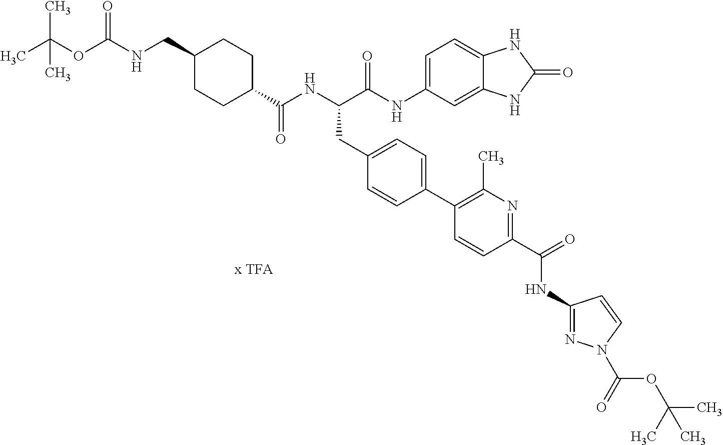

A solution of 80 mg (0.12 mmol) of 5-(4-{(2S)-2-{[(trans-4-{[(tert-butoxycarbonyl)amino]-methyl}cyclohexyl)carbonyl]amino}-3-oxo-3-[(2-oxo-2,3-dihydro-1H-benzimidazol-5-yl)-amino]propyl}phenyl)-6-methylpyridine-2-carboxylic acid and 44.4 mg (0.24 mmol) of tert-butyl (3S)-3-aminopyrrolidine-1-carboxylate in 1 ml of dimethylformamide was admixed with 62 μl (0.36 mmol) of N,N-diisopropylethylamine and 68.0 mg (0.18 mmol) of N-[(dimethylamino)(3H-[1,2,3]triazolo[4,5-b]pyridin-3-yloxy)methylidene]-N-methylmethanaminium hexafluorophosphate and stirred at RT for 24 h. After addition of 1 eq. of N-[(dimethylamino)(3H-[1,2,3]triazolo[4,5-b]pyridin-3-yloxy)methylidene]-N-methylmethanaminium hexafluorophosphate and 1 eq. of N,N-diisopropylethylamine, the mixture was stirred at 50° C. for 2 h. The reaction mixture was filtered and the filtrate was separated by means of preparative HPLC (eluent: acetonitrile/water with 0.01% TFA (gradient)). 38 mg (38% of theory) of the title compound were obtained.

LC-MS (Method 1): Rₜ=1.07 min; MS (ESIpos): m/z=839.5 [M+H-TFA]⁺.

### Example 26A

### tert-Butyl (3S)-3-{[(5-{4-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]phenyl}-6-methylpyridin-2-yl)carbonyl]amino}pyrrolidine-1-carboxylate trifluoroacetate

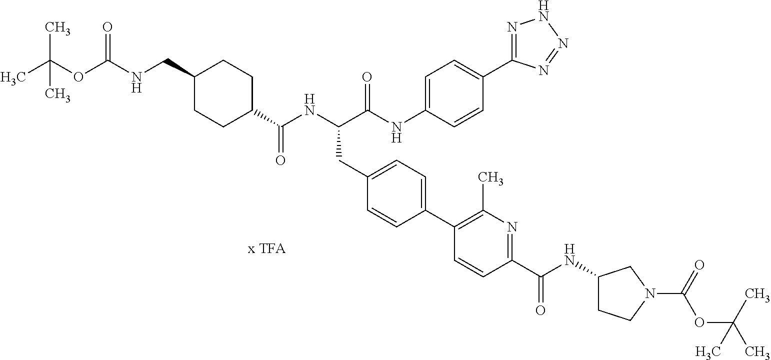

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 54.6 mg (0.29 mmol) of tert-butyl (3S)-3-aminopyrrolidine-1-carboxylate in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 77 mg (46% of theory) of the title compound were obtained.

LC-MS (Method 1): Rₜ=1.13 min; MS (ESIpos): m/z=851.6 [M+H-TFA]⁺.

### Example 27A

### tert-Butyl 6-{[(5-{4-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]phenyl}-6-methylpyridin-2-yl)carbonyl]amino}-3-azabicyclo[3.1.0]hexane-3-carboxylate trifluoroacetate

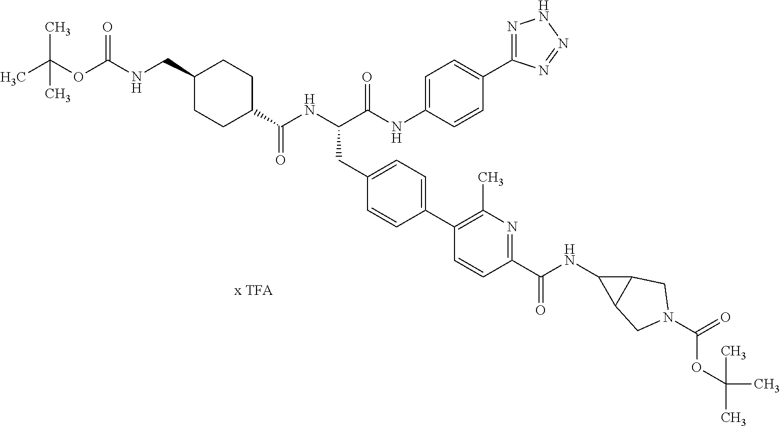

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 58.1 mg (0.22 mmol) of tert-butyl 6-amino-3-azabicyclo[3.1.0]hexane-3-carboxylate in 1.25 ml of dimethylformamide was admixed with 77 (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 90 mg (57% of theory) of the title compound were obtained.

LC-MS (Method 1): Rₜ=1.14 min; MS (ESIpos): m/z=863.4 [M+H-TFA]⁺.

### Example 28A

### tert-Butyl (3R)-3-{[(5-{4-[(2S)-2-{[(trans-4-{[(tert-butoxycarbonyl)amino]methyl}cyclohexyl)-carbonyl]amino}-3-oxo-3-{[4-(2H-tetrazol-5-yl)phenyl]amino}propyl]phenyl}-6-methylpyridin-2-yl)carbonyl]amino}pyrrolidine-1-carboxylate trifluoroacetate

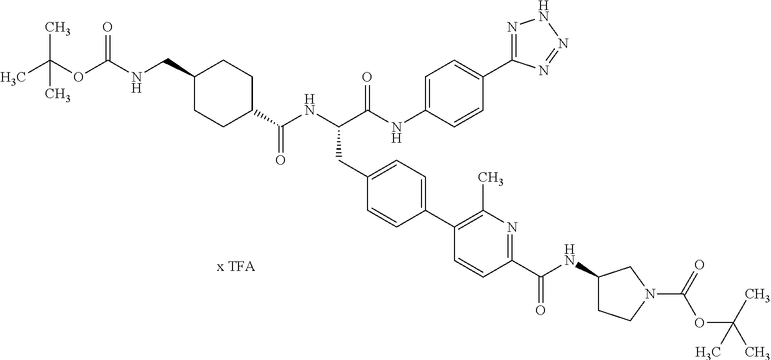

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 54.6 mg (0.29 mmol) of tert-butyl (3R)-3-aminopyrrolidine-1-carboxylate in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 77 mg (52% of theory) of the title compound were obtained.

LC-MS (Method 1): Rₜ=1.13 min; MS (ESIpos): m/z=851.5 [M+H-TFA]⁺.

### Example 29A

### tert-Butyl (2R,4R)-4-{[(5-{4-[(2S)-2-{[(trans-4-{[(tert-butoxycarbonyl)amino]methyl}-cyclohexyl)carbonyl]amino}-3-oxo-3-{[4-(2H-tetrazol-5-yl)phenyl]amino}propyl]phenyl}-6-methylpyridin-2-yl)carbonyl]amino}-2-methylpiperidine-1-carboxylate trifluoroacetate

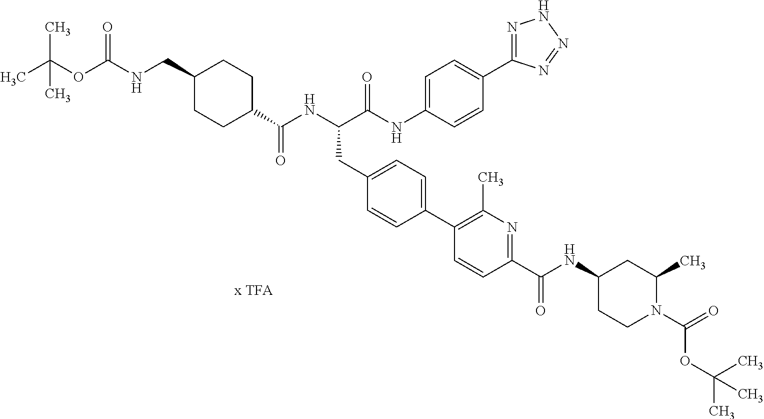

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 62.8 mg (0.29 mmol) of tert-butyl (2R,4R)-4-amino-2-methylpiperidine-1-carboxylate in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 90 mg (59% of theory) of the title compound were obtained.

LC-MS (Method 1): Rₜ=1.22 min; MS (ESIpos): m/z=879.5 [M+H-TFA]⁺.

### Example 30A

### N-alpha-[(trans-4-{[(tert-Butoxycarbonyl)amino]methyl}cyclohexyl)carbonyl]-4-(2-methyl-6-{[(3R)-1-methylpyrrolidin-3-yl]carbamoyl}pyridin-3-yl)-N-[4-(2H-tetrazol-5-yl)phenyl]-L-phenylalaninamide trifluoroacetate

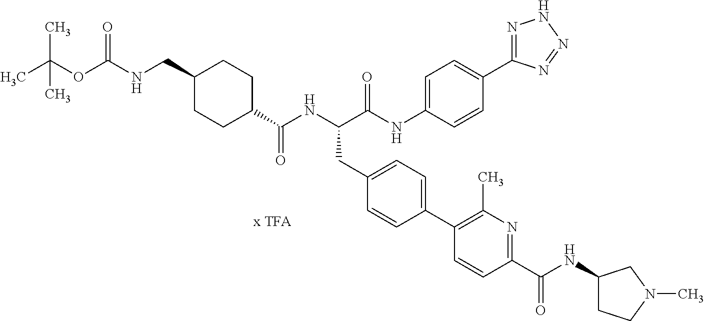

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 50.7 mg (0.29 mmol) of 1-methylpyrrolidin-3-amine dihydrochloride in 1.25 ml of dimethylformamide was admixed with 128 μl (0.73 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 51 mg (6% of theory, 15% purity) of the title compound were obtained. Partial cleavage of the tert-butoxycarbonyl group during chromatography.

LC-MS (Method 1): Rₜ=0.79 min; MS (ESIpos): m/z=765.4 [M+H-TFA]⁺.

### Example 31A

### tert-Butyl 5-{[(5-{4-[(2S)-2-{[(trans-4-{[(tert-butoxycarbonyl)amino]methyl}cyclohexyl)-carbonyl]amino}-3-oxo-3-{[4-(1H-tetrazol-5-yl)phenyl]amino}propyl]phenyl}-4-methylpyridin-2-yl)carbonyl]amino}-3,3-difluoropiperidine-1-carboxylate

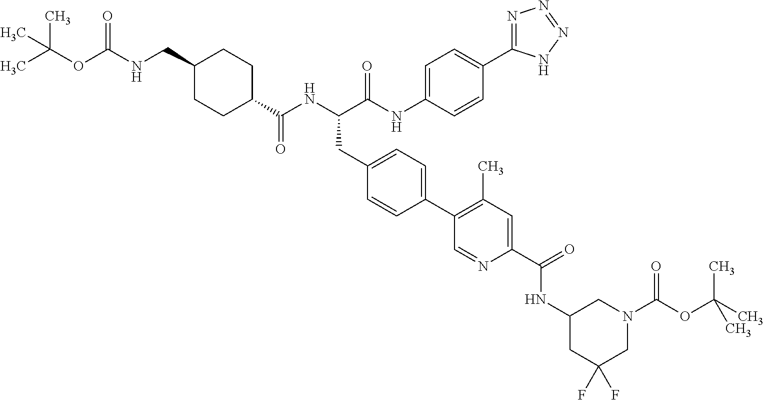 [SMILES: Cc1cc(C(=O)NC2CN(C(=O)OC(C)(C)C)CC(F)(F)C2)ncc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 103.8 mg (0.44 mmol) of tert-butyl 5-amino-3,3-difluoropiperidine-1-carboxylate in 2 ml of dimethyl sulphoxide was admixed with 115 μl (0.66 mmol) of N,N-diisopropylethylamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for a further 4 h. The reaction mixture was filtered and the filtrate was separated by means of preparative HPLC (Method 11). 16 mg (8% of theory) of the title compound were obtained.

LC-MS (Method 5): Rₜ=0.95 min; MS (ESIpos): m/z=901.6 [M+H]⁺.

### Example 32A

### N-alpha-[(trans-4-{[(tert-Butoxycarbonyl)amino]methyl}cyclohexyl)carbonyl]-4-(4-methyl-6-{[(3R)-2-oxopiperidin-3-yl]carbamoyl}pyridin-3-yl)-N-[4-(1H-tetrazol-5-yl)phenyl]-L-phenylalaninamide

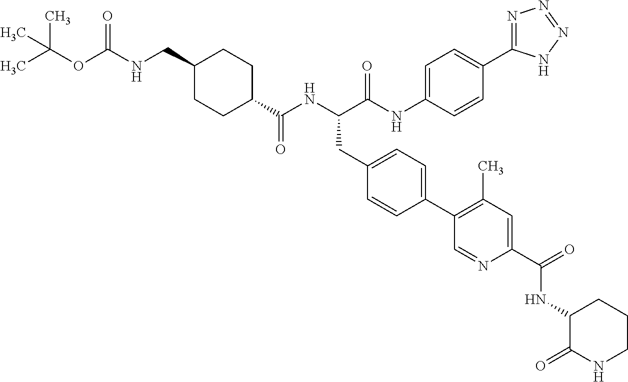

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 50.2 mg (0.44 mmol) of (3R)-3-aminopiperidin-2-one in 2 ml of dimethyl sulphoxide was admixed with 115 μl (0.66 mmol) of N,N-diisopropylethylamine and 125.3 mg (0.33 mmol) of N-[(dimethylamino) (3H-[1,2,3]triazolo[4,5-b]pyridin-3-yloxy)methylidene]-N-methylmethanaminium hexafluorophosphate and stirred at RT for 24 h. After addition of a further 0.5 eq. of N-[(dimethylamino)(3H-[1,2,3]triazolo[4,5-b]pyridin-3-yloxy)methylidene]-N-methylmethanaminium hexafluorophosphate, the mixture was stirred at 40° C. for 4 h. The reaction mixture was filtered and separated by means of preparative HPLC (Method 11). 16 mg (9% of theory) of the title compound were obtained.

LC-MS (Method 5): Rₜ=0.80 min; MS (ESIpos): m/z=779.4 [M+H]⁺.

### Example 33A

### N-alpha-[(trans-4-{[(tert-Butoxycarbonyl)amino]methyl}cyclohexyl)carbonyl]-4-{6-[(4-hydroxycyclohexyl)carbamoyl]-2-methylpyridin-3-yl}-N-[4-(2H-tetrazol-5-yl)phenyl]-L-phenylalaninamide trifluoroacetate

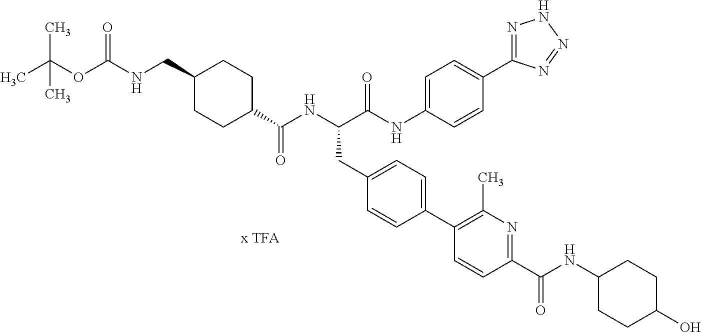

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 33.7 mg (0.29 mmol) of trans-4-aminocyclohexanol in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 61 mg (43% of theory, 93% purity) of the title compound were obtained.

LC-MS (Method 1): Rₜ=0.94 min; MS (ESIpos): m/z=780.4 [M+H-TFA]⁺.

### Example 34A

### N-alpha-[(trans-4-{[(tert-Butoxycarbonyl)amino]methyl}cyclohexyl)carbonyl]-4-{4-methyl-6-[(1-methyl-1H-pyrazol-3-yl)carbamoyl]pyridin-3-yl}-N-[4-(1H-tetrazol-5-yl)phenyl]-L-phenylalaninamide formate

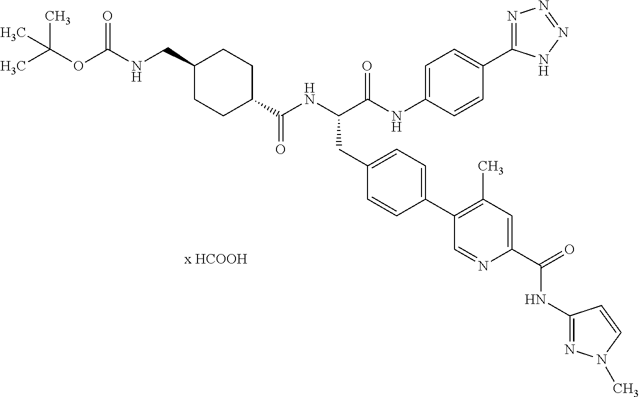 [SMILES: Cc1cc(C(=O)Nc2ccn(C)n2)ncc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 42.7 mg (0.44 mmol) of 1-methyl-1H-pyrazol-3-amine in 2 ml of dimethyl sulphoxide was admixed with 115 μl (0.66 mmol) of N,N-diisopropylethylamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (Method 10). 20 mg (27% of theory) of the title compound were obtained.

LC-MS (Method 4): Rₜ=1.19 min; MS (ESIpos): m/z=762.4 [M+H—HCOOH]⁺.

### Example 35A

### N-alpha-[(trans-4-{[(tert-Butoxycarbonyl)amino]methyl}cyclohexyl)carbonyl]-4-(6-{[3-(diethylamino)propyl]carbamoyl}-2-methylpyridin-3-yl)-N-[4-(2H-tetrazol-5-yl)phenyl]-L-phenylalaninamide trifluoroacetate

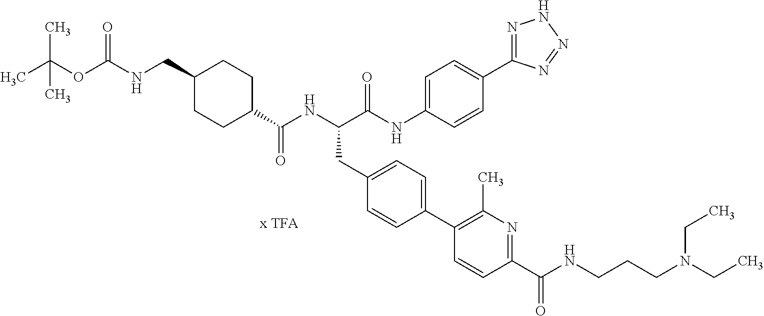

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 38.1 mg (0.29 mmol) of N,N-diethylpropane-1,3-diamine in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 39 mg (29% of theory) of the title compound were obtained.

LC-MS (Method 1): Rₜ=0.81 min; MS (ESIpos): m/z=795.5 [M+H-TFA]⁺.

### Example 36A

### N-alpha-[(trans-4-{[(tert-Butoxycarbonyl)amino]methyl}cyclohexyl)carbonyl]-4-{4-methyl-6-[(3-methylpiperazin-1-yl)carbonyl]pyridin-3-yl}-N-[4-(1H-tetrazol-5-yl)phenyl]-L-phenylalaninamide formate

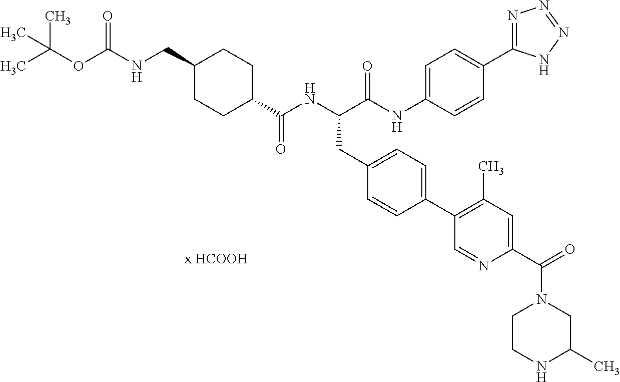 [SMILES: Cc1cc(C(=O)N2CCNC(C)C2)ncc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 44.0 mg (0.44 mmol) of 2-methylpiperazine in 2 ml of dimethyl sulphoxide was admixed with 115 μl (0.66 mmol) of N,N-diisopropylethylamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for a further 4 h. The reaction mixture was filtered and the filtrate was separated by means of preparative HPLC (Method 10). 9 mg (5% of theory) of the title compound were obtained.

LC-MS (Method 4): Rₜ=0.91 min; MS (ESIpos): m/z=765.6 [M+H—HCOOH]⁺.

### Example 37A

### N-alpha-[(trans-4-{[(tert-Butoxycarbonyl)amino]methyl}cyclohexyl)carbonyl]-4-(2-methyl-6-{[(3R)-2-oxopyrrolidin-3-yl]carbamoyl}pyridin-3-yl)-N-[4-(2H-tetrazol-5-yl)phenyl]-L-phenylalaninamide trifluoroacetate

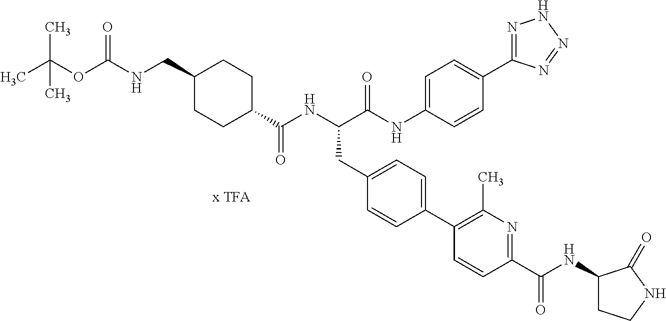 [SMILES: Cc1nc(C(=O)N[C@@H]2CCNC2=O)ccc1-c1ccc(C[C@H](NC(=O)[C@H]2CC[C@H](CNC(=O)OC(C)(C)C)CC2)C(=O)Nc2ccc(-c3nn[nH]n3)cc2)cc1]

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 29.3 mg (0.29 mmol) of (S)-3-aminopyrrolidin-2-one in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 68 mg (48% of theory) of the title compound were obtained.

LC-MS (Method 1): Rₜ=0.90 min; MS (ESIpos): m/z=765.4 [M+H-TFA]⁺.

### Example 38A

### N-alpha-[(trans-4-{[(tert-Butoxycarbonyl)amino]methyl}cyclohexyl)carbonyl]-4-(6-{[4-(dimethylamino)cyclohexyl]carbamoyl}-2-methylpyridin-3-yl)-N-(2-oxo-2,3-dihydro-1H-benzimidazol-5-yl)-L-phenylalaninamide trifluoroacetate

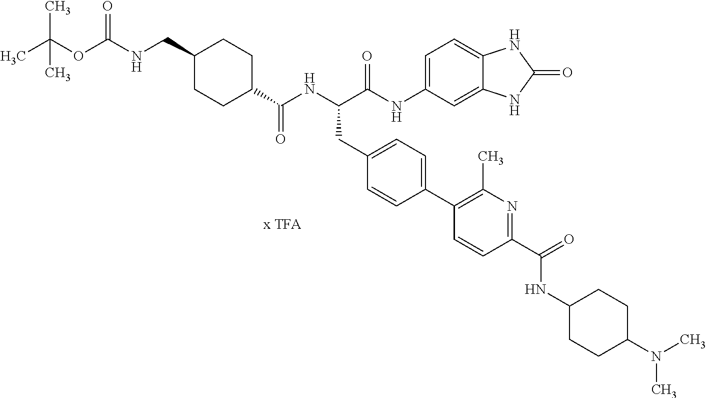

A solution of 80 mg (0.12 mmol) of 5-(4-{(2S)-2-{[(trans-4-{[(tert-butoxycarbonyl)amino]-methyl}cyclohexyl)carbonyl]amino}-3-oxo-3-[(2-oxo-2,3-dihydro-1H-benzimidazol-5-yl)-amino]propyl}phenyl)-6-methylpyridine-2-carboxylic acid and 33.9 mg (0.24 mmol) of N,N-dimethylcyclohexane-1,4-diamine in 1 ml of dimethylformamide was admixed with 62 μl (0.36 mmol) of N,N-diisopropylethylamine and 68.0 mg (0.18 mmol) of N-[(dimethylamino)(3H-[1,2,3]triazolo[4,5-b]pyridin-3-yloxy)methylidene]-N-methylmethanaminium hexafluorophosphate and stirred at RT for 24 h. After addition of 45 mg (0.12 mmol) of N-[(dimethylamino)(3H-[1,2,3]triazolo[4,5-b]pyridin-3-yloxy)methylidene]-N-methylmethanaminium hexafluorophosphate and 21 μl (0.12 mmol) of N,N-diisopropylethylamine, the mixture was stirred at 50° C. for 2 h. The reaction mixture was filtered and the filtrate was separated by means of preparative HPLC (eluent: acetonitrile/water with 0.1% TFA (gradient)). The product-containing fractions were combined, acetonitrile was distilled off and the aqueous phase was freeze-dried. The residue was taken up in a little dimethyl sulphoxide and acetonitrile and purified again by means of preparative HPLC. 23 mg (19% of theory) of the title compound were obtained.

LC-MS (Method 1): Rₜ=0.78 min; MS (ESIpos): m/z=795.5 [M+H-TFA]⁺.

### Example 39A

### N-alpha-[(trans-4-{[(tert-Butoxycarbonyl)amino]methyl}cyclohexyl)carbonyl]-4-(4-methyl-6-{[(3R)-2-oxopiperidin-3-yl]carbamoyl}pyridin-3-yl)-N-(2-oxo-2,3-dihydro-1H-benzimidazol-5-yl)-L-phenylalaninamide

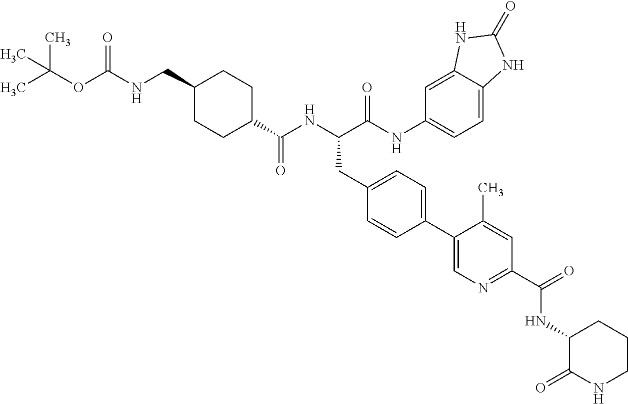

A solution of 175 mg (0.21 mmol) of 5-(4-{(2S)-2-{[(trans-4-{[(tert-butoxycarbonyl)amino]-methyl}cyclohexyl)carbonyl]amino}-3-oxo-3-[(2-oxo-2,3-dihydro-1H-benzimidazol-5-yl)amino]-propyl}phenyl)-4-methylpyridine-2-carboxylic acid and 49.6 mg (0.42 mmol) of (3R)-3-aminopiperidin-2-one in 1.9 ml of dimethyl sulphoxide was admixed with 109 μl (0.63 mmol) of N,N-diisopropylethylamine and 158.7 mg (0.42 mmol) of N-[(dimethylamino)(3H-[1,2,3]triazolo[4,5-b]pyridin-3-yloxy)methylidene]-N-methylmethanaminium hexafluorophosphate and stirred at RT for 24 h. After addition of 1 eq. of N-[(dimethylamino)(3H-[1,2,3]triazolo[4,5-b]pyridin-3-yloxy)methylidene]-N-methylmethanaminium hexafluorophosphate, the mixture was stirred at 40° C. for 5 h. The reaction mixture was filtered and the filtrate was separated by means of preparative HPLC (Method 11). 15 mg (9% of theory) of the title compound were obtained.

LC-MS (Method 5): Rₜ=0.99 min; MS (ESIpos): m/z=767 [M+H]⁺.

### Example 40A

### N-alpha-[(trans-4-{[(tert-Butoxycarbonyl)amino]methyl}cyclohexyl)carbonyl]-4-(6-{[(2R)-1-hydroxypropan-2-yl]carbamoyl}-2-methylpyridin-3-yl)-N-[4-(2H-tetrazol-5-yl)phenyl]-L-phenylalaninamide trifluoroacetate

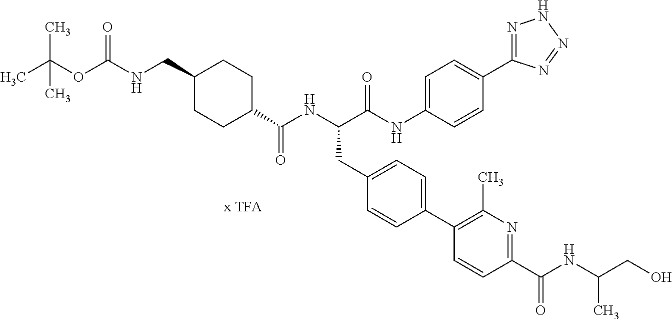

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 22.0 mg (0.29 mmol) of DL-alaninol in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate and stirred at RT for 24 h. The reaction mixture was filtered and the filtrate was separated by means of preparative HPLC (eluent: acetonitrile/water with 0.1% TFA (gradient)). 65 mg (46% of theory) of the title compound were obtained.

LC-MS (Method 1): Rₜ=0.95 min; MS (ESIpos): m/z=740.4 [M+H-TFA]⁺.

### Example 41A

### N-alpha-[(trans-4-{[(tert-Butoxycarbonyl)amino]methyl}cyclohexyl)carbonyl]-4-{2-methyl-6-[(1-methylpiperidin-4-yl)carbamoyl]pyridin-3-yl}-N-[4-(2H-tetrazol-5-yl)phenyl]-L-phenylalaninamide trifluoroacetate

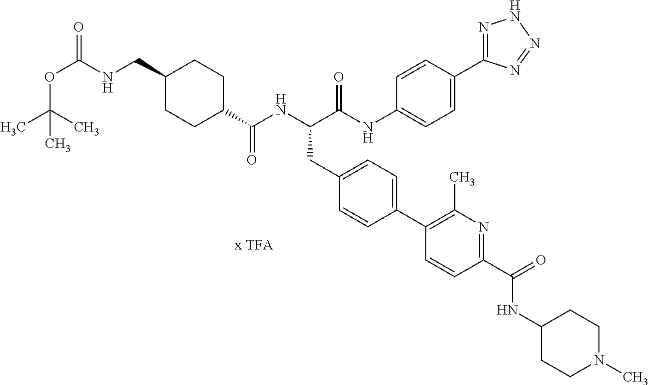

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 33.4 mg (0.29 mmol) of 1-methylpiperidin-4-amine in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 16 h. The reaction mixture was separated by means of preparative HPLC (eluent: acetonitrile/water with 0.01% TFA (gradient)). The product-containing fractions were combined and concentrated on a rotary evaporator. The residue was dried under high vacuum. 92 mg (54% of theory, 76% purity) of the title compound were obtained.

LC-MS (Method 1): Rₜ=0.82 min; MS (ESIpos): m/z=779 [M+H-TFA]⁺.

### Example 42A

### 4-{6-[(trans-4-Aminocyclohexyl)carbamoyl]-2-methylpyridin-3-yl}1-N-alpha-[(trans-4-{[(tert-butoxycarbonyl)amino]methyl}cyclohexyl)carbonyl]-N-[4-(2H-tetrazol-5-yl)phenyl]-L-phenylalaninamide trifluoroacetate

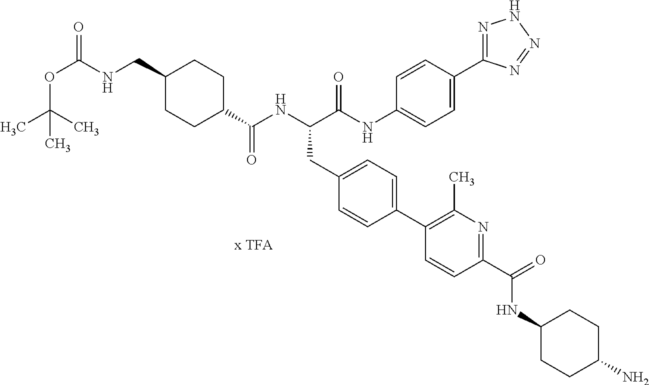

A solution of 100 mg (0.15 mmol) of 5-{4-[(2S)-2-{[(trans-4-{[(tert-butoxycarbonyl)amino]-methyl}cyclohexyl)carbonyl]amino}-3-oxo-3-{[4-(2H-tetrazol-5-yl)phenyl]amino}propyl]phenyl}-6-methylpyridine-2-carboxylic acid and 100.4 mg (0.88 mmol) of trans-cyclohexane-1,4-diamine in 1.25 ml of dimethylformamide was admixed with 77 μl (0.44 mmol) of N,N-diisopropylethylamine and 83.5 mg (0.22 mmol) of N-[(dimethylamino)(3H-[1,2,3]triazolo[4,5-b]pyridin-3-yloxy)methylidene]-N-methylmethanaminium hexafluorophosphate. The mixture was stirred at RT for 16 h. The reaction mixture was separated by means of preparative HPLC (eluent: acetonitrile/water with 0.01% TFA (gradient)). The product-containing fractions were combined and concentrated on a rotary evaporator. The residue was dried under high vacuum. 71 mg (46% of theory, 83% purity) of the title compound were obtained.

LC-MS (Method 1): Rₜ=0.82 min; MS (ESIpos): m/z=779.4 [M+H-TFA]⁺.

### Example 43A

### N-alpha-[(trans-4-{[(tert-Butoxycarbonyl)amino]methyl}cyclohexyl)carbonyl]-4-{4-methyl-6-[(3-oxopiperazin-1-yl)carbonyl]pyridin-3-yl}-N-[4-(1H-tetrazol-5-yl)phenyl]-L-phenylalaninamide

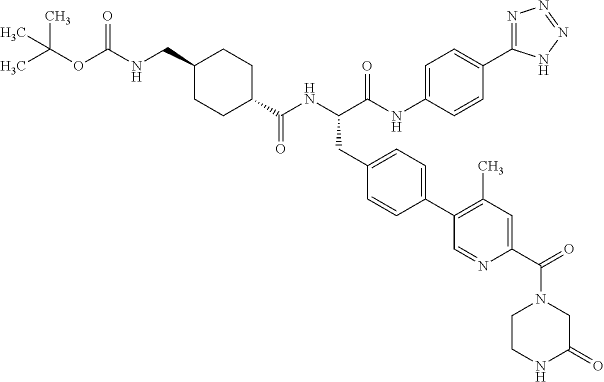 [SMILES: Cc1cc(C(=O)N2CCNC(=O)C2)ncc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 44.0 mg (0.44 mmol) of piperazin-2-one in 2 ml of dimethyl sulphoxide was admixed with 115 μl (0.66 mmol) of N,N-diisopropylethylamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for 4 h. The reaction mixture was filtered and the filtrate was separated by means of preparative HPLC (Method 11). 34 mg (20% of theory) of the title compound were obtained.

LC-MS (Method 5): Rₜ=0.73 min; MS (ESIpos): m/z=765.4 [M+H]⁺.

### Example 44A

### Ethyl 5-{4-[(2S)-2-{[(trans-4-{[(tert-butoxy carbonyl)amino]methyl}cyclohexyl)carbonyl]amino}-3-oxo-3-{[4-(2H-tetrazol-5-yl)phenyl]amino}propyl]phenyl}-6-methylpyridine-2-carboxylate trifluoroacetate

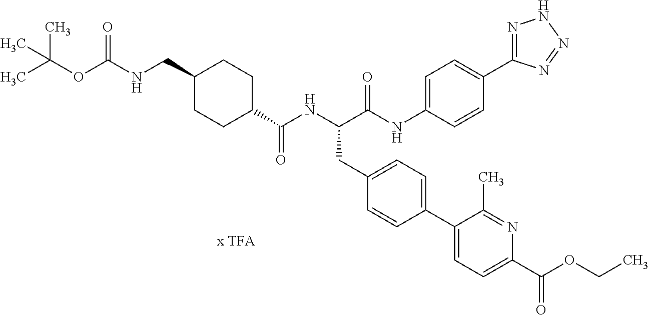

A solution of 102.8 mg (0.45 mmol) of methyl 5-bromo-6-methylpyridine-2-carboxylate and 121.6 mg (0.48 mmol) of bis(pinacolato)diborane in 2.5 ml of toluene was admixed with 93.9 mg (0.96 mmol) of potassium acetate and degassed with argon for 5 min 13.0 mg (0.02 mmol) of [1,1-bis-(diphenylphosphine)ferrocene]dichloropalladium-dichloromethane complex were added and the mixture was stirred at 120° C. in a preheated oil bath for 3 h. The mixture was concentrated and the residue was taken up in 2.5 ml of 1,2-dimethoxyethane and 1 ml of ethanol. 200 mg (0.32 mmol) of 4-bromo-N-alpha-[(trans-4-{[(tert-butoxycarbonyl)amino]methyl}cyclohexyl)-carbonyl]-N-[4-(2H-tetrazol-5-yl)phenyl]-L-phenylalaninamide, 13.0 mg (0.02 mmol) of [1,1-bis(diphenylphosphine)ferrocene]dichloropalladium-dichloromethane complex and 0.36 ml (0.72 mmol) of 2M sodium carbonate solution in water were added, and the mixture was stirred under reflux overnight. The reaction mixture was admixed with a little dimethylformamide, water and acetonitrile, filtered and separated twice by means of preparative HPLC (eluent: acetonitrile/water with 0.1% TFA (gradient)). The product-containing test tubes were concentrated and dried under high vacuum. The residue was recrystallized from a little methanol, filtered off with suction and dried again under high vacuum. 33 mg (14% of theory) of the title compound were obtained.

LC-MS (Method 1): Rₜ=1.04 min; MS (ESIpos): m/z=711 [M+H-TFA]⁺.

### Example 45A

### N-alpha-[(trans-4-{[(tert-Butoxycarbonyl)amino]methyl}cyclohexyl)carbonyl]-4-{4-methyl-6-[(4-methylpiperazin-1-yl)carbonyl]pyridin-3-yl}-N-[4-(1H-tetrazol-5-yl)phenyl]-L-phenylalaninamide

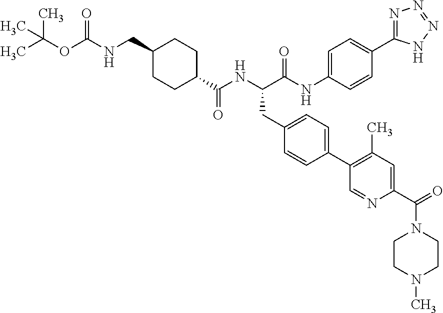 [SMILES: Cc1cc(C(=O)N2CCN(C)CC2)ncc1-c1ccc(C[C@H](NC(=O)[C@H]2CC[C@H](CNC(=O)OC(C)(C)C)CC2)C(=O)Nc2ccc(-c3nnn[nH]3)cc2)cc1]

A solution of 150 mg (0.22 mmol) of 5-{4-[(2S)-2-{[(trans-4-{[(tert-butoxycarbonyl)amino]-methyl}cyclohexyl)carbonyl]amino}-3-oxo-3-{[4-(1H-tetrazol-5-yl)phenyl]amino}propyl]phenyl}-4-methylpyridine-2-carboxylic acid and 44.0 mg (0.44 mmol) of 1-methylpiperazine in 2 ml of dimethyl sulphoxide was admixed with 115 μl (0.66 mmol) of N,N-diisopropylethylamine and 167.1 mg (0.44 mmol) of N-[(dimethylamino)(3H-[1,2,3]triazolo[4,5-b]pyridin-3-yloxy)methylidene]-N-methylmethanaminium hexafluorophosphate and stirred at RT for 24 h and at 40° C. for 4 h. The reaction mixture was filtered and separated by means of preparative HPLC (Method 11). 25 mg (15% of theory) of the title compound were obtained.

LC-MS (Method 5): Rₜ=0.77 min; MS (ESIpos): m/z=765.4 [M+H]⁺.

### Example 46A

### N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenylalaninamide

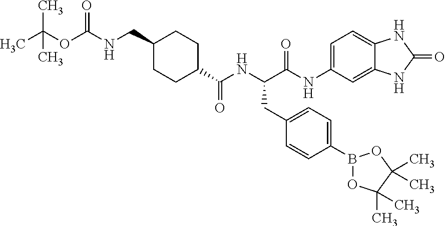

4-Bromo-N-alpha-[(trans-4-{[(tert-butoxycarbonyl)amino]methyl}cyclohexyl)carbonyl]-N-(2-oxo-2,3-dihydro-1H-benzimidazol-5-yl)-L-phenylalaninamide (5.0 g, 8.14 mmol) and 4,4,4′,4′,5,5,5′,5′-octamethyl-2,2′-bi-1,3,2-dioxaborolane (3.1 g, 12.2 mmol) were dissolved in 60 ml of DMSO, 1,1′-bis(diphenylphosphino)ferrocene-dichloropalladium(II) (332 mg, 0.4 mmol) and potassium acetate (2.4 g, 24.4 mmol) were added and the mixture was stirred at 110° C. for 4 h and then converted further as crude product.

LC-MS (Method 4): Rₜ=1.27 min; MS (ESIpos): m/z=662.5 [M+H]⁺.

### Example 47A

### 5-Bromo-N-cyclobutyl-6-methylpyridine-2-carboxamide

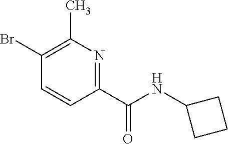

A solution of 1.0 g (4.6 mmol) of 5-bromo-6-methylpyridine-2-carboxylic acid and 553 mg (5.0 mmol) of cyclobutylamine in 60 ml of ethyl acetate was admixed with 2.5 ml (18.1 mmol) N,N-diisopropylethylamine and 7.2 g (50% in ethyl acetate, 11.3 mmol) of 2,4,6-tripropyl-1,3,5,2,4,6-trioxatriphosphinane 2,4,6-trioxide solution and refluxed for 3 h. The reaction mixture was admixed with water, the phases were separated and the aqueous phase was extracted twice with ethyl acetate. The combined organic phases were washed twice with saturated aqueous ammonium chloride solution and once with saturated aqueous sodium chloride solution, dried over sodium sulphate, filtered and concentrated under reduced pressure. This gave 1.24 g (quant.) of the title compound. This was converted further as the crude product.

LC-MS (Method 4): Rₜ=1.23 min; MS (ESIpos): m/z=271.0 [M+H]⁺.

### Example 48A

### 5-Bromo-6-methyl-N-(1,1,1-trifluoropropan-2-yl)pyridine-2-carboxamide

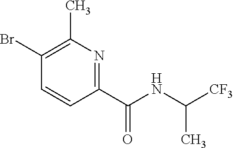 [SMILES: Cc1nc(C(=O)NC(C)C(F)(F)F)ccc1Br]

A solution of 1.0 g (4.6 mmol) of 5-bromo-6-methylpyridine-2-carboxylic acid and 761 mg (5.1 mmol) of 1,1,1-trifluoropropan-2-amine in 61 ml of ethyl acetate was admixed with 2.0 ml (13.9 mmol) N,N-diisopropylethylamine and 8.8 g (50% in ethyl acetate, 8.2 ml, 13.9 mmol) of 2,4,6-tripropyl-1,3,5,2,4,6-trioxatriphosphinane 2,4,6-trioxide solution, refluxed for 1 h and stirred at RT for 48 h. The reaction mixture was admixed with water, the phases were separated and the aqueous phase was extracted three times with ethyl acetate. The combined organic phases were washed once with saturated aqueous sodium chloride solution, dried over sodium sulphate, filtered and concentrated under reduced pressure. The residue was purified by chromatography via Biotage (column: SNAP, flow rate 25 ml/min, n-hexane/ethyl acetate gradient). This gave 1.42 g (99% of theory) of the title compound.

LC-MS (Method 4): Rt=1.30 min; MS (ESIpos): m/z=313.0 [M+H]⁺.

### Example 49A

### 4-Bromo-N-alpha-[(trans-4-{[(tert-butoxycarbonyl)amino]methyl}cyclohexyl)carbonyl]-N-1H-indazol-6-yl-L-phenylalaninamide

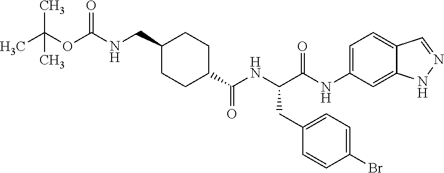

A solution of 4-bromo-N-[(trans-4-{[(tert-butoxy carbonyl)-amino]methyl}-cyclohexyl)-carbonyl]-L-phenylalanine (2000 mg, 4 mmol) and 6-aminoindazole (606 mg, 5 mmol) in dimethylformamide (30 ml) was admixed with N,N-diisopropylethylamine (1.8 ml, 10 mmol). The suspension was admixed with a 2,4,6-tripropyl-1,3,5,2,4,6-trioxatriphosphinane 2,4,6-trioxide solution (50% in dimethylformamide, 3.2 mg, 5 mmol) and with dimethylformamide until dissolution, and then the mixture was stirred at RT for 16 h. The reaction mixture was stirred into ethyl acetate (2500 ml), and washed three times with water (300 ml) and once with saturated aqueous sodium chloride solution. The organic phase was dried with sodium sulphate and the solvent was removed. The crude product was stirred with acetonitrile and filtered off with suction. This gave 1400 mg (54% of theory) of the title compound.

¹H NMR (400 MHz, DMSO-d₆): δ=0.68-0.98 (m, 2H), 1.05-1.31 (m, 4H), 1.39 (s, 9H), 1.46-1.76 (m, 4H), 1.98-2.15 (m, 1H), 2.65-3.07 (m, 4H), 4.56-4.71 (m, 1H), 6.71-6.83 (m, 1H), 7.25 (d, 2H), 7.47 (d, 2H), 7.72-7.84 (m, 4H), 8.10-8.20 (m, 1H), 10.45 (s, 1H), 12.86 (br. s, 1H).

LC-MS (Method 1): Rₜ=1.09 min; MS (ESIpos): m/z=598 [M+H]⁺.

### Example 50A

### N-alpha-[(trans-4-{[(tert-Butoxycarbonyl)amino]methyl}cyclohexyl)carbonyl]-N-1H-indazol-6-yl-4-(4,4,5,5-tetramethyl-1,3,2-dioxaborolan-2-yl)-L-phenylalaninamide

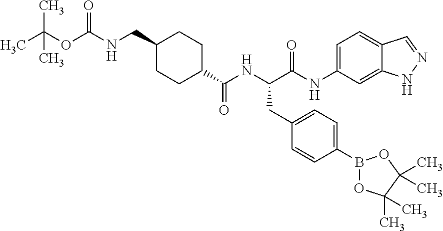

4-Bromo-N-alpha-[(trans-4-{[(tert-butoxycarbonyl)amino]methyl}cyclohexyl)carbonyl]-N-1H-indazol-6-yl-L-phenylalaninamide (4.0 g, 6.7 mmol) and 4,4,4′,4′,5,5,5′,5′-octamethyl-2,2′-bi-1,3,2-dioxaborolane (2.55 g, 10.0 mmol) were dissolved in 40 ml of DMSO, 1,1′-bis(diphenylphosphino)ferrocene-dichloropalladium(II) (273 mg, 0.33 mmol) and potassium acetate (1.97 g, 20.0 mmol) were added and the mixture was stirred at 120° C. for 4 h and then converted further as crude product.

LC-MS (Method 4): Rₜ=1.37 min; MS (ESIpos): m/z=646.5 [M+H]⁺.

### Example 51A

### N-alpha-[(trans-4-{[(tert-Butoxycarbonyl)amino]methyl}cyclohexyl)carbonyl]-4-[6-(cyclobutylcarbamoyl)-2-methylpyridin-3-yl]-N-1H-indazol-6-yl-L-phenylalaninamide

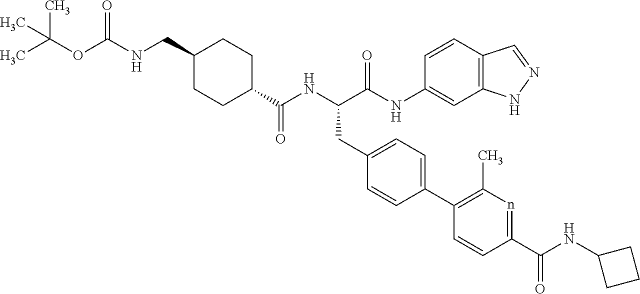

N-alpha-[(trans-4-{[(tert-Butoxycarbonyl)amino]methyl}cyclohexyl)carbonyl]-N-1H-indazol-6-yl-4-(4,4,5,5-tetramethyl-1,3,2-dioxaborolan-2-yl)-L-phenylalaninamide (250 mg, 50%, 0.19 mmol) and 5-bromo-N-cyclobutyl-6-methylpyridine-2-carboxamide (57.3 mg, 0.21 mmol) were dissolved in dimethyl sulphoxide (2.5 ml) and admixed with 1,1′-bis(diphenylphosphino)ferrocene-dichloropalladium(II) (16 mg, 19 μmol), sodium carbonate (61.6 mg, 0.6 mmol) and water (0.29 ml, 16 mmol). The reaction mixture was stirred at 110° C. for 2 h, filtered through deactivated alumina and purified via HPLC (Method 10). This gave 76 mg (55% of theory) of the title compound.

LC-MS (Method 4): 1.29 min; MS (ESIpos): m/z=708.5 [M+H]⁺.

### Example 52A

### N-alpha-[(trans-4-{[(tert-Butoxycarbonyl)amino]methyl}cyclohexyl)carbonyl]-N-1H-indazol-6-yl-4-{2-methyl-6-[(1,1,1-trifluoropropan-2-yl)carbamoyl]pyridin-3-yl}-L-phenylalaninamide

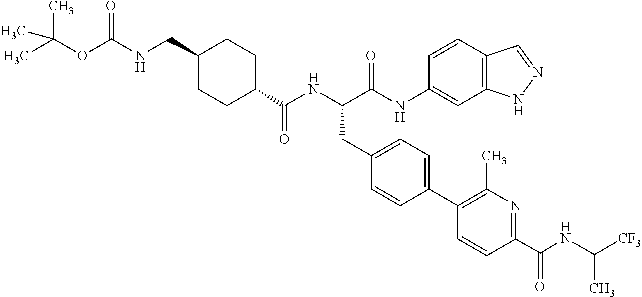 [SMILES: Cc1nc(C(=O)NC(C)C(F)(F)F)ccc1-c1ccc(C[C@H](NC(=O)[C@H]2CC[C@H](CNC(=O)OC(C)(C)C)CC2)C(=O)Nc2ccc3cn[nH]c3c2)cc1]

N-alpha-[(trans-4-{[(tert-Butoxycarbonyl)amino]methyl}cyclohexyl)carbonyl]-N-1H-indazol-6-yl-4-(4,4,5,5-tetramethyl-1,3,2-dioxaborolan-2-yl)-L-phenylalaninamide (250 mg, 50%, 0.19 mmol) and 5-bromo-6-methyl-N-(1,1,1-trifluoropropan-2-yl)pyridine-2-carboxamide (66.3 mg, 0.21 mmol) were dissolved in dimethyl sulphoxide (2.5 ml) and admixed with 1,1′-bis(diphenylphosphino)ferrocene-dichloropalladium(II) (16 mg, 19 μmol), sodium carbonate (61.6 mg, 0.6 mmol) and water (0.29 ml, 16 mmol). The reaction mixture was stirred at 110° C. for 2 h, filtered through deactivated alumina and purified via HPLC (Method 11). This gave 88 mg (61% of theory) of the title compound.

LC-MS (Method 4): 1.33 min; MS (ESIpos): m/z=750.5 [M+H]⁺.

### Example 53A

### N-alpha-[(trans-4-{[(tert-Butoxycarbonyl)amino]methyl}cyclohexyl)carbonyl]-4-{2-methyl-6-[(1,1,1-trifluoropropan-2-yl)carbamoyl]pyridin-3-yl}-N-(2-oxo-2,3-dihydro-1H-benzimidazol-5-yl)-L-phenylalaninamide

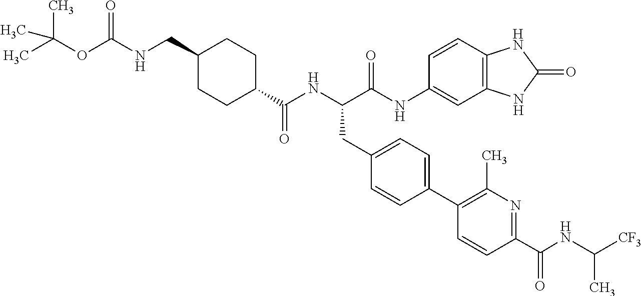 [SMILES: Cc1nc(C(=O)NC(C)C(F)(F)F)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenylalaninamide (170 mg, 0.26 mmol) and 5-bromo-6-methyl-N-(1,1,1-trifluoropropan-2-yl)pyridine-2-carboxamide (87.9 mg, 0.28 mmol) were dissolved in dimethyl sulphoxide (2 ml) and admixed with tetrakis(triphenylphosphine)palladium(0) (29.7 mg, 26 μmol), sodium carbonate (81.7 mg, 0.8 mmol) and water (0.39 ml, 21.5 mmol). The reaction mixture was stirred at 100° C. for 2 h, admixed with 1,1′-bis(diphenylphosphino)ferrocene-dichloropalladium(II) (21 mg, 26 μmol) and stirred at 120° C. for a further 2 h. This was followed by filtration through deactivated alumina and purification via HPLC (Method 11). This gave 58 mg (29% of theory) of the title compound.

LC-MS (Method 4): 1.25 min; MS (ESIpos): m/z=766.5 [M+H]⁺.

### Example 54A

### N-alpha-[(trans-4-{[(tert-Butoxycarbonyl)amino]methyl}cyclohexyl)carbonyl]-4-[6-(cyclobutylcarbamoyl)-2-methylpyridin-3-yl]-N-(2-oxo-2,3-dihydro-1H-benzimidazol-5-yl)-L-phenylalaninamide

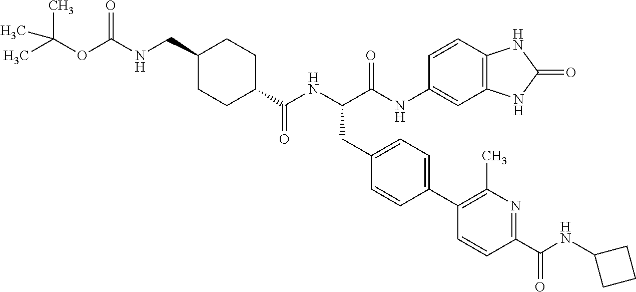 [SMILES: Cc1nc(C(=O)NC2CCC2)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

N-alpha-[(trans-4-{[(tert-Butoxycarbonyl)amino]methyl}cyclohexyl)carbonyl]-N-(2-oxo-2,3-dihydro-1H-benzimidazol-5-yl)-4-(4,4,5,5-tetramethyl-1,3,2-dioxaborolan-2-yl)-L-phenylalaninamide (170 mg, 0.26 mmol) and 5-bromo-N-cyclobutyl-6-methylpyridine-2-carboxamide (76.1 mg, 0.28 mmol) were dissolved in dimethyl sulphoxide (2 ml) and admixed with tetrakis(triphenylphosphine)palladium(0) (29.7 mg, 26 μmol), sodium carbonate (81.7 mg, 0.8 mmol) and water (0.39 ml, 21.5 mmol). The reaction mixture was stirred at 100° C. for 2 h, admixed with 1,1′-bis(diphenylphosphino)ferrocene-dichloropalladium(II) (21 mg, 26 μmol) and stirred at 120° C. for a further 2 h. This was followed by filtration through deactivated alumina and purification via HPLC (Method 10). This gave 83 mg (44% of theory) of the title compound.

LC-MS (Method 4): 1.20 min; MS (ESIpos): m/z=724.5 [M+H]⁺.

### Example 55A

### 5-Bromo-N-isopropyl-6-methylpyridine-2-carboxamide

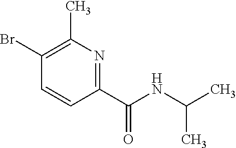

A solution of 5-bromo-6-methylpyridine-2-carboxylic acid (1.07 g, 4.94 mmol) and isopropylamine (0.93 ml, 10.88 mmol) in THF (15 ml) was admixed with N,N-diisopropylethylamine (1.72 ml, 9.89 mmol), and HATU (2.82 g, 7.42 mmol) was added. The reaction solution was stirred at RT for 3 days, then additional N,N-diisopropylamine (0.84 ml, 9.89 mmol) was added and the solution was stirred at 60° C. for 3 h. The precipitated solid was filtered off, then the filtrate was diluted with ethyl acetate and washed with water and saturated aqueous sodium chloride solution. The organic phase was dried over sodium sulphate and filtered, and the solvent was removed on a rotary evaporator. This gave 1.19 g (93% of theory) of the title compound.

LC-MS (Method 2): Rₜ=2.12 min; MS (ESIpos): m/z=257 [M+H]⁺.

### Example 56A

### [6-(Isopropylcarbamoyl)-2-methylpyridin-3-yl]boric acid

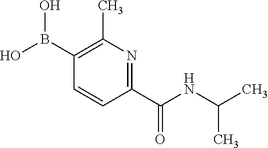 [SMILES: Cc1nc(C(=O)NC(C)C)ccc1B(O)O]

A solution of 5-bromo-N-isopropyl-6-methylpyridine-2-carboxamide (1.0 g, 3.89 mmol), bis(pinacolato)diborane (1.09 g, 4.28 mmol) and potassium acetate (0.76 g, 7.78 mmol) in toluene (20 ml) was degassed with argon and then admixed with [1,1-bis(diphenylphosphino)ferrocene]-dichloropalladium-dichloromethane complex (159 mg, 0.19 mmol). The mixture was then stirred at 110° C. for 5 h. The reaction mixture was concentrated on a rotary evaporator and dried under high vacuum. The residue (864 mg, 100% of theory) was used further without purification.

LC-MS (Method 2): Rₜ=1.43 min; MS (ESIpos): m/z=223 [M+H]⁺.

### Example 57A

### N-[(trans-4-{[(tert-butoxycarbonyl)amino]methyl}cyclohexyl)carbonyl]-4-[6-(isopropylcarbamoyl)-2-methylpyridin-3-yl]-L-phenylalanine

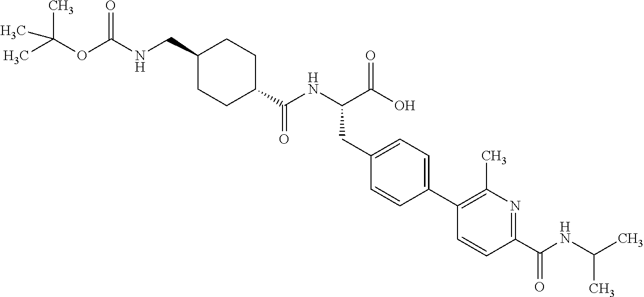 [SMILES: Cc1nc(C(=O)NC(C)C)ccc1-c1ccc(C[C@H](NC(=O)[C@H]2CC[C@H](CNC(=O)OC(C)(C)C)CC2)C(=O)O)cc1]

A solution of 4-bromo-N-[(trans-4-{[(tert-butoxycarbonyl)amino]methyl}cyclohexyl)-carbonyl]-L-phenylalanine (900 mg, 1.86 mmol) and [6-(isopropylcarbamoyl)-2-methylpyridin-3-yl]boric acid (868 mg, 3.91 mmol) in 1,2-dimethoxyethane (15 ml) and ethanol (6 ml) was degassed with argon and admixed with 2N aqueous sodium carbonate solution (1.86 ml, 3.72 mmol) and [1,1-bis-(diphenylphosphino)ferrocene]dichloropalladium-dichloromethane complex (255.4 mg, 0.31 mmol). The mixture was then stirred at reflux (oil bath temperature 100° C.) for 5 h. The reaction mixture was concentrated on a rotary evaporator and the residue was dissolved in a little DMSO. The solution was filtered through a Millipore filter and purified by preparative HPLC (eluent: gradient of acetonitrile/water with 0.1% trifluoroacetic acid). This gave 857 mg (67% of theory) of the title compound.

¹H NMR (400 MHz, DMSO-d₆): δ=ppm 0.71-0.90 (m, 2H), 1.05-1.30 (m, 9H), 1.47-1.55 (m, 1H), 1.64 (m, 3H), 1.97-2.12 (m, 1H), 2.54 (s, 3H), 2.74 (m, 2H), 2.91 (m, 1H), 3.14 (m, 1H), 4.06-4.22 (m, 1H), 4.42-4.58 (m, 1H), 6.76 (m, 1H), 7.34 (s, 4H), 7.74 (d, 1H), 7.90 (d, 1H), 8.03 (d, 1H), 8.29 (d, 1H), 12.67 (br. s, 1H).

LC-MS (Method 1): Rₜ=1.01 min; MS (ESIneg): m/z=579 [M−H]⁻.

### Example 58A

### N-alpha-[(trans-4-{[(tert-Butoxycarbonyl)amino]methyl}cyclohexyl)carbonyl]-N-(7-chloro-2-oxo-2,3-dihydro-1,3-benzoxazol-5-yl)-4-[6-(isopropylcarbamoyl)-2-methylpyridin-3-yl]-L-phenylalaninamide

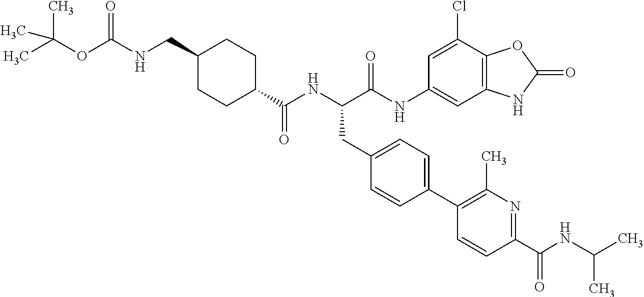 [SMILES: Cc1nc(C(=O)NC(C)C)ccc1-c1ccc(C[C@H](NC(=O)[C@H]2CC[C@H](CNC(=O)OC(C)(C)C)CC2)C(=O)Nc2cc(Cl)c3oc(=O)[nH]c3c2)cc1]

A suspension of N-[(trans-4-{[(tert-butoxycarbonyl)amino]methyl}cyclohexyl)carbonyl]-4-[6-(isopropylcarbamoyl)-2-methylpyridin-3-yl]-L-phenylalanine (100 mg, 0.17 mmol) in ethyl acetate (2.5 ml) was admixed with 5-amino-7-chloro-1,3-benzoxazol-2(3H)-one (35 mg, 0.19 mmol) and N,N-diisopropylethylamine (0.09 ml, 0.52 mmol). The suspension was admixed with a 2,4,6-tripropyl-1,3,5,2,4,6-trioxatriphosphinane 2,4,6-trioxide solution (50% in DMF, 0.30 ml, 0.52 mmol) and then the mixture was stirred at reflux (oil bath temperature 80° C.) for 3 h. The reaction mixture was admixed with DMSO (1 ml) and the ethyl acetate was removed on a rotary evaporator. The residue was filtered through a Millipore filter and purified by preparative HPLC (eluent: gradient of acetonitrile/water with 0.1% trifluoroacetic acid). This gave 44 mg (34% of theory) of the title compound.

LC-MS (Method 13): Rₜ=3.75 min; MS (ESIneg): m/z=745 [M−H]⁻.

### Example 59A

### N-alpha-[(trans-4-{[(tert-Butoxycarbonyl)amino]methyl}cyclohexyl)carbonyl]-4-[6-(isopropylcarbamoyl)-2-methylpyridin-3-yl]-N-(3-oxo-2,3-dihydro-1H-indazol-6-yl)-L-phenylalaninamide

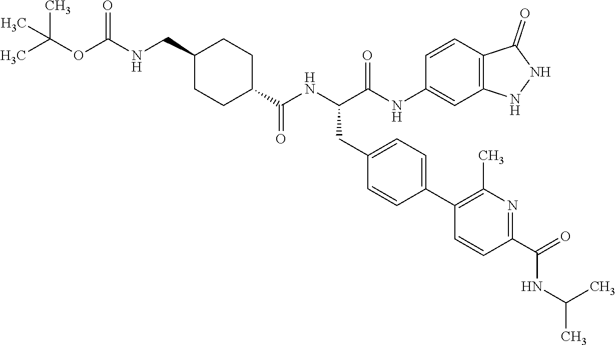 [SMILES: Cc1nc(C(=O)NC(C)C)ccc1-c1ccc(C[C@H](NC(=O)[C@H]2CC[C@H](CNC(=O)OC(C)(C)C)CC2)C(=O)Nc2ccc3c(=O)[nH][nH]c3c2)cc1]

A solution of 4-bromo-N-alpha-[(trans-4-{[(tert-butoxycarbonyl)amino]methyl}cyclohexyl)-carbonyl]-N-(3-oxo-2,3-dihydro-1H-indazol-6-yl)-L-phenylalaninamide (200 mg, 0.33 mmol) and [6-(isopropylcarbamoyl)-2-methylpyridin-3-yl]boric acid (152 mg, 0,683 mmol) in 1,2-dimethoxyethane (3 ml) and ethanol (1.2 ml) was degassed with argon and admixed with 2N aqueous sodium carbonate solution (0.33 ml, 0.65 mmol) and [1,1-bis-(diphenylphosphino)ferrocene]dichloropalladium-dichloromethane complex (13 mg, 0.016 mmol). The mixture was then stirred at reflux (oil bath temperature 100° C.) for 5 h. The reaction mixture was concentrated on a rotary evaporator and the residue was dissolved in DMSO (1 ml). The solution was filtered through a Millipore filter and purified by preparative HPLC (eluent: gradient of acetonitrile/water with 0.1% trifluoroacetic acid). This gave 77 mg (29% of theory) of the title compound.

LC-MS (Method 1): Rₜ=0.97 min; MS (ESIneg): m/z=710 [M−H]⁻.

### Example 60A

### Methyl 6-methyl-5-(4,4,5,5-tetramethyl-1,3,2-dioxaborolan-2-yl)pyridine-2-carboxylate

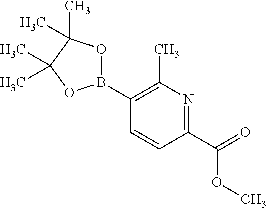

A solution of methyl 5-bromo-6-methylpyridine-2-carboxylate (3.0 g, 13.04 mmol), bis(pinacolato)diborane (4.97 g, 19.56 mmol) and potassium acetate (3.84 g, 39.12 mmol) in toluene (45 ml) was degassed with argon and then admixed with [1,1-bis(diphenylphosphino)ferrocene]-dichloropalladium-dichloromethane complex (532.4 mg, 0.65 mmol). The mixture was then stirred at 110° C. for 4.5 h. The reaction mixture was filtered through Celite and eluted with ethyl acetate, and the filtrate was washed with saturated aqueous sodium chloride solution. The organic phase was dried over sodium sulphate, filtered and concentrated on a rotary evaporator, and the residue was dried under high vacuum. The crude product (3.6 g, 100% of theory) was used further without purification.

LC-MS (Method 12): Rₜ=1.77 min; MS (ESIpos): m/z=278 [M+H]⁺.

### Example 61A

### N-[(trans-4-{[(tert-Butoxycarbonyl)amino]methyl}cyclohexyl)carbonyl]-4-[6-(methoxycarbonyl)-2-methylpyridin-3-yl]-L-phenylalanine

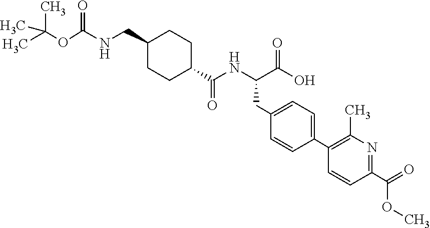 [SMILES: COC(=O)c1ccc(-c2ccc(C[C@H](NC(=O)[C@H]3CC[C@H](CNC(=O)OC(C)(C)C)CC3)C(=O)O)cc2)c(C)n1]

A solution of 4-bromo-N-[(trans-4-{[(tert-butoxycarbonyl)amino]methyl}cyclohexyl)-carbonyl]-L-phenylalanine (4.24 g, 8.78 mmol) and methyl 6-methyl-5-(4,4,5,5-tetramethyl-1,3,2-dioxaborolan-2-yl)pyridine-2-carboxylate (3.6 g, 12.29 mmol) in 1,2-dimethoxyethane (30 ml) and methanol (10 ml) was degassed with argon and then admixed with 2N aqueous sodium carbonate solution (8.78 ml, 17.55 mmol) and [1, 1-bis(diphenylphosphino)ferrocene]dichloropalladium-dichloromethane complex (716.6 mg, 0.88 mmol). The mixture was then stirred at reflux (oil bath temperature 80° C.) for 8 h. The reaction mixture was filtered through Celite and eluted with ethyl acetate, and the filtrate was concentrated on a rotary evaporator. The residue was taken up in ethyl acetate (20 ml) and 10% aqueous citric acid solution (20 ml), and the aqueous phase removed was extracted with ethyl acetate. The combined organic phases were washed with saturated aqueous sodium chloride solution, dried over sodium sulphate and filtered, and the solvent was removed on a rotary evaporator. The residue was purified by flash chromatography (eluent: dichloromethane/methanol, 20:1 to 10:1). This gave 6.96 g (97% of theory, 68% purity) of the title compound. The product was used further without further purification.

LC-MS (Method 1): Rₜ=0.92 min; MS (ESIneg): m/z=552 [M−H]⁻.

### Example 62A

### Methyl 5-(4-{(2S)-2-{[(trans-4-{[(tert-butoxycarbonyl)amino]methyl}cyclohexyl)carbonyl]-amino}-3-oxo-3-[(2-oxo-2,3-dihydro-1H-benzimidazol-5-yl)amino]propyl}phenyl)-6-methylpyridine-2-carboxylate

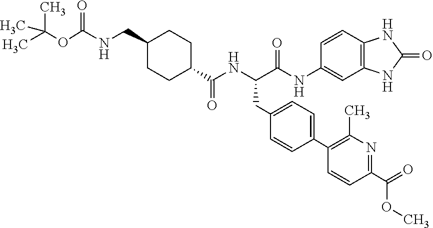 [SMILES: COC(=O)c1ccc(-c2ccc(C[C@H](NC(=O)[C@H]3CC[C@H](CNC(=O)OC(C)(C)C)CC3)C(=O)Nc3ccc4[nH]c(=O)[nH]c4c3)cc2)c(C)n1]

A solution of N-[(trans-4-{[(tert-butoxycarbonyl)amino]methyl}cyclohexyl)carbonyl]-4-[6-(methoxycarbonyl)-2-methylpyridin-3-yl]-L-phenylalanine (4.0 g, 7.23 mmol) and 5-amino-1,3-dihydro-2H-benzimidazol-2-one (2.16 g, 14.45 mmol) in DMF (40 ml) was admixed with N,N-diisopropylamine (3.78 ml, 21.67 mmol), and HATU (4.12 g, 10.84 mmol) was added thereto. The reaction mixture was stirred at RT overnight. The mixture was diluted with water (100 ml) and the precipitated solid was filtered off with suction, then washed with water and ethyl acetate and dried under high vacuum. From this, 1.75 g (28% of theory) of the title compound were obtained. The organic filtrate was concentrated on a rotary evaporator. The residue was dissolved in a little DMSO, filtered through a Millipore filter and purified by preparative HPLC (eluent: gradient of acetonitrile/water with 0.1% formic acid). This gave another 394.7 mg (7% of theory) of the title compound.

LC-MS (Method 1): Rₜ=0.91 min; MS (ESIneg): m/z=683 [M−H]⁻.

### Example 63A

### 5-(4-{(2S)-2-{[(trans-4-{[(tert-Butoxycarbonyl)amino]methyl}cyclohexyl)carbonyl]amino}-3-oxo-3-[(2-oxo-2,3-dihydro-1H-benzimidazol-5-yl)amino]propyl}phenyl)-6-methylpyridine-2-carboxylic acid

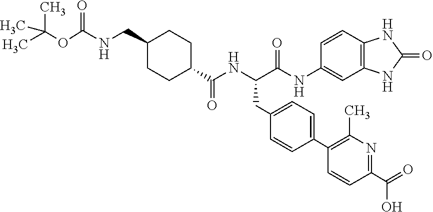 [SMILES: Cc1nc(C(=O)O)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

Methyl 5-(4-{(2S)-2-{[(trans-4-{[(tert-butoxycarbonyl)amino]methyl}cyclohexyl)carbonyl]-amino}-3-oxo-3-[(2-oxo-2,3-dihydro-1H-benzimidazol-5-yl)amino]propyl}phenyl)-6-methylpyridine-2-carboxylate (1.75 g, 2.55 mmol) was dissolved in tetrahydrofuran (23 ml), admixed with a solution of lithium hydroxide monohydrate (611 mg, 25.53 mmol) in water (7.7 ml) and stirred at RT overnight. The organic solvent was removed on a rotary evaporator and the residue was admixed with water (20 ml) and ethyl acetate (20 ml). The suspension was acidified slightly with 1N hydrochloric acid (pH 4-5). The precipitated solid was filtered off with suction, washed with water and dried under high vacuum. This gave 1.58 g of the title compound.

LC-MS (Method 1): Rₜ=0.74 min; MS (ESIneg): m/z=669 [M−H]⁻.

### Example 64A

### N-alpha-[(trans-4-{[(tert-Butoxycarbonyl)amino]methyl}cyclohexyl)carbonyl]-4-[6-(isopropylcarbamoyl)-2-methylpyridin-3-yl]-N-(2-oxo-2,3-dihydro-1H-benzimidazol-5-yl)-L-phenylalaninamide

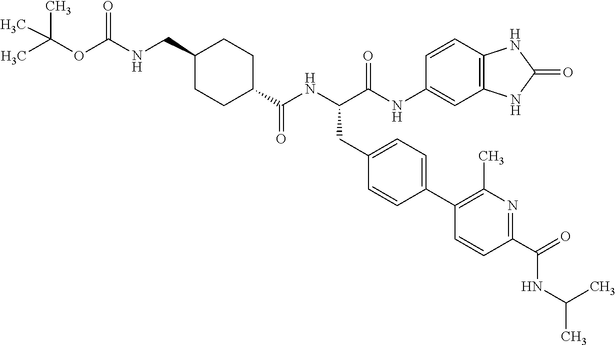 [SMILES: Cc1nc(C(=O)NC(C)C)ccc1-c1ccc(C[C@H](NC(=O)[C@H]2CC[C@H](CNC(=O)OC(C)(C)C)CC2)C(=O)Nc2ccc3[nH]c(=O)[nH]c3c2)cc1]

A solution of 5-(4-(2S)-2-{[(trans-4-{[(tert-butoxycarbonyl)amino]methyl}cyclohexyl)-carbonyl]amino}-3-oxo-3-{[(2-oxo-2,3-dihydro-1H-benzimidazol-5-yl)amino]propyl}phenyl)-6-methylpyridine-2-carboxylic acid (107 mg, 0.16 mmol) and isopropylamine (27 μl, 0.32 mmol) in DMF (2 ml) was admixed with N,N-diisopropylamine (0.11 ml, 0.64 mmol), and HATU (91 mg, 0.24 mmol) was added thereto. The reaction mixture was stirred at RT overnight (about 16 h). The residue was diluted with acetonitrile (about 2 ml) and filtered through a Millipore filter, then purified by preparative HPLC (eluent: gradient of acetonitrile/water with 0.1% formic acid). This gave 59.1 mg (50% of theory) of the title compound.

LC-MS (Method 1): Rₜ=1.00 min; MS (ESIpos): m/z=712 [M+H]⁺.

### Example 65A

### N-alpha-[(trans-4-{[(tert-Butoxycarbonyl)amino]methyl}cyclohexyl)carbonyl]-4-(4-methyl-6-{[(3R)-2-oxopiperidin-3-yl]carbamoyl}pyridin-3-yl)-N-(3-oxo-2,3-dihydro-1H-indazol-6-yl)-L-phenylalaninamide

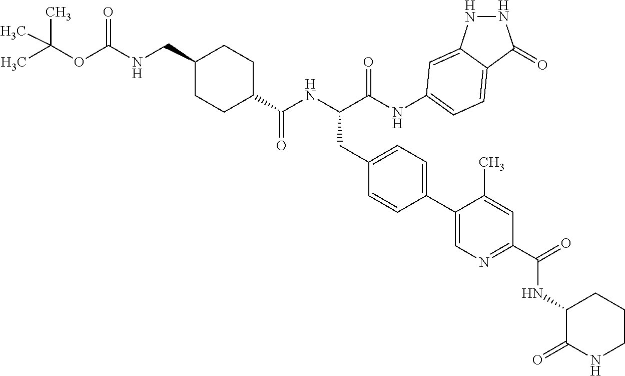

A solution of 5-(4-{(2S)-2-{[(trans-4-{[(tert-butoxycarbonyl)amino]-methyl}cyclohexyl)-carbonyl]amino}-3-oxo-3-[(3-oxo-2,3-dihydro-1H-indazol-6-yl)amino]-propyl}phenyl)-4-methylpyridine-2-carboxylic acid and (3R)-3-aminopiperidin-2-one in dimethyl sulphoxide is admixed with N,N-diisopropylethylamine and N-[(dimethylamino)(3H-[1,2,3]triazolo[4,5-b]pyridin-3-yloxy)-methylidene]-N-methyl-methanaminium hexafluorophosphate and stirred at RT overnight. After addition of 1 eq. of N-[(dimethylamino)(3H-[1,2,3]triazolo[4,5-b]pyridin-3-yloxy)methylidene]-N-methylmethanaminium hexafluorophosphate, the mixture is stirred at 40° C. for 5 h. The reaction mixture is filtered and the filtrate is separated by means of preparative HPLC. The title compound is obtained.

### Working Examples

### Example 1

### N-alpha-{[trans-4-(Aminomethyl)cyclohexyl]carbonyl}-4-[6-{[2-(diethylamino)ethyl]carbamoyl}-2-(trifluoromethyl)pyridin-3-yl]-N-[4-(2H-tetrazol-5-yl)phenyl]-L-phenylalaninamide hydrochloride

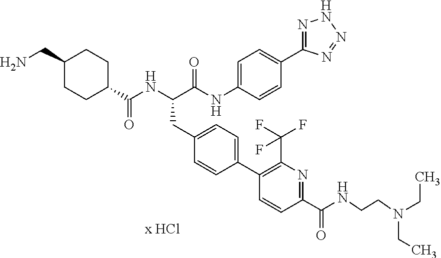 [SMILES: CCN(CC)CCNC(=O)c1ccc(-c2ccc(C[C@H](NC(=O)[C@H]3CC[C@H](CN)CC3)C(=O)Nc3ccc(-c4nn[nH]n4)cc3)cc2)c(C(F)(F)F)n1]

To a solution of 43 mg (0.046 mmol) of N-alpha-[(trans-4-{[(tert-butoxycarbonyl)amino]-methyl}cyclohexyl)carbonyl]-4-[6-{[2-(diethylamino)ethyl]carbamoyl}-2-(trifluoromethyl)pyridin-3-yl]-N-[4-(2H-tetrazol-5-yl)phenyl]-L-phenylalaninamide trifluoroacetate in 1.5 ml of dioxane was added 0.17 ml (0.68 mmol) of 4M hydrogen chloride in dioxane. The mixture was stirred at RT for 24 h. The precipitated product was filtered off with suction, washed with acetonitrile and dried under high vacuum. 34 mg (92% of theory) of the title compound were obtained.

¹H NMR (400 MHz, DMSO-d₆): δ=ppm 0.84-0.99 (m, 2H), 1.24 (t, 9H), 1.42-1.52 (m, 1H), 1.55-1.62 (m, 1H), 1.68-1.82 (m, 4H), 2.10-2.19 (m, 1H), 2.60-2.69 (m, 2H), 2.96 (dd, 1H), 3.11-3.30 (m, 8H), 3.64-3.72 (m, 2H), 4.72-4.80 (m, 1H), 7.25 (d, 2H), 7.41 (d, 2H), 7.49 (d, 1H), 7.68-7.80 (m, 3H), 7.83 (d, 2H), 8.01 (d, 2H), 8.22 (d, 1H), 8.27-8.33 (m, 2H), 9.12-9.24 (m, 1H), 9.76-9.94 (m, 1H), 10.56 (br. s, 1H).

LC-MS (Method 1): Rₜ=0.61 min; MS (ESIpos): m/z=735.4 [M+H—HCl]⁺.

### Example 2

### N-alpha-{[trans-4-(Aminomethyl)cyclohexyl]carbonyl}-4-(6-{[4-(dimethylamino)cyclohexyl]-carbamoyl}-2-methylpyridin-3-yl)-N-[4-(2H-tetrazol-5-yl)phenyl]-L-phenylalaninamide hydrochloride

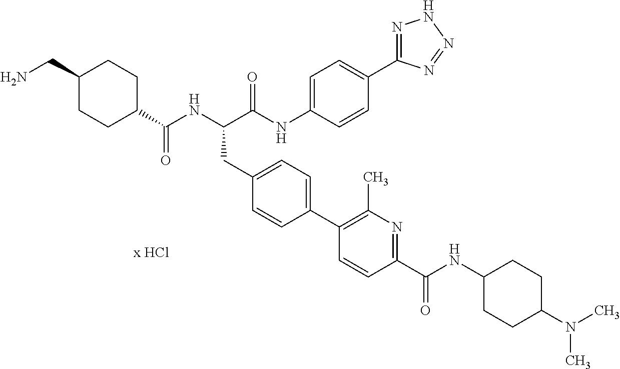

To a solution of 87.7 mg (0.1 mmol) of N-alpha-[(trans-4-{[(tert-butoxycarbonyl)amino]-methyl}cyclohexyl)carbonyl]-4-(6-{[4-(dimethylamino)cyclohexyl]carbamoyl}-2-methylpyridin-3-yl)-N-[4-(2H-tetrazol-5-yl)phenyl]-L-phenylalaninamide trifluoroacetate in 3 ml of dioxane was added 0.36 ml (1.4 mmol) of 4M hydrogen chloride in dioxane. The mixture was stirred at RT for 72 h. The precipitated product was filtered off with suction, washed with dioxane and dried under high vacuum. 38 mg (52% of theory) of the title compound were obtained.

¹H NMR (400 MHz, DMSO-d₆): δ=ppm 0.83-1.00 (m, 2H), 1.10-1.35 (m, 2H), 1.41-1.82 (m, 9H), 1.96 (m, 3H), 2.14 (m, 1H), 2.45 (s, 2H), 2.54 (s, 3H), 2.59-2.67 (m, 2H), 2.71 (m, 3H), 2.75 (d, 2H), 3.00 (m, 1H), 3.15 (m, 2H), 3.77-3.90 (m, 1H), 4.76 (m, 1H), 7.33-7.37 (m, 2H), 7.41-7.48 (m, 2H), 7.75 (t, 1H), 7.80-7.94 (m, 6H), 8.03 (d, 2H), 8.32 (d, 1H), 8.44 (d, 1H), 10.27-10.46 (m, 1H), 10.37-10.46 (m, 1H), 10.58 (m, 1H)

LC-MS (Method 1): Rₜ=0.61 min; MS (ESIpos): m/z=707 [M+H—HCl]⁺.

### Example 3

### N-alpha-{[trans-4-(Aminomethyl)cyclohexyl]carbonyl}-4-(6-{[2-(diethylamino)ethyl]carbamoyl}-2-methylpyridin-3-yl)-N-[4-(2H-tetrazol-5-yl)phenyl]-L-phenylalaninamide hydrochloride

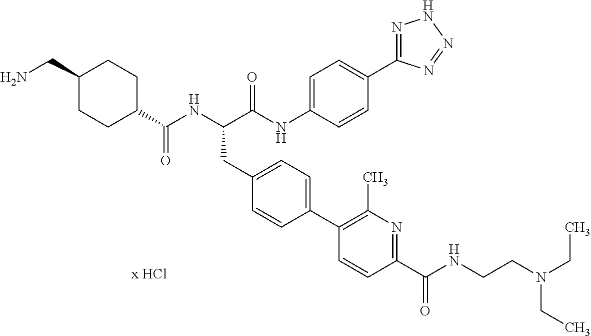

To a solution of 85 mg (0.095 mmol) of N-alpha-[(trans-4-{[(tert-butoxycarbonyl)amino]-methyl}cyclohexyl)carbonyl]-4-(6-{[2-(diethylamino)ethyl]carbamoyl}-2-methylpyridin-3-yl)-N-[4-(2H-tetrazol-5-yl)phenyl]-L-phenylalaninamide trifluoroacetate in 3 ml of dioxane was added 0.36 ml (1.43 mmol) of 4M hydrogen chloride in dioxane. The mixture was stirred at RT for 72 h. The precipitated product was filtered off with suction, washed with acetonitrile and dried under high vacuum. 58 mg (76% of theory) of the title compound were obtained.

LC-MS (Method 1): Rₜ=0.60 min; MS (ESIpos): m/z=681.4 [M+H—HCl]⁺.

¹H NMR (400 MHz, DMSO-d₆): δ=ppm 0.80-1.00 (m, 2H), 1.24 (t, 7H), 1.49 (br. s., 1H), 1.59 (d, 1H), 1.71-1.84 (m, 3H), 2.11-2.22 (m, 1H), 2.46 (s, 3H), 2.63 (t, 2H), 2.90-3.04 (m, 1H), 3.12-3.28 (m, 7H), 3.70 (q, 2H), 4.76 (m, 2H), 7.36 (d, 2H), 7.45 (d, 2H), 7.76 (d, 1H), 7.84 (d, 2H), 7.93 (m, 4H), 8.04 (d, 2H), 8.34 (d, 1H), 9.06 (t, 1H), 10.14 (br. s, 1H), 10.62 (br. s., 1H).

### Example 4

### N-alpha-{[trans-4-(Aminomethyl)cyclohexyl]carbonyl}-4-(2-methyl-6-{[(3S)-2-oxopiperidin-3-yl]carbamoyl}pyridin-3-yl)-N-[4-(2H-tetrazol-5-yl)phenyl]-L-phenylalaninamide hydrochloride

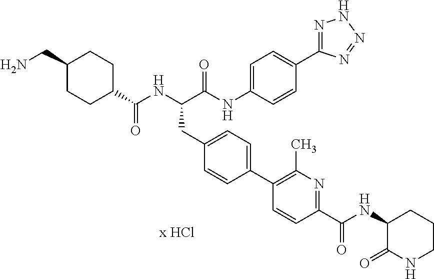

To a solution of 52.4 mg (0.06 mmol) of N-alpha-[(trans-4-{[(tert-butoxycarbonyl)amino]-methyl}cyclohexyl)carbonyl]-4-(2-methyl-6-{[(3S)-2-oxopiperidin-3-yl]carbamoyl}pyridin-3-yl)-N-[4-(2H-tetrazol-5-yl)phenyl]-L-phenylalaninamide trifluoroacetate in 2 ml of dioxane was added 0.22 ml (0.88 mmol) of 4M hydrogen chloride in dioxane. The mixture was stirred at RT for 72 h. The precipitated product was filtered off with suction, washed with dioxane and dried under high vacuum. 32 mg (71% of theory) of the title compound were obtained.

LC-MS (Method 1): Rₜ=0.68 min; MS (ESIpos): m/z=679 [M+H—HCl]⁺.

¹H NMR (400 MHz, DMSO-d₆): δ=ppm 0.84-1.00 (m, 2H), 1.09-1.34 (m, 2H), 1.41-1.52 (m, 1H), 1.53-1.62 (m, 1H), 1.67-1.87 (m, 6H), 2.10-2.24 (m, 2H), 2.46 (s, 3H), 2.63 (t, 2H), 2.89-3.03 (m, 1H), 3.13-3.25 (m, 3H), 4.30-4.39 (m, 1H), 4.77 (m, 1H), 7.36 (d, 2H), 7.44 (d, 2H), 7.73 (br. s., 1H), 7.76 (d, 1H), 7.84 (m, 4H), 7.93 (d, 1H), 8.03 (d, 2H), 8.30 (d, 1H), 8.83 (d, 1H), 10.58 (br. s., 1H).

### Example 5

### N-alpha-{[trans-4-(Aminomethyl)cyclohexyl]carbonyl}-4-[4-methyl-6-(piperidin-4-ylcarbamoyl)pyridin-3-yl]-N-[4-(1H-tetrazol-5-yl)phenyl]-L-phenylalaninamide hydrochloride

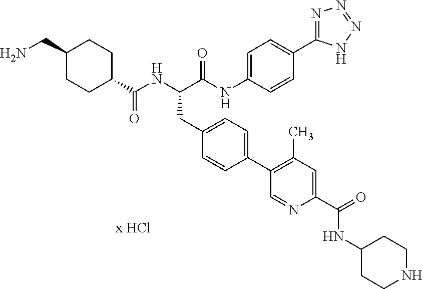 [SMILES: Cc1cc(C(=O)NC2CCNCC2)ncc1-c1ccc(C[C@H](NC(=O)[C@H]2CC[C@H](CN)CC2)C(=O)Nc2ccc(-c3nnn[nH]3)cc2)cc1]

To a solution of 32.6 mg (37.7 μmol) of tert-butyl 4-{[(5-{4-[(2S)-2-{[(trans-4-{[(tert-butoxycarbonyl)amino]methyl}cyclohexyl)carbonyl]amino}-3-oxo-3-{[4-(1H-tetrazol-5-yl)-phenyl]amino}propyl]phenyl}-4-methylpyridin-2-yl)carbonyl]amino}piperidine-1-carboxylate formate in 2 ml of dichloromethane were added 94 μl (0.38 mmol) of 4M hydrogen chloride in dioxane. The mixture was stirred at 35° C. for 5 h. The reaction mixture was admixed with acetonitrile. The precipitated product was filtered off with suction, washed with acetonitrile and dried under high vacuum. 21 mg (72% of theory) of the title compound were obtained.

¹H NMR (300 MHz, DMSO-d₆): δ=ppm 0.91 (m, 2H), 1.08-1.34 (m, 2H), 1.39-1.62 (m, 2H), 1.65-2.01 (m, 8H), 2.08-2.21 (m, 1H), 2.30 (s, 3H), 2.60 (m, 2H), 2.90-3.08 (m, 3H), 3.13-3.21 (m, 1H), 3.29 (m, 2H), 4.09 (m, 1H), 4.76 (m, 1H), 7.34 (d, 2H), 7.46 (d, 2H), 7.84 (d, 2H), 7.93 (br. s., 2H), 7.97 (s, 1H), 8.03 (d, 2H), 8.31 (d, 1H), 8.37 (s, 1H), 8.65-8.77 (m, 1H), 8.81 (d, 1H), 8.93-9.07 (m, 1H), 10.64 (s, 1H)

LC-MS (Method 4) Rₜ=0.61 min; MS (ESIpos): m/z=665.4 [M+H—HCl]⁺.

### Example 6

### N-alpha-{[trans-4-(Aminomethyl)cyclohexyl]carbonyl}-4-{6-[(3,5-dimethylpiperazin-1-yl)carbonyl]-4-methylpyridin-3-yl}-N-[4-(1H-tetrazol-5-yl)phenyl]-L-phenylalaninamide hydrochloride

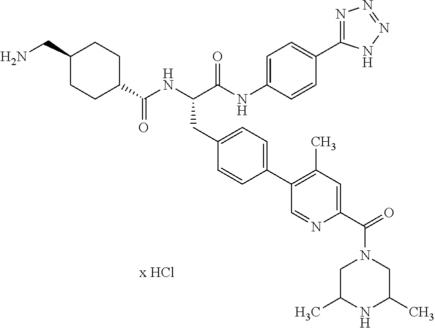

To a solution of 26.6 mg (34.15 μmol) of N-alpha-[(trans-4-{[(tert-butoxycarbonyl)amino]-methyl}cyclohexyl)carbonyl]-4-{6-[(3,5-dimethylpiperazin-1-yl)carbonyl]-4-methylpyridin-3-yl}-N-[4-(1H-tetrazol-5-yl)phenyl]-L-phenylalaninamide in 2 ml of dichloromethane were added 85 μl (0.34 mmol) of 4M hydrogen chloride in dioxane. The mixture was stirred at RT for 24 h. The reaction mixture was admixed with acetonitrile. The precipitated product was filtered off with suction, washed with acetonitrile and dried under high vacuum. 9 mg (32% of theory) of the title compound were obtained.

¹H NMR (300 MHz, DMSO-d₆): δ=ppm 0.81-1.01 (m, 2H), 1.16 (d, 3H), 1.21-1.29 (m, 2H), 1.32 (d, 3H), 1.40-1.51 (m, 1H), 1.53-1.63 (m, 1H), 1.65-1.82 (m, 3H), 2.08-2.20 (m, 1H), 2.27 (s, 3H), 2.58-2.67 (m, 2H), 2.80-2.90 (m, 2H), 3.15 (dd, 1H), 3.28-3.40 (m, 1H), 4.05-4.16 (m, 1H), 4.56-4.67 (m, 1H), 4.70-4.80 (m, 1H), 7.35 (d, 2H), 7.44 (d, 2H), 7.61 (s, 1H), 7.82 (m, 5H), 7.99 (d, 2H), 8.30 (d, 1H), 8.34 (s, 1H), 9.03-9.21 (m, 1H), 9.45-9.58 (m, 1H), 10.53 (s, 1H).

LC-MS (Method 4) Rₜ=0.58 min; MS (ESIpos): m/z=679 [M+H—HCl]⁺.

### Example 7

### N-alpha-{[trans-4-(Aminomethyl)cyclohexyl]carbonyl}-4-(6-{[4-(diethylamino)cyclohexyl]-carbamoyl}-4-methylpyridin-3-yl)-N-[4-(1H-tetrazol-5-yl)phenyl]-L-phenylalaninamide hydrochloride

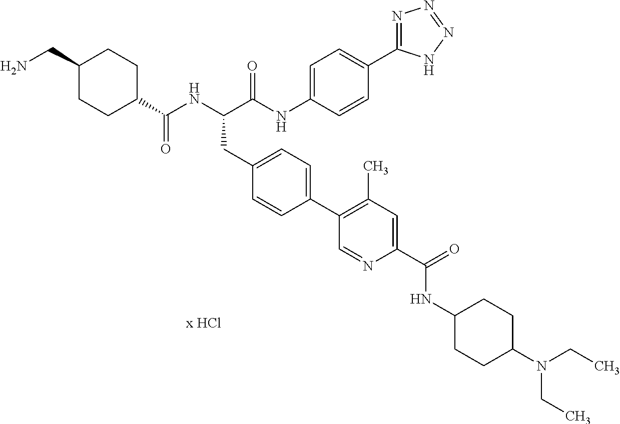

To a solution of 26.0 mg (31.1 μmol) of N-alpha-[(trans-4-{[(tert-butoxycarbonyl)-amino]methyl}cyclohexyl)carbonyl]-4-(6-{[4-(diethylamino)cyclohexyl]carbamoyl}-4-methylpyridin-3-yl)-N-[4-(1H-tetrazol-5-yl)phenyl]-L-phenylalaninamide in 2 ml of dichloromethane were added 78 μl (0.38 mmol) of 4M hydrogen chloride in dioxane. The mixture was stirred at RT for 24 h. The reaction mixture was admixed with acetonitrile. The precipitated product was filtered off with suction, washed with acetonitrile and dried under high vacuum. 17 mg (61% of theory, 93% purity) of the title compound were obtained.

¹H NMR (300 MHz, DMSO-d₆): δ=ppm 0.81-0.96 (m, 2H), 1.09-1.18 (m, 1H), 1.21-1.30 (m, 12H), 1.43-1.63 (m, 6H), 1.74 (m, 6H), 1.86-2.01 (m, 5H), 2.06-2.21 (m, 3H), 2.30 (s, 3H), 2.58-2.66 (m, 2H), 2.93-3.34 (m, 13H), 3.80-3.88 (m, 1H), 4.70-4.80 (m, 1H), 7.30-7.38 (m, 3H), 7.44 (d, 2H), 7.81-7.92 (m, 5H), 7.96 (s, 1H), 8.02 (d, 2H), 8.27-8.40 (m, 2H), 8.58 (d, 1H), 9.87-9.97 (m, 1H), 10.58-10.65 (m, 1H)

LC-MS (Method 4) Rₜ=0.67 min; MS (ESIpos): m/z=735 [M+H—HCl]⁺.

### Example 8

### N-alpha-{[trans-4-(Aminomethyl)cyclohexyl]carbonyl}-4-{6-[(3-fluoropiperidin-4-yl)carbamoyl]-4-methylpyridin-3-yl}-N-[4-(1H-tetrazol-5-yl)phenyl]-L-phenylalaninamide hydrochloride

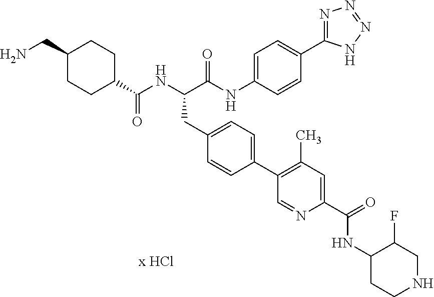

To a solution of 13.2 mg (14.9 μmol) of tert-butyl 4-{[(5-{4-[(2S)-2-{[(trans-4-{[(tert-butoxy-carbonyl)amino]methyl}cyclohexyl)carbonyl]amino}-3-oxo-3-{[4-(1H-tetrazol-5-yl)phenyl]-amino}propyl]phenyl}-4-methylpyridin-2-yl)carbonyl]amino}-3-fluoropiperidine-1-carboxylate in 1 ml of dichloromethane were added 37 μl (0.15 mmol) of 4M hydrogen chloride in dioxane. The mixture was stirred at 35° C. for 5 h. The reaction mixture was admixed with acetonitrile. The precipitated product was filtered off with suction, washed with acetonitrile and dried under high vacuum. 8 mg (71% of theory) of the title compound were obtained.

¹H NMR (300 MHz, DMSO-d₆): δ=ppm 0.82-1.02 (m, 2H), 1.10-1.36 (m, 2H), 1.40-1.52 (m, 1H), 1.53-1.63 (m, 1H), 1.68-2.01 (m, 7H), 2.08-2.21 (m, 1H), 2.30 (d, 3H), 2.56-2.68 (m, 2H), 2.96-3.04 (m, 2H), 3.16 (dd, 1H), 3.25-3.34 (m, 2H), 4.71-4.82 (m, 1H), 7.29-7.38 (m, 2H), 7.42-7.49 (m, 2H), 7.83 (m, 5H), 7.94-8.06 (m, 3H), 8.29 (d, 1H), 8.39 (d, 1H), 8.61-8.67 (m, 1H), 8.79-8.84 (m, 1H), 10.56 (s, 1H)

LC-MS (Method 4) Rₜ=0.62 min; MS (ESIpos): m/z=683 [M+H—HCl]⁺.

### Example 9

### N-alpha-{[trans-4-(Aminomethyl)cyclohexyl]carbonyl}-4-(6-{[3-(diethylamino)propyl]-carbamoyl}-4-methylpyridin-3-yl)-N-[4-(1H-tetrazol-5-yl)phenyl]-L-phenylalaninamide hydrochloride

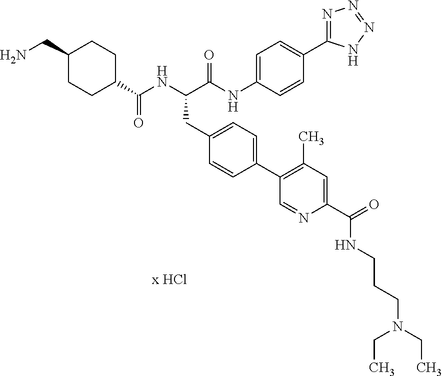

To a solution of 19 mg (23.9 μmol) of N-alpha-[(trans-4-{[(tert-butoxycarbonyl)amino]-methyl}cyclohexyl)carbonyl]-4-(6-{[3-(diethylamino)propyl]carbamoyl}-4-methylpyridin-3-yl)-N-[4-(1H-tetrazol-5-yl)phenyl]-L-phenylalaninamide in 1.5 ml of dichloromethane were added 60 μl (0.24 mmol) of 4M hydrogen chloride in dioxane. The mixture was stirred at RT for 24 h. The reaction mixture was admixed with acetonitrile. The precipitated product was filtered off with suction, washed with acetonitrile and dried under high vacuum. 11 mg (59% of theory) of the title compound were obtained.

¹H NMR (400 MHz, DMSO-d₆): δ=ppm 0.83-0.98 (m, 2H), 1.17 (m, 6H), 1.23-1.31 (m, 1H), 1.42-1.52 (m, 1H), 1.53-1.61 (m, 1H), 1.68-1.83 (m, 3H), 1.88-1.98 (m, 2H), 2.10-2.20 (m, 1H), 2.30 (s, 3H), 2.57-2.64 (m, 2H), 2.97 (dd, 1H), 3.07 (td, 7H), 3.16 (dd, 2H), 3.34-3.99 (m, 4H), 3.39 (d, 2H), 4.72-4.79 (m, 1H), 7.35 (d, 2H), 7.45 (d, 2H), 7.84 (d, 2H), 7.90 (br. s., 3H), 7.97 (s, 1H), 8.03 (d, 2H), 8.31 (d, 1H), 8.37 (s, 1H), 8.96-9.04 (m, 1H), 10.06-10.17 (m, 1H), 10.61 (s, 1H)

LC-MS (Method 4) Rₜ=0.64 min; MS (ESIpos): m/z=695.4 [M+H—HCl]⁺.

### Example 10

### N-alpha-{[trans-4-(Aminomethyl)cyclohexyl]carbonyl}-4-{6-[(1R,5S)-8-azabicyclo[3.2.1]oct-3-ylcarbamoyl]-4-methylpyridin-3-yl}-N-[4-(1H-tetrazol-5-yl)phenyl]-L-phenylalaninamide hydrochloride

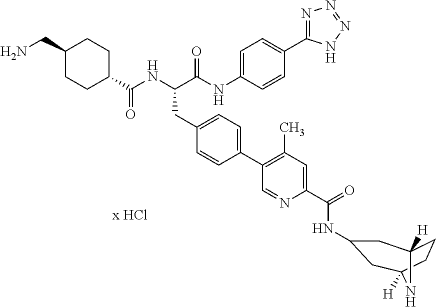 [SMILES: Cc1cc(C(=O)NC2C[C@@H]3CC[C@H](C2)NC3)ncc1-c1ccc(C[C@H](NC(=O)[C@H]2CC[C@H](CN)CC2)C(=O)Nc2ccc(-c3nnn[nH]3)cc2)cc1]

To a solution of 33.4 mg (37.5 μmol) of tert-butyl (1R,5S)-3-{[(5-{4-[(2S)-2-{[(trans-4-{[(tert-butoxycarbonyl)amino]methyl}cyclohexyl)carbonyl]amino}-3-oxo-3-{[4-(1H-tetrazol-5-yl)-phenyl]amino}propyl]phenyl}-4-methylpyridin-2-yl)carbonyl]amino}-8-azabicyclo[3.2.1]octane-1-carboxylate formate in 2.3 ml of dichloromethane were added 94 μl (0.37 mmol) of 4M hydrogen chloride in dioxane. The mixture was stirred at RT for 24 h. The reaction mixture was admixed with acetonitrile. The precipitated product was filtered off with suction, washed with acetonitrile and dried under high vacuum. 23 mg (77% of theory) of the title compound were obtained.

¹H NMR (400 MHz, DMSO-d₆): δ=ppm 0.82-1.01 (m, 2H), 1.12-1.33 (m, 2H), 1.41-1.52 (m, 1H), 1.54-1.62 (m, 1H), 1.68-1.82 (m, 3H), 2.14 (m, 9H), 2.25-2.35 (m, 5H), 2.58-2.64 (m, 2H), 2.98 (dd, 1H), 3.16 (dd, 1H), 3.93-4.10 (m, 8H), 4.72-4.80 (m, 1H), 7.35 (m, 2H), 7.45 (d, 2H), 7.83 (d, 2H), 7.87 (br. s., 3H), 7.96 (s, 1H), 8.02 (d, 2H), 8.27-8.34 (m, 1H), 8.40 (s, 1H), 8.60-8.64 (m, 1H), 8.85-8.96 (m, 1H), 9.01-9.18 (m, 1H), 10.59 (s, 1H)

LC-MS (Method 4) Rₜ=0.63 min; MS (ESIpos): m/z=691 [M+H—HCl]⁺.

### Example 11

### N-alpha-{[trans-4-(Aminomethyl)cyclohexyl]carbonyl}-4-[4-methyl-6-(3-oxa-9-azabicyclo[3.3.1]non-7-ylcarbamoyl)pyridin-3-yl]-N-[4-(1H-tetrazol-5-yl)phenyl]-L-phenylalaninamide hydrochloride

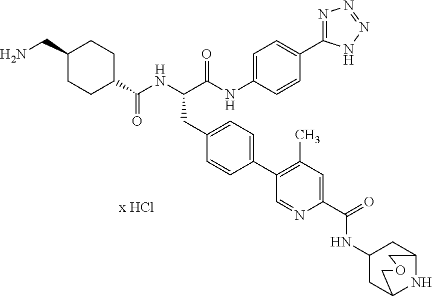

To a solution of 32.7 mg (36.0 μmol) of tert-butyl 7-{[(5-{4-[(2S)-2-{[(trans-4-{[(tert-butoxy-carbonyl)amino]methyl}cyclohexyl)carbonyl]amino}-3-oxo-3-{[4-(1H-tetrazol-5-yl)phenyl]-amino}propyl]phenyl}-4-methylpyridin-2-yl)carbonyl]amino}-3-oxa-9-azabicyclo[3.3.1]nonane-1-carboxylate formate in 2.2 ml of dichloromethane were added 90 μl (0.36 mmol) of 4M hydrogen chloride in dioxane. The mixture was stirred at 35° C. for 5 h. The reaction mixture was admixed with acetonitrile. The precipitated product was filtered off with suction, washed with acetonitrile and dried under high vacuum. 23 mg (79% of theory) of the title compound were obtained.

¹H NMR (300 MHz, DMSO-d₆): δ=ppm 0.82-1.01 (m, 2H), 1.10-1.33 (m, 2H), 1.40-1.52 (m, 1H), 1.53-1.63 (m, 1H), 1.67-1.91 (m, 5H), 2.09-2.21 (m, 1H), 2.29 (s, 3H), 2.61 (m, 2H), 2.97 (dd, 1H), 3.16 (dd, 1H), 3.92-4.09 (m, 5H), 4.51-4.61 (m, 1H), 4.70-4.80 (m, 1H), 7.34 (d, 2H), 7.44 (d, 2H), 7.79-7.91 (m, 5H), 7.99 (d, 2H), 8.03 (s, 1H), 8.29 (d, 1H), 8.36 (s, 1H), 9.30-9.42 (m, 1H), 9.62 (d, 1H), 9.73-9.83 (m, 1H), 10.58 (s, 1H)

LC-MS (Method 4) Rₜ=0.65 min; MS (ESIpos): m/z=707.4 [M+H—HCl]⁺.

### Example 12

### N-alpha-{[trans-4-(Aminomethyl)cyclohexyl]carbonyl}-4-(6-{[3-(dimethylamino)propyl]-carbamoyl}-4-methylpyridin-3-yl)-N-[4-(1H-tetrazol-5-yl)phenyl]-L-phenylalaninamide hydrochloride

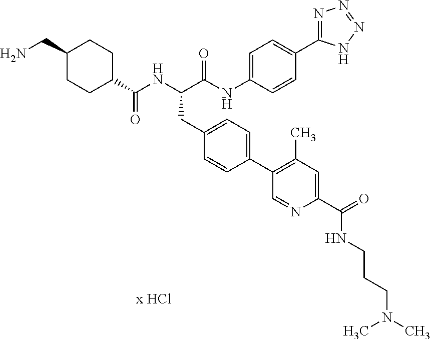

To a solution of 14.0 mg (18.3 μmol) of N-alpha-[(trans-4-{[(tert-butoxycarbonyl)-amino]methyl}cyclohexyl)carbonyl]-4-(6-{[3-(dimethylamino)propyl]carbamoyl}-4-methylpyridin-3-yl)-N-[4-(1H-tetrazol-5-yl)phenyl]-L-phenylalaninamide in 1.1 ml of dichloromethane were added 46 μl (0.18 mmol) of 4M hydrogen chloride in dioxane. The mixture was stirred at RT overnight. The reaction mixture was admixed with acetonitrile. The precipitated product was filtered off with suction, washed with acetonitrile and dried under high vacuum. 11 mg (78% of theory) of the title compound were obtained.

¹H NMR (400 MHz, DMSO-d₆): δ=ppm 0.81-0.99 (m, 2H), 1.12-1.32 (m, 2H), 1.42-1.53 (m, 1H), 1.54-1.62 (m, 1H), 1.69-1.81 (m, 4H), 1.87-1.96 (m, 2H), 2.10-2.19 (m, 1H), 2.30 (s, 3H), 2.31-2.33 (m, 1H), 2.58-2.64 (m, 2H), 2.73 (s, 3H), 2.74 (s, 3H), 2.88-2.92 (m, 1H), 2.97 (dd, 1H), 3.02-3.10 (m, 2H), 3.15 (dd, 1H), 3.35-3.40 (m, 4H), 4.70-4.80 (m, 1H), 7.35 (d, 2H), 7.45 (d, 2H), 7.82 (m, 5H), 7.95-8.03 (m, 4H), 8.29 (d, 1H), 8.37 (s, 1H), 8.95-9.02 (m, 1H), 9.82-9.93 (m, 1H), 10.53 (s, 1H)

LC-MS (Method 4) Rₜ=0.61 min; MS (ESIpos): m/z=667 [M+H—HCl]⁺.

### Example 13

### N-alpha-{[trans-4-(Aminomethyl)cyclohexyl]carbonyl}-4-(4-methyl-6-{[trans-4-(morpholin-4-yl)cyclohexyl]carbamoyl}pyridin-3-yl)-N-[4-(1H-tetrazol-5-yl)phenyl]-L-phenylalaninamide hydrochloride

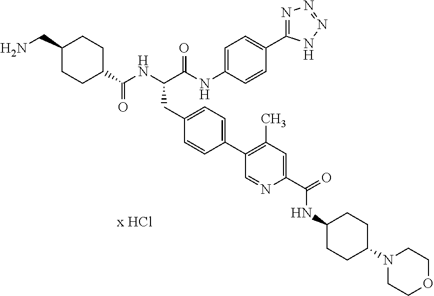

To a solution of 20.0 mg (23.6 μmol) of N-alpha-[(trans-4-{[(tert-butoxycarbonyl)-amino]methyl}cyclohexyl)carbonyl]-4-(4-methyl-6-{[trans-4-(morpholin-4-yl)cyclohexyl]-carbamoyl}pyridin-3-yl)-N-[4-(1H-tetrazol-5-yl)phenyl]-L-phenylalaninamide in 1.4 ml of dichloromethane were added 29 μl (0.12 mmol) of 4M hydrogen chloride in dioxane. The mixture was then stirred at 35° C. for 5 h. The reaction mixture was admixed with acetonitrile. The precipitated product was filtered off with suction, washed with acetonitrile and dried under high vacuum. 12 mg (60% of theory) of the title compound were obtained.

¹H NMR (300 MHz, DMSO-d₆): δ=ppm 0.83-1.01 (m, 2H), 1.08-1.33 (m, 3H), 1.40-1.82 (m, 11H), 1.93 (m, 2H), 2.13-2.24 (m, 3H), 2.30 (s, 3H), 2.57-2.67 (m, 2H), 2.98 (dd, 1H), 3.04-3.21 (m, 4H), 3.38 (d, 2H), 3.80-4.00 (m, 4H), 4.70-4.81 (m, 1H), 7.34 (d, 2H), 7.45 (d, 2H), 7.84 (m, 5H), 7.94-8.06 (m, 3H), 8.30 (d, 1H), 8.36 (s, 1H), 8.62 (d, 1H), 10.59 (s, 1H), 10.98-11.14 (m, 1H)

LC-MS (Method 4) Rₜ=0.64 min; MS (ESIpos): m/z=749 [M+H—HCl]⁺.

### Example 14

### N-alpha-{[trans-4-(Aminomethyl)cyclohexyl]carbonyl}-4-{2-methyl-6-[(3S)-pyrrolidin-3-ylcarbamoyl]pyridin-3-yl}-N-(2-oxo-2,3-dihydro-1H-benzimidazol-5-yl)-L-phenylalaninamide hydrochloride

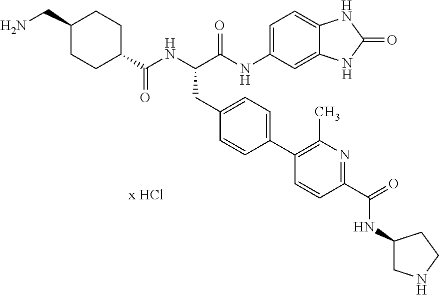 [SMILES: Cc1nc(C(=O)N[C@H]2CCNC2)ccc1-c1ccc(C[C@H](NC(=O)[C@H]2CC[C@H](CN)CC2)C(=O)Nc2ccc3[nH]c(=O)[nH]c3c2)cc1]

To a solution of 32.0 mg (38 μmol) of tert-butyl (3S)-3-({[5-(4-{(2S)-2-{[(trans-4-{[(tert-butoxycarbonyl)amino]methyl}cyclohexyl)carbonyl]amino}-3-oxo-3-[(2-oxo-2,3-dihydro-1H-benzimidazol-5-yl)amino]propyl}phenyl)-6-methylpyridin-2-yl]carbonyl}amino)pyrrolidine-1-carboxylate trifluoroacetate in 1.5 ml of dioxane were added 143 μl (0.57 mmol) of 4M hydrogen chloride in dioxane. The mixture was stirred at RT overnight. The reaction mixture was concentrated, taken up in 0.5 ml of dimethyl sulphoxide and a little acetonitrile and separated by means of preparative HPLC (acetonitrile/water gradient+0.1% TFA). The product-containing fractions were concentrated and dried under high vacuum. Subsequently, they were dissolved in methanol, admixed with 0.1 ml of 4N hydrogen chloride in dioxane and concentrated. The solid was dried under high vacuum. 19 mg (69% of theory) of the title compound were obtained.

¹H NMR (400 MHz, DMSO-d₆): δ=ppm 0.79-0.99 (m, 2H), 1.11-1.34 (m, 2H), 1.41-1.52 (m, 1H), 1.53-1.62 (m, 1H), 1.66-1.81 (m, 3H), 1.99-2.09 (m, 1H), 2.11-2.19 (m, 1H), 2.21-2.30 (m, 1H), 2.47 (s, 3H), 2.60-2.65 (m, 2H), 2.95 (dd, 1H), 3.10 (dd, 1H), 3.19-3.30 (m, 2H), 3.33-3.47 (m, 2H), 4.62-4.74 (m, 1H), 6.84 (d, 1H), 7.04 (d, 1H), 7.34 (d, 2H), 7.41 (m, 3H), 7.75 (d, 1H), 7.84 (br. s, 3H), 7.91 (d, 1H), 8.22 (d, 1H), 8.97 (d, 1H), 9.08-9.27 (m, 2H), 10.00-10.05 (m, 1H), 10.52 (s, 1H), 10.57 (s, 1H)

LC-MS (Method 1): Rₜ=0.49 min; MS (ESIpos): m/z=639 [M+H—HCl]⁺.

### Example 15

### N-alpha-{[trans-4-(Aminomethyl)cyclohexyl]carbonyl}-4-{2-methyl-6-[(3S)-pyrrolidin-3-ylcarbamoyl]pyridin-3-yl}-N-[4-(2H-tetrazol-5-yl)phenyl]-L-phenylalaninamide hydrochloride

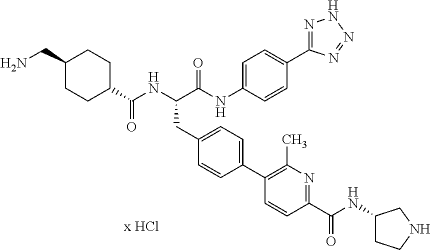

To a solution of 72.4 mg (75 μmol) of tert-butyl (3S)-3-{[(5-{4-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]phenyl}-6-methylpyridin-2-yl)carbonyl]amino}pyrrolidine-1-carboxylate trifluoroacetate in 3.5 ml of dioxane were added 281 μl (1.13 mmol) of 4M hydrogen chloride in dioxane. The mixture was stirred at RT for 6 days. The reaction mixture was concentrated, taken up in 1 ml of dimethylformamide and 3 ml of acetonitrile and separated by means of preparative HPLC (acetonitrile/water gradient+0.1% TFA). The product-containing fractions were concentrated and dried under high vacuum. Subsequently, they were dissolved in methanol, admixed with 0.1 ml of 4N hydrogen chloride in dioxane and concentrated. The solid was dried under high vacuum. 19 mg (35% of theory) of the title compound were obtained.

¹H NMR (400 MHz, DMSO-d₆): δ=ppm 0.85-1.01 (m, 2H), 1.13-1.34 (m, 2H), 1.43-1.54 (m, 1H), 1.56-1.63 (m, 1H), 1.75 (m, 3H), 1.99-2.09 (m, 1H), 2.12-2.20 (m, 1H), 2.22-2.29 (m, 1H), 2.47 (s, 3H), 2.63 (m, 2H), 2.99 (dd, 1H), 3.17 (dd, 1H), 3.20-3.30 (m, 2H), 3.40 (m, 2H), 4.63-4.69 (m, 1H), 4.72-4.80 (m, 1H), 7.35 (d, 2H), 7.44 (d, 2H), 7.76 (d, 1H), 7.84 (d, 2H), 7.89 (br. s., 2H), 7.91-7.94 (m, 1H), 8.03 (d, 2H), 8.33 (d, 1H), 8.98 (d, 1H), 9.16-9.34 (m, 2H), 10.59 (s, 1H)

LC-MS (Method 1): Rₜ=0.56 min; MS (ESIpos): m/z=651 [M+H—HCl]⁺.

### Example 16

### N-alpha-{[trans-4-(Aminomethyl)cyclohexyl]carbonyl}-4-[6-(3-azabicyclo[3.1.0]hex-6-ylcarbamoyl)-2-methylpyridin-3-yl]-N-[4-(2H-tetrazol-5-yl)phenyl]-L-phenylalaninamide hydrochloride

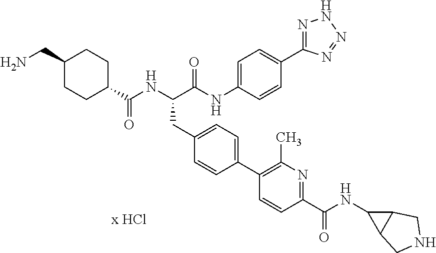 [SMILES: Cc1nc(C(=O)NC2C3CNCC32)ccc1-c1ccc(C[C@H](NC(=O)[C@H]2CC[C@H](CN)CC2)C(=O)Nc2ccc(-c3nn[nH]n3)cc2)cc1]

To a solution of 85.5 mg (88 μmol) of tert-butyl 6-{[(5-{4-[(2S)-2-{[(trans-4-{[(tert-butoxy-carbonyl)amino]methyl}cyclohexyl)carbonyl]amino}-3-oxo-3-{[4-(2H-tetrazol-5-yl)phenyl]-amino}propyl]phenyl}-6-methylpyridin-2-yl)carbonyl]amino}-3-azabicyclo[3.1.0]hexane-3-carboxylate trifluoroacetate in 3 ml of dioxane were added 328 μl (1.31 mmol) of 4M hydrogen chloride in dioxane. The mixture was stirred at RT overnight. The precipitated product was filtered off with suction, washed with acetonitrile and dried under high vacuum. 73 mg (99% of theory, 92% purity) of the title compound were obtained.

¹H NMR (400 MHz, DMSO-d₆): δ=ppm 0.85-0.99 (m, 2H), 1.09-1.33 (m, 2H), 1.43-1.54 (m, 1H), 1.55-1.63 (m, 1H), 1.74 (t, 3H), 2.10-2.20 (m, 3H), 2.44 (s, 3H), 2.63 (m, 2H), 2.98 (dd, 1H), 3.05 (m, 1H), 3.15 (dd, 1H), 3.31-3.44 (m, 5H), 4.69-4.79 (m, 2H), 7.34 (d, 2H), 7.44 (d, 2H), 7.74 (d, 1H), 7.84 (d, 2H), 7.89 (m, 4H), 8.03 (d, 2H), 8.32 (d, 1H), 8.78 (d, 1H), 9.00-9.12 (m, 1H), 9.53-9.67 (m, 1H), 10.60 (br. s., 1H)

LC-MS (Method 1): Rₜ=0.55 min; MS (ESIpos): m/z=663 [M+H—HCl]⁺.

### Example 17

### N-alpha-{[trans-4-(Aminomethyl)cyclohexyl]carbonyl}-4-{2-methyl-6-[(3R)-pyrrolidin-3-ylcarbamoyl]pyridin-3-yl}-N-[4-(2H-tetrazol-5-yl)phenyl]-L-phenylalaninamide hydrochloride

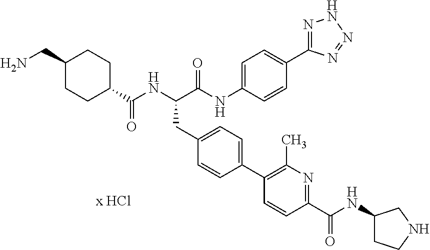 [SMILES: Cc1nc(C(=O)N[C@@H]2CCNC2)ccc1-c1ccc(C[C@H](NC(=O)[C@H]2CC[C@H](CN)CC2)C(=O)Nc2ccc(-c3nn[nH]n3)cc2)cc1]

To a solution of 70.2 mg (73 μmol) of tert-butyl (3R)-3-{[(5-{4-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]phenyl}-6-methylpyridin-2-yl)carbonyl]amino}pyrrolidine-1-carboxylate trifluoroacetate in 3.5 ml of dioxane were added 273 μl (1.09 mmol) of 4M hydrogen chloride in dioxane. The mixture was stirred at RT for 6 days. The reaction mixture was concentrated, taken up in 1 ml of dimethylformamide and 3 ml of acetonitrile and separated by means of preparative HPLC (acetonitrile/water gradient+0.1% TFA). The product-containing fractions were concentrated and dried under high vacuum. Subsequently, they were dissolved in methanol, admixed with 0.1 ml of 4N hydrogen chloride in dioxane and concentrated. The solid was dried under high vacuum. 32 mg (53% of theory) of the title compound were obtained.

¹H NMR (400 MHz, DMSO-d₆): δ=ppm 0.78-0.99 (m, 2H), 1.26 (br. s., 2H), 1.41-1.54 (m, 1H), 1.57 (br. s., 1H), 1.69-1.82 (m, 3H), 2.04 (d, 1H), 2.16 (br. s., 1H), 2.22-2.31 (m, 1H), 2.47 (s, 3H), 2.63 (m, 2H), 3.00 (dd, 1H), 4.62-4.69 (m, 1H), 4.73-4.80 (m, 1H), 7.35 (d, 2H), 7.44 (d, 2H), 7.76 (d, 1H), 7.84 (d, 2H), 7.91 (m, 3H), 8.03 (d, 2H), 8.33 (d, 1H), 8.98 (d, 1H), 9.16-9.37 (m, 2H), 10.59 (s, 1H)

LC-MS (Method 1): Rₜ=0.56 min; MS (ESIpos): m/z=651.4 [M+H—HCl]⁺.

### Example 18

### N-alpha-{[trans-4-(Aminomethyl)cyclohexyl]carbonyl}-4-(2-methyl-6-{[(2R,4R)-2-methylpiperidin-4-yl]carbamoyl}pyridin-3-yl)-N-[4-(2H-tetrazol-5-yl)phenyl]-L-phenylalaninamide hydrochloride

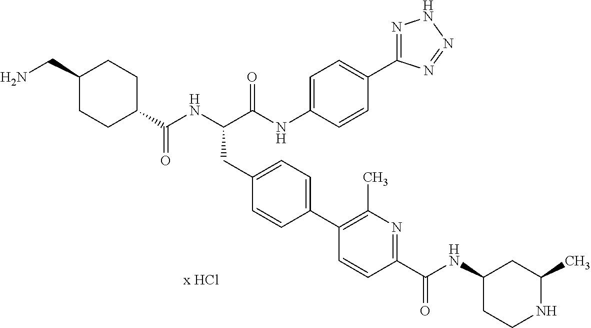 [SMILES: Cc1nc(C(=O)N[C@@H]2CCN[C@H](C)C2)ccc1-c1ccc(C[C@H](NC(=O)[C@H]2CC[C@H](CN)CC2)C(=O)Nc2ccc(-c3nn[nH]n3)cc2)cc1]

To a solution of 86.7 mg (87 μmol) of tert-butyl (2R,4R)-4-{[(5-{4-[(2S)-2-{[(trans-4-{[(tert-butoxycarbonyl)amino]methyl}cyclohexyl)carbonyl]amino}-3-oxo-3-{[4-(2H-tetrazol-5-yl)phenyl]amino}propyl]phenyl}-6-methylpyridin-2-yl)carbonyl]amino}-2-methylpiperidine-1-carboxylate trifluoroacetate in 3.0 ml of dioxane were added 327 μl (1.31 mmol) of 4M hydrogen chloride in dioxane. The mixture was stirred at RT for 72 h. The precipitated product was filtered off with suction, washed with a little dioxane and dried under high vacuum. The residue was recrystallized from methanol and acetonitrile, filtered with suction, washed with cold acetonitrile and dried again under high vacuum. 62 mg (95% of theory) of the title compound were obtained.

¹H NMR (400 MHz, DMSO-d₆): δ=ppm 0.84-1.00 (m, 2H), 1.10-1.23 (m, 1H), 1.28 (m, 4H), 1.44-1.52 (m, 1H), 1.58 (d, 1H), 1.63-1.90 (m, 5H), 1.93-2.07 (m, 2H), 2.16 (br. s., 1H), 2.46 (s, 3H), 2.63 (t, 2H), 2.94-3.06 (m, 2H), 3.15 (dd, 1H), 3.24-3.35 (m, 2H), 4.11 (d, 1H), 4.73-4.81 (m, 1H), 7.35 (d, 2H), 7.45 (d, 2H), 7.75 (d, 1H), 7.84 (d, 2H), 7.91 (m, 4H), 8.04 (d, 2H), 8.33 (d, 1H), 8.64 (d, 1H), 8.82-8.95 (m, 1H), 9.11-9.20 (m, 1H), 10.61 (br. s., 1H)

LC-MS (Method 1): Rₜ=0.59 min; MS (ESIpos): m/z=679 [M+H—HCl]⁺.

### Example 19

### N-alpha-{[trans-4-(Aminomethyl)cyclohexyl]carbonyl}-4-(2-methyl-6-{[(3R)-1-methylpyrrolidin-3-yl]carbamoyl}pyridin-3-yl)-N-[4-(2H-tetrazol-5-yl)phenyl]-L-phenylalaninamide hydrochloride

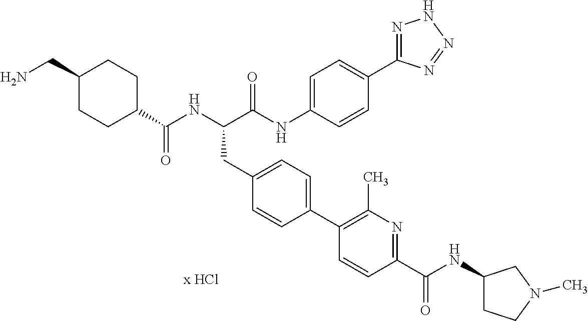

To a solution of 47.1 mg (54 μmol) of N-alpha-[(trans-4-{[(tert-butoxycarbonyl)amino]-methyl}cyclohexyl)carbonyl]-4-(2-methyl-6-{[(3R)-1-methylpyrrolidin-3-yl]carbamoyl}pyridin-3-yl)-N-[4-(2H-tetrazol-5-yl)phenyl]-L-phenylalaninamide trifluoroacetate in 2 ml of dioxane were added 201 μl (0.80 mmol) of 4M hydrogen chloride in dioxane. The mixture was stirred at RT for 2 days. The precipitated product was filtered off with suction, washed with a little dioxane and dried under high vacuum. 36 mg (91% of theory) of the title compound were obtained.

¹H NMR (400 MHz, DMSO-d₆): δ=ppm 0.76-1.02 (m, 2H), 1.11-1.33 (m, 2H), 1.43-1.52 (m, 1H), 1.55-1.62 (m, 1H), 1.74 (m, 3H), 1.98-2.08 (m, 1H), 2.11-2.22 (m, 2H), 2.26-2.34 (m, 1H), 2.46 (s, 3H), 2.59-2.67 (m, 2H), 2.82-2.89 (m, 3H), 2.94-3.09 (m, 2H), 3.10-3.18 (m, 2H), 3.24-3.39 (m, 2H), 4.71-4.85 (m, 2H), 7.35 (d, 2H), 7.44 (d, 2H), 7.75 (d, 1H), 7.83 (m, 5H), 7.91 (d, 1H), 8.02 (d, 2H), 8.28-8.35 (m, 1H), 10.55-10.60 (m, 1H)

LC-MS (Method 1): Rₜ=0.56 min; MS (ESIpos): m/z=665.4 [M+H—HCl]⁺.

### Example 20

### N-alpha-{[trans-4-(Aminomethyl)cyclohexyl]carbonyl}-4-{6-[(5,5-difluoropiperidin-3-yl)carbamoyl]-4-methylpyridin-3-yl}-N-[4-(1H-tetrazol-5-yl)phenyl]-L-phenylalaninamide hydrochloride

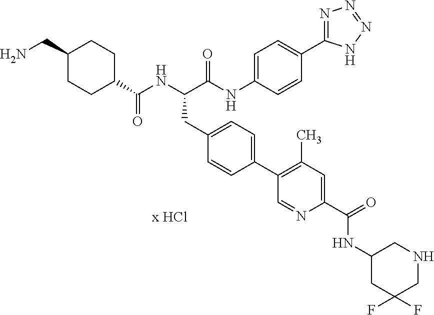

To a solution of 16.2 mg (18.0 μmol) of tert-butyl 5-{[(5-{4-[(2S)-2-{[(trans-4-{[(tert-butoxy-carbonyl)amino]methyl}cyclohexyl)carbonyl]amino}-3-oxo-3-{[4-(1H-tetrazol-5-yl)phenyl]-amino}propyl]phenyl}-4-methylpyridin-2-yl)carbonyl]amino}-3,3-difluoropiperidine-1-carboxylate in 1.0 ml of dichloromethane were added 45 μl (0.18 mmol) of 4M hydrogen chloride in dioxane. The mixture was then stirred at RT for 24 h. The reaction mixture was admixed with acetonitrile. The precipitated product was filtered off with suction, washed with acetonitrile and dried under high vacuum. 11 mg (77% of theory) of the title compound were obtained.

¹H NMR (300 MHz, DMSO-d₆): δ=ppm 0.82-1.00 (m, 2H), 1.11-1.33 (m, 3H), 1.40-1.52 (m, 1H), 1.53-1.62 (m, 1H), 1.68-1.81 (m, 3H), 2.10-2.21 (m, 1H), 2.31 (s, 3H), 2.59-2.65 (m, 2H), 2.93 (s, 2H), 2.96-3.02 (m, 1H), 3.08-3.14 (m, 2H), 3.27-3.35 (m, 1H), 3.44-3.52 (m, 1H), 4.43-4.54 (m, 1H), 4.69-4.80 (m, 1H), 7.35 (d, 2H), 7.45 (d, 2H), 7.76-7.86 (m, 5H), 7.98-8.04 (m, 3H), 8.32 (d, 1H), 8.40 (s, 1H), 9.16 (d, 1H), 10.56 (s, 1H)

LC-MS (Method 4) Rₜ=0.66 min; MS (ESIpos): m/z=701.4 [M+H—HCl]⁺.

### Example 21

### N-alpha-{[trans-4-(Aminomethyl)cyclohexyl]carbonyl}-4-(4-methyl-6-{[(3R)-2-oxopiperidin-3-yl]carbamoyl}pyridin-3-yl)-N-[4-(1H-tetrazol-5-yl)phenyl]-L-phenylalaninamide hydrochloride

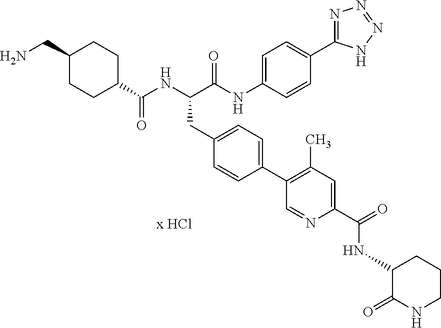

To a solution of 16.4 mg (21.1 μmol) of N-alpha-[(trans-4-{[(tert-butoxycarbonyl)amino]-methyl}cyclohexyl)carbonyl]-4-(4-methyl-6-{[(3R)-2-oxopiperidin-3-yl]carbamoyl}pyridin-3-yl)-N-[4-(1H-tetrazol-5-yl)phenyl]-L-phenylalaninamide in 1.3 ml of dichloromethane were added 26 μl (0.11 mmol) of 4M hydrogen chloride in dioxane. The mixture was then stirred at 35° C. for 5 h. The reaction mixture was admixed with acetonitrile. The precipitated product was filtered off with suction, washed with acetonitrile and dried under high vacuum. 10 mg (65% of theory) of the title compound were obtained.

¹H NMR (300 MHz, DMSO-d₆): δ=ppm 0.80-1.01 (m, 2H), 1.08-1.20 (m, 1H), 1.26 (t, 4H), 1.39-1.50 (m, 1H), 1.52-1.61 (m, 1H), 1.77 (d, 7H), 2.07-2.27 (m, 3H), 2.31 (s, 3H), 2.58-2.68 (m, 2H), 2.97 (dd, 1H), 3.17 (m, 4H), 4.28-4.38 (m, 1H), 4.71-4.82 (m, 1H), 7.36 (d, 2H), 7.43 (d, 2H), 7.68 (s, 1H), 7.77 (br. s., 3H), 7.83 (d, 2H), 7.95-8.05 (m, 3H), 8.25 (d, 1H), 8.39 (s, 1H), 8.81 (d, 1H), 10.53 (s, 1H)

LC-MS (Method 4) Rₜ=0.74 min; MS (ESIpos): m/z=679.4 [M+H—HCl]⁺.

### Example 22

### N-alpha-{[trans-4-(Aminomethyl)cyclohexyl]carbonyl}-4-{6-[(4-hydroxycyclohexyl)carbamoyl]-2-methylpyridin-3-yl}-N-[4-(2H-tetrazol-5-yl)phenyl]-L-phenylalaninamide hydrochloride

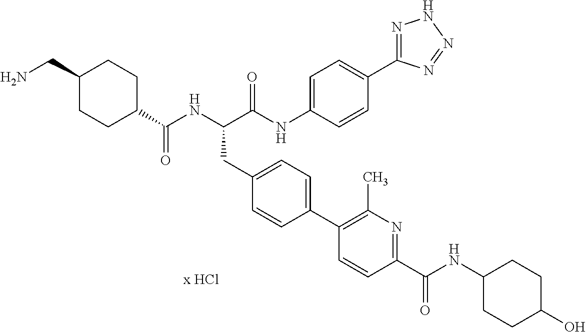

To a solution of 56.1 mg (63 μmol) of N-alpha-[(trans-4-{[(tert-butoxycarbonyl)amino]-methyl}cyclohexyl)carbonyl]-4-{6-[(4-hydroxycyclohexyl)carbamoyl]-2-methylpyridin-3-yl}-N-[4-(2H-tetrazol-5-yl)phenyl]-L-phenylalaninamide trifluoroacetate in 3.0 ml of dioxane were added 235 μl (0.94 mmol) of 4M hydrogen chloride in dioxane. The mixture was stirred at RT for 48 h. The precipitated product was filtered off with suction, washed with a little dioxane and dried under high vacuum. The residue was taken up in 1 ml of dimethylformamide and 3 ml of acetonitrile and separated by means of preparative HPLC (acetonitrile/water gradient+0.1% TFA). The product-containing fractions were concentrated and dried under high vacuum. Subsequently, they were dissolved in methanol, admixed with 0.1 ml of 4N hydrogen chloride in dioxane and concentrated. The solid was dried under high vacuum. 41 mg (83% of theory) of the title compound were obtained.

¹H NMR (400 MHz, DMSO-d₆): δ=ppm 0.82-0.99 (m, 2H), 1.11-1.21 (m, 1H), 1.27 (m, 3H), 1.47 (m, 4H), 1.69-1.90 (m, 7H), 2.11-2.19 (m, 1H), 2.45 (s, 3H), 2.63 (m, 2H), 2.97 (dd, 1H), 3.15 (dd, 1H), 3.38-3.47 (m, 1H), 3.69-3.81 (m, 1H), 4.75 (m, 1H), 7.34 (d, 2H), 7.44 (d, 2H), 7.74 (d, 1H), 7.84 (m, 5H), 7.91 (d, 1H), 8.03 (d, 2H), 8.30 (d, 2H), 10.57 (s, 1H)

LC-MS (Method 1): Rₜ=0.69 min; MS (ESIpos): m/z=680 [M+H—HCl]⁺.

### Example 23

### N-alpha-{[trans-4-(Aminomethyl)cyclohexyl]carbonyl}-4-{4-methyl-6-[(1-methyl-1H-pyrazol-3-yl)carbamoyl]pyridin-3-yl}-N-[4-(1H-tetrazol-5-yl)phenyl]-L-phenylalaninamide hydrochloride

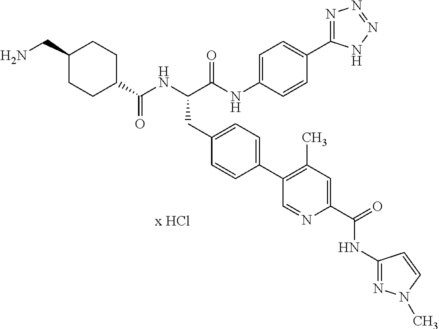

To a solution of 20.4 mg (26.8 μmol) of N-alpha-[(trans-4-{[(tert-butoxycarbonyl)-amino]methyl}cyclohexyl)carbonyl]-4-{4-methyl-6-[(1-methyl-1H-pyrazol-3-yl)carbamoyl]-pyridin-3-yl}-N-[4-(1H-tetrazol-5-yl)phenyl]-L-phenylalaninamide formate in 1.6 ml of dichloromethane were added 67 μl (0.27 mmol) of 4M hydrogen chloride in dioxane. The mixture was then stirred at RT for 24 h. The reaction mixture was admixed with acetonitrile. The precipitated product was filtered off with suction, washed with acetonitrile and dried under high vacuum. 17 mg (82% of theory) of the title compound were obtained.

¹H NMR (300 MHz, DMSO-d₆): δ=ppm 0.82-1.00 (m, 2H), 1.07-1.33 (m, 2H), 1.40-1.60 (m, 2H), 1.66-1.82 (m, 3H), 2.06-2.20 (m, 1H), 2.34 (s, 3H), 2.58-2.67 (m, 2H), 2.97 (dd, 1H), 3.16 (dd, 1H), 3.78 (s, 3H), 4.72-4.82 (m, 1H), 6.61 (d, 1H), 7.38 (d, 2H), 7.46 (d, 2H), 7.65 (d, 1H), 7.84 (m, 5H), 8.02 (d, 2H), 8.07 (s, 1H), 8.31 (d, 1H), 8.45 (s, 1H), 10.38 (s, 1H), 10.59 (s, 1H)

LC-MS (Method 4) Rₜ=0.82 min; MS (ESIpos): m/z=662 [M+H—HCl]⁺.

### Example 24

### N-alpha-{[trans-4-(Aminomethyl)cyclohexyl]carbonyl}-4-(6-{[3-(diethylamino)propyl]-carbamoyl}-2-methylpyridin-3-yl)-N-[4-(2H-tetrazol-5-yl)phenyl]-L-phenylalaninamide hydrochloride

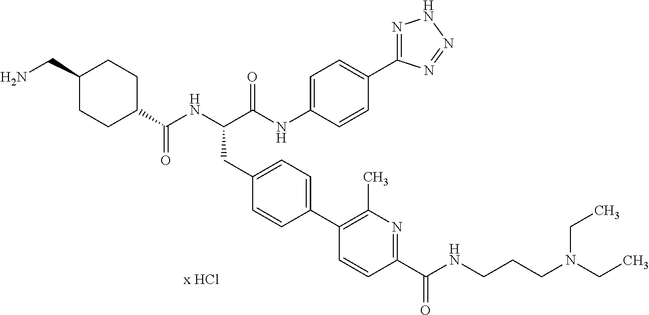

To a solution of 35.2 mg (39 μmol) of N-alpha-[(trans-4-{[(tert-butoxycarbonyl)amino]-methyl}cyclohexyl)carbonyl]-4-(6-{[3-(diethylamino)propyl]carbamoyl}-2-methylpyridin-3-yl)-N-[4-(2H-tetrazol-5-yl)phenyl]-L-phenylalaninamide trifluoroacetate in 1.5 ml of dioxane were added 145 μl (0.58 mmol) of 4M hydrogen chloride in dioxane. The mixture was stirred at RT for 6 days. The precipitated product was filtered off with suction, washed with a little dioxane and dried under high vacuum. The residue was concentrated, taken up in 1 ml of dimethylformamide and 3 ml of acetonitrile and separated by means of preparative HPLC (acetonitrile/water gradient+0.1% TFA). The product-containing fractions were concentrated and dried under high vacuum. Subsequently, they were dissolved in methanol, admixed with 0.1 ml of 4N hydrogen chloride in dioxane and concentrated. The solid was dried under high vacuum. 15 mg (50% of theory) of the title compound were obtained.

¹H NMR (400 MHz, DMSO-d₆): δ=ppm 0.87-1.00 (m, 2H), 1.20 (t, 6H), 1.23-1.33 (m, 1H), 1.43-1.54 (m, 1H), 1.56-1.63 (m, 1H), 1.70-1.82 (m, 3H), 1.89-2.00 (m, 2H), 2.12-2.21 (m, 1H), 2.45 (s, 3H), 2.60-2.68 (m, 2H), 2.98 (dd, 1H), 3.10 (m, 7H), 3.41 (m, 2H), 4.71-4.80 (m, 1H), 7.35 (d, 2H), 7.44 (d, 2H), 7.75 (d, 1H), 7.81-7.89 (m, 5H), 7.92 (d, 1H), 8.03 (d, 2H), 8.32 (d, 1H), 8.86-8.92 (m, 1H), 9.90-10.02 (m, 1H), 10.57 (s, 1H)

LC-MS (Method 1): Rₜ=0.60 min; MS (ESIpos): m/z=695.4 [M+H—HCl]⁺.

### Example 25

### N-alpha-{[trans-4-(Aminomethyl)cyclohexyl]carbonyl}-4-{4-methyl-6-[(3-methylpiperazin-1-yl)carbonyl]pyridin-3-yl}-N-[4-(1H-tetrazol-5-yl)phenyl]-L-phenylalaninamide hydrochloride

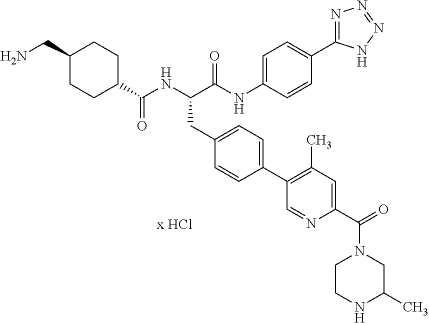

To a solution of 16.8 mg (22.0 μmol) of N-alpha-[(trans-4-{[(tert-butoxycarbonyl)-amino]methyl}cyclohexyl)carbonyl]-4-{4-methyl-6-[(3-methylpiperazin-1-yl)carbonyl]pyridin-3-yl}-N-[4-(1H-tetrazol-5-yl)phenyl]-L-phenylalaninamide formate in 1.3 ml of dichloromethane were added 55 μl (0.22 mmol) of 4M hydrogen chloride in dioxane. The mixture was then stirred at RT for 24 h. The reaction mixture was admixed with acetonitrile. The precipitated product was filtered off with suction, washed with acetonitrile and dried under high vacuum. 8 mg (47% of theory) of the title compound were obtained.

¹H NMR (300 MHz, DMSO-d₆): δ=ppm 0.79-0.98 (m, 4H), 1.01-1.05 (m, 1H), 1.11-1.30 (m, 2H), 1.37-1.47 (m, 1H), 1.51-1.60 (m, 1H), 1.65-1.81 (m, 3H), 2.08-2.19 (m, 1H), 2.26 (s, 3H), 2.61-2.66 (m, 2H), 2.69-2.73 (m, 2H), 2.91-3.03 (m, 2H), 3.13 (dd, 2H), 3.60-3.67 (m, 1H), 4.28-4.38 (m, 1H), 4.69-4.80 (m, 1H), 7.31-7.37 (m, 2H), 7.40-7.45 (m, 2H), 7.49 (s, 1H), 7.59 (d, 2H), 7.88 (d, 2H), 8.14-8.22 (m, 2H), 8.30-8.34 (m, 1H), 10.13 (s, 1H)

LC-MS (Method 4) Rₜ=0.65 min; MS (ESIpos): m/z=665.4 [M+H—HCl]⁺.

### Example 26

### N-alpha-{[trans-4-(Aminomethyl)cyclohexyl]carbonyl}-4-(2-methyl-6-{[(3R)-2-oxopyrrolidin-3-yl]carbamoyl}pyridin-3-yl)-N-[4-(2H-tetrazol-5-yl)phenyl]-L-phenylalaninamide hydrochloride

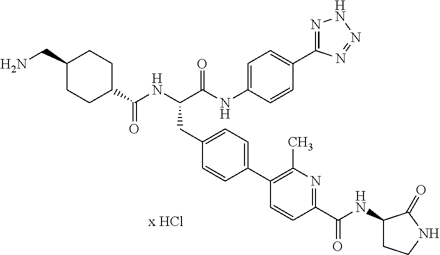

To a solution of 63.1 mg (72 μmol) of N-alpha-[(trans-4-{[(tert-butoxycarbonyl)amino]-methyl}cyclohexyl)carbonyl]-4-(2-methyl-6-{[(3R)-2-oxopyrrolidin-3-yl]carbamoyl}pyridin-3-yl)-N-[4-(2H-tetrazol-5-yl)phenyl]-L-phenylalaninamide trifluoroacetate in 3 ml of dioxane were added 269 μl (1.08 mmol) of 4M hydrogen chloride in dioxane. The mixture was stirred at RT for 48 h. The precipitated product was filtered off with suction, washed with a little dioxane and dried under high vacuum. The residue was concentrated, taken up in 1 ml of dimethylformamide and 3 ml of acetonitrile and separated by means of preparative HPLC (acetonitrile/water gradient+0.1% TFA). The product-containing fractions were concentrated and dried under high vacuum. Subsequently, they were dissolved in methanol, admixed with 0.1 ml of 4N hydrogen chloride in dioxane and concentrated. The solid was dried under high vacuum. 46 mg (87% of theory) of the title compound were obtained.

¹H NMR (400 MHz, DMSO-d₆): δ=ppm 0.84-0.99 (m, 2H), 1.10-1.33 (m, 2H), 1.41-1.52 (m, 1H), 1.54-1.61 (m, 1H), 1.74 (m, 3H), 2.04-2.20 (m, 2H), 2.34-2.43 (m, 1H), 2.47 (s, 3H), 2.63 (m, 2H), 2.98 (dd, 1H), 3.16 (dd, 1H), 3.25 (dd, 2H), 4.52 (m, 2H), 4.77 (m, 1H), 7.37 (d, 2H), 7.45 (d, 2H), 7.77 (d, 1H), 7.84 (m, 5H), 7.91-7.95 (m, 2H), 8.03 (d, 2H), 8.30 (d, 1H), 8.83 (d, 1H), 10.58 (s, 1H)

LC-MS (Method 1): Rₜ=0.66 min; MS (ESIpos): m/z=665 [M+H—HCl]⁺.

### Example 27

### N-alpha-{[trans-4-(Aminomethyl)cyclohexyl]carbonyl}-4-(6-{[4-(dimethylamino)cyclohexyl]-carbamoyl}-2-methylpyridin-3-yl)-N-(2-oxo-2,3-dihydro-1H-benzimidazol-5-yl)-L-phenylalaninamide hydrochloride

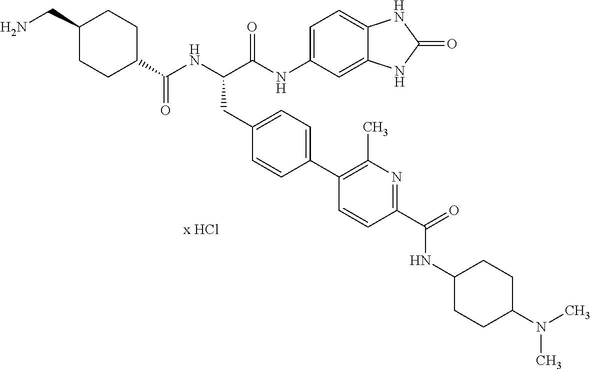

To a solution of 20.8 mg (26 μmol) of N-alpha-[(trans-4-{[(tert-butoxycarbonyl)amino]methyl}-cyclohexyl)carbonyl]-4-(6-{[4-(dimethylamino)cyclohexyl]carbamoyl}-2-methylpyridin-3-yl)-N-(2-oxo-2,3-dihydro-1H-benzimidazol-5-yl)-L-phenylalaninamide trifluoroacetate in 1.5 ml of dioxane were added 89 μl (0.39 mmol) of 4M hydrogen chloride in dioxane. The mixture was stirred at RT overnight. The reaction mixture was concentrated, taken up in 0.5 ml of dimethyl sulphoxide and a little acetonitrile and separated by means of preparative HPLC (acetonitrile/water gradient+0.1% TFA). The product-containing fractions were concentrated and dried under high vacuum. Subsequently, they were dissolved in methanol, admixed with 0.1 ml of 4N hydrogen chloride in dioxane and concentrated. The solid was dried under high vacuum. 5 mg (23% of theory) of the title compound were obtained.

LC-MS (Method 1): Rₜ=0.52 min; MS (ESIpos): m/z=695 [M+H—HCl]⁺.

### Example 28

### N-alpha-{[trans-4-(Aminomethyl)cyclohexyl]carbonyl}-4-(4-methyl-6-{[(3R)-2-oxopiperidin-3-yl]carbamoyl}pyridin-3-yl)-N-(2-oxo-2,3-dihydro-1H-benzimidazol-5-yl)-L-phenylalaninamide hydrochloride

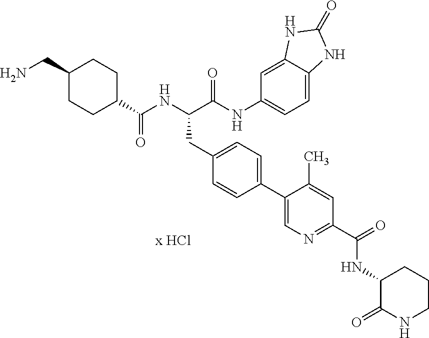

To a solution of 13.2 mg (17.2 μmol) of N-alpha-[(trans-4-{[(tert-butoxycarbonyl)amino]-methyl}cyclohexyl)carbonyl]-4-(4-methyl-6-{[(3R)-2-oxopiperidin-3-yl]carbamoyl}pyridin-3-yl)-N-(2-oxo-2,3-dihydro-1H-benzimidazol-5-yl)-L-phenylalaninamide in 0.5 ml of dichloromethane were added 21.5 μl (86 μmol) of 4M hydrogen chloride in dioxane. The mixture was then stirred at RT for 24 h. The reaction mixture was admixed with acetonitrile. The precipitated product was filtered off with suction, washed with acetonitrile and dried under high vacuum. 10 mg (79% of theory) of the title compound were obtained.

¹H NMR (300 MHz, DMSO-d₆): δ=ppm 0.81-1.00 (m, 2H), 1.06-1.22 (m, 2H), 1.38-1.50 (m, 1H), 1.51-1.60 (m, 1H), 1.65-1.87 (m, 7H), 2.12-2.24 (m, 1H), 2.31 (s, 3H), 2.57-2.66 (m, 2H), 2.91-3.00 (m, 1H), 3.07-3.21 (m, 3H), 4.26-4.38 (m, 1H), 4.64-4.76 (m, 1H), 6.83 (d, 1H), 7.02 (dd, 1H), 7.34 (d, 2H), 7.39-7.45 (m, 3H), 7.65-7.70 (m, 1H), 7.70-7.86 (m, 3H), 7.97 (s, 1H), 8.13-8.18 (m, 1H), 8.38 (s, 1H), 8.82 (d, 1H), 9.96-10.04 (m, 1H), 10.45-10.50 (m, 1H), 10.55 (s, 1H)

LC-MS (Method 4) Rₜ=0.68 min; MS (ESIpos): m/z=667 [M+H—HCl]⁺.

### Example 29

### N-alpha-{[trans-4-(Aminomethyl)cyclohexyl]carbonyl}-4-(6-{[(2R)-1-hydroxypropan-2-yl]carbamoyl}-2-methylpyridin-3-yl)-N-[4-(2H-tetrazol-5-yl)phenyl]-L-phenylalaninamide hydrochloride

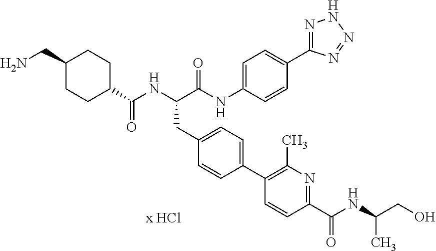

To a solution of 61.9 mg (72 μmol) of N-alpha-[(trans-4-{[(tert-butoxycarbonyl)amino]-methyl}cyclohexyl)carbonyl]-4-(6-{[(2R)-1-hydroxypropan-2-yl]carbamoyl}-2-methylpyridin-3-yl)-N-[4-(2H-tetrazol-5-yl)phenyl]-L-phenylalaninamide trifluoroacetate in 2.5 ml of dioxane was added 0.27 ml (1.09 mmol) of 4M hydrogen chloride in dioxane. The mixture was stirred at RT overnight. The precipitated product was filtered off with suction, washed with acetonitrile and dried under high vacuum. 56 mg (100% of theory) of the title compound were obtained.

¹H NMR (400 MHz, DMSO-d₆): δ=ppm 0.83-0.99 (m, 2H), 1.08-1.15 (m, 1H), 1.18 (d, 3H), 1.23-1.36 (m, 1H), 1.41-1.51 (m, 1H), 1.53-1.60 (m, 1H), 1.69-1.80 (m, 3H), 2.11-2.20 (m, 1H), 2.46 (s, 3H), 2.63 (m, 2H), 2.98 (dd, 1H), 3.16 (dd, 1H), 3.48 (m, 2H), 3.99-4.08 (m, 1H), 4.72-4.81 (m, 1H), 7.35 (d, 2H), 7.44 (d, 2H), 7.76 (d, 1H), 7.84 (m, 5H), 7.93 (d, 1H), 8.03 (d, 2H), 8.30 (d, 1H), 8.34 (d, 1H), 10.59 (s, 1H)

LC-MS (Method 1): Rₜ=0.67 min; MS (ESIpos): m/z=640 [M+H—HCl]⁺.

### Example 30

### N-alpha-{[trans-4-(Aminomethyl)cyclohexyl]carbonyl}-4{-2-methyl-6-[(1-methylpiperidin-4-yl)carbamoyl]pyridin-3-yl}-N-[4-(1H-tetrazol-5-yl)phenyl]-L-phenylalaninamide hydrochloride

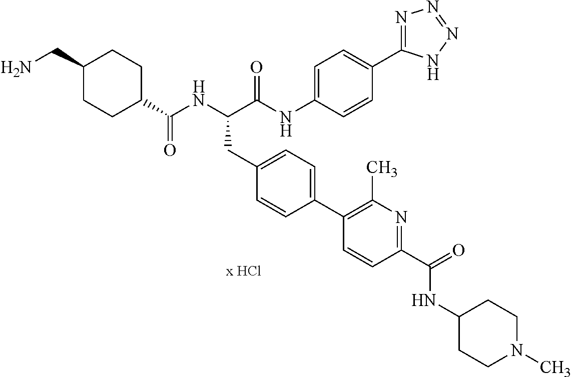

To a solution of 86.7 mg (97 μmol) of N-alpha-[(trans-4-{[(tert-butoxycarbonyl)amino]-methyl}cyclohexyl)carbonyl]-4-{2-methyl-6-[(1-methylpiperidin-4-yl)carbamoyl]pyridin-3-yl}-N-[4-(2H-tetrazol-5-yl)phenyl]-L-phenylalaninamide trifluoroacetate in 3 ml of dioxane were added 364 μl (1.46 mmol) of 4M hydrogen chloride in dioxane. The mixture was then stirred at RT for 24 h. The reaction mixture was admixed with acetonitrile. The precipitated product was filtered off with suction, washed with acetonitrile and dried under high vacuum. 64 mg (84% of theory) of the title compound were obtained.

¹H NMR (400 MHz, DMSO-d₆): δ=ppm 0.82-0.98 (m, 2H), 1.11-1.35 (m, 2H), 1.43-1.53 (m, 1H), 1.55-1.63 (m, 1H), 1.74 (m, 3H), 1.94-2.04 (m, 4H), 2.08 (s, 2H), 2.11-2.20 (m, 1H), 2.46 (s, 2H), 2.59-2.67 (m, 2H), 2.70-2.76 (m, 2H), 2.98 (dd, 1H), 3.04-3.18 (m, 3H), 3.40-3.48 (m, 2H), 3.98-4.09 (m, 1H), 4.72-4.80 (m, 1H), 7.34 (d, 2H), 7.44 (d, 2H), 7.74 (d, 1H), 7.83 (m, 5H), 7.91 (d, 1H), 8.02 (d, 2H), 8.28-8.34 (m, 1H), 8.67 (d, 1H), 10.10-10.41 (m, 1H), 10.53-10.60 (m, 1H)

LC-MS (Method 1): Rₜ=0.55 min; MS (ESIpos): m/z=679 [M+H—HCl]⁺.

### Example 31

### 4-{6-[(trans-4-Aminocyclohexyl)carbamoyl]-2-methylpyridin-3-yl}-N-alpha-{[trans-4-(aminomethyl)cyclohexyl]carbonyl}-N-[4-(2H-tetrazol-5-yl)phenyl]-L-phenylalaninamide hydrochloride

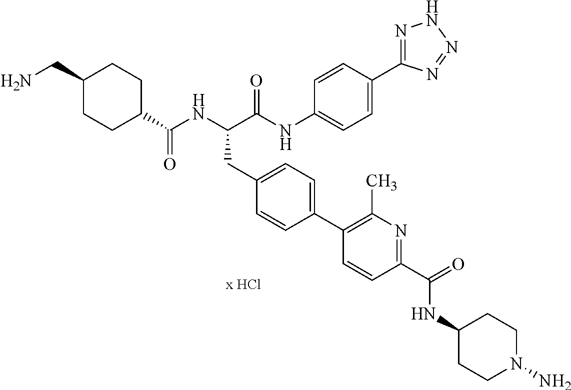

To a solution of 67.3 mg (75 μmol) of 4-{6-[(trans-4-aminocyclohexyl)carbamoyl]-2-methylpyridin-3-yl}-N-alpha-[(trans-4-{[(tert-butoxycarbonyl)amino]methyl}cyclohexyl)-carbonyl]-N-[4-(2H-tetrazol-5-yl)phenyl]-L-phenylalaninamide trifluoroacetate in 2.5 ml of dioxane were added 283 μl (1.13 mmol) of 4M hydrogen chloride in dioxane. The mixture was then stirred at RT for 24 h. The precipitated product was filtered off with suction, washed with acetonitrile and dried under high vacuum. 52 mg (89% of theory) of the title compound were obtained.

¹H NMR (400 MHz, DMSO-d₆): δ=ppm 0.84-0.99 (m, 2H), 1.10-1.34 (m, 2H), 1.41-1.61 (m, 6H), 1.69-1.81 (m, 3H), 1.89 (m, 2H), 2.01 (d, 2H), 2.11-2.20 (m, 1H), 2.45 (s, 3H), 2.63 (m, 2H), 2.97 (dd, 1H), 3.15 (dd, 1H), 3.72-3.83 (m, 1H), 4.76 (m, 2H), 7.34 (d, 2H), 7.44 (d, 2H), 7.74 (d, 1H), 7.84 (d, 2H), 7.87-7.93 (m, 3H), 8.03 (m, 5H), 8.32 (d, 1H), 8.39 (d, 1H), 10.59 (br. s., 1H)

LC-MS (Method 1): Rₜ=0.57 min; MS (ESIpos): m/z=679.3 [M+H—HCl]⁺.

### Example 32

### N-alpha-{[trans-4-(Aminomethyl)cyclohexyl]carbonyl}-4-{4-methyl-6-[(3-oxopiperazin-1-yl)carbonyl]pyridin-3-yl}-N-[4-(1H-tetrazol-5-yl)phenyl]-L-phenylalaninamide hydrochloride

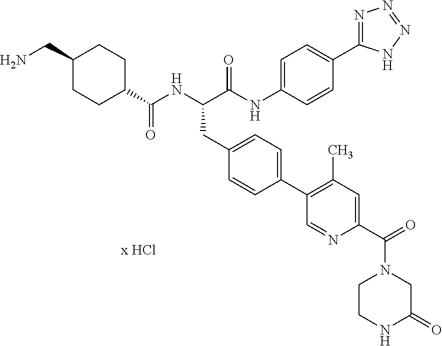

To a solution of 34 mg (44.5 μmol) of N-alpha-[(trans-4-{[(tert-Butoxycarbonyl)amino]-methyl}cyclohexyl)carbonyl]-4-{4-methyl-6-[(3-oxopiperazin-1-yl)carbonyl]pyridin-3-yl}-N-[4-(1H-tetrazol-5-yl)phenyl]-L-phenylalaninamide in 1.3 ml of dichloromethane were added 111 μl (0.36 mmol) of 4M hydrogen chloride in dioxane. The mixture was then stirred at RT for 24 h. The reaction mixture was admixed with acetonitrile. The precipitated product was filtered off with suction, washed with acetonitrile and dried under high vacuum. The residue was purified by preparative HPLC (Method 10). The product-containing fractions were admixed with a little 4M hydrogen chloride in dioxane and lyophilized. 7 mg (23% of theory) of the title compound were obtained.

¹H NMR (300 MHz, DMSO-d₆): δ=ppm 0.81-0.99 (m, 2H), 1.09-1.34 (m, 2H), 1.37-1.49 (m, 1H), 1.52-1.61 (m, 1H), 1.65-1.81 (m, 3H), 2.09-2.20 (m, 1H), 2.28 (s, 4H), 2.60-2.65 (m, 2H), 2.70-2.74 (m, 2H), 2.94 (dd, 1H), 3.10-3.18 (m, 1H), 3.68-3.74 (m, 1H), 3.78-3.84 (m, 1H), 4.14 (s, 2H), 4.69-4.81 (m, 1H), 7.32-7.39 (m, 2H), 7.40-7.46 (m, 2H), 7.55-7.63 (m, 4H), 7.89 (d, 3H), 8.08-8.19 (m, 2H), 8.33-8.38 (m, 1H), 10.06-10.14 (m, 1H)

LC-MS (Method 4) Rₜ=0.66 min; MS (ESIpos): m/z=665 [M+H—HCl]⁺.

### Example 33

### N-alpha-{[trans-4-(Aminomethyl)cyclohexyl]carbonyl}-4-{4-methyl-6-[(4-methylpiperazin-1-yl)carbonyl]pyridin-3-yl}-N-[4-(1H-tetrazol-5-yl)phenyl]-L-phenylalaninamide hydrochloride

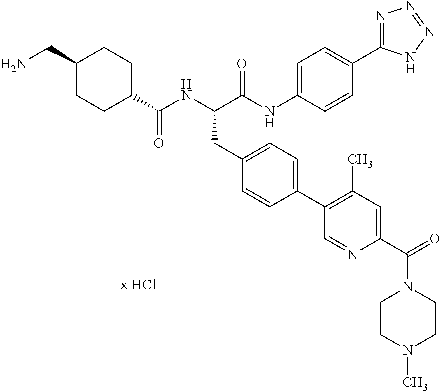

To a solution of 25 mg (32.7 μmol) of N-alpha-[(trans-4-{[(tert-Butoxycarbonyl)amino]-methyl}cyclohexyl)carbonyl]-4-{4-methyl-6-[(4-methylpiperazin-1-yl)carbonyl]pyridin-3-yl}-N-[4-(1H-tetrazol-5-yl)phenyl]-L-phenylalaninamide in 1.3 ml of dichloromethane were added 82 μl (0.33 mmol) of 4M hydrogen chloride in dioxane. The mixture was then stirred at RT for 24 h. The reaction mixture was admixed with acetonitrile. The precipitated product was filtered off with suction, washed with acetonitrile and dried under high vacuum. 18 mg (73% of theory) of the title compound were obtained.

¹H NMR (300 MHz, DMSO-d₆): δ=ppm 0.81-0.99 (m, 2H), 1.09-1.32 (m, 2H), 1.40-1.52 (m, 1H), 1.53-1.62 (m, 1H), 1.65-1.81 (m, 3H), 2.08-2.20 (m, 1H), 2.27 (s, 3H), 2.58-2.65 (m, 2H), 2.79 (br. s., 3H), 2.97 (dd, 1H), 3.04-3.19 (m, 3H), 3.21-3.29 (m, 1H), 3.31-3.42 (m, 1H), 3.46-3.60 (m, 2H), 4.13-4.22 (m, 1H), 4.53-4.63 (m, 1H), 4.69-4.81 (m, 1H), 7.34 (d, 2H), 7.43 (d, 2H), 7.61 (s, 1H), 7.82 (m, 5H), 8.01 (d, 2H), 8.25-8.38 (m, 2H), 10.58 (s, 1H), 10.98 (br. s, 1H)

LC-MS (Method 4) Rₜ=0.58 min; MS (ESIpos): m/z=665.4 [M+H—HCl]⁺.

### Example 34

### N-alpha-{[trans-4-(Aminomethyl)cyclohexyl]carbonyl}-4-[6-(cyclobutylcarbamoyl)-2-methylpyridin-3-yl]-N-1H-indazol-6-yl-L-phenylalaninamide hydrochloride

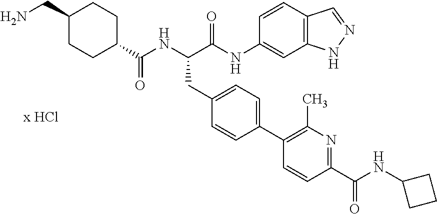 [SMILES: Cc1nc(C(=O)NC2CCC2)ccc1-c1ccc(C[C@H](NC(=O)[C@H]2CC[C@H](CN)CC2)C(=O)Nc2ccc3cn[nH]c3c2)cc1]

A solution of 76 mg (0.1 mmol) of N-alpha-[(trans-4-{[(tert-butoxycarbonyl)amino]methyl}-cyclohexyl)carbonyl]-4-[6-(cyclobutylcarbamoyl)-2-methylpyridin-3-yl]-N-1H-indazol-6-yl-L-phenylalaninamide in 10 ml of dichloromethane was admixed with 0.18 ml (0.75 mmol) of 4M hydrogen chloride in 1,4-dioxane and stirred at RT overnight. The reaction mixture was admixed with acetonitrile, and the precipitate was filtered off with suction and dried under reduced pressure. 59 mg (82% of theory) of the title compound were obtained.

¹H NMR (400 MHz, DMSO-d₆): δ=ppm 0.82-1.00 (m, 2H), 1.09-1.33 (m, 2H), 1.40-1.51 (m, 1H), 1.53-1.61 (m, 1H), 1.63-1.82 (m, 5H), 2.09-2.26 (m, 5H), 2.58-2.64 (m, 2H), 2.97 (dd, 1H), 3.16 (dd, 1H), 4.39-4.51 (m, 1H), 4.71-4.82 (m, 1H), 7.11-7.16 (m, 1H), 7.33 (d, 2H), 7.42 (d, 2H), 7.65 (d, 1H), 7.73 (d, 1H), 7.79-7.91 (m, 4H), 7.96 (d, 1H), 8.11 (s, 1H), 8.22-8.28 (m, 1H), 8.70-8.76 (m, 1H), 10.31-10.36 (m, 1H).

LC-MS (Method 4): Rₜ=0.90 min; MS (ESIpos): m/z=608.5 [M+H—HCl]⁺.

### Example 35

### N-alpha-{[trans-4-(Aminomethyl)cyclohexyl]carbonyl}-N-1H-indazol-6-yl-4-{2-methyl-6-[(1,1,1-trifluoropropan-2-yl)carbamoyl]pyridin-3-yl}-L-phenylalaninamide hydrochloride

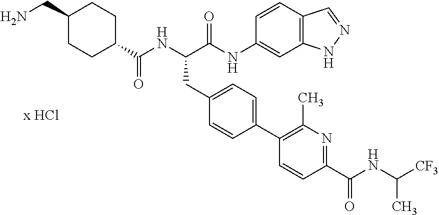 [SMILES: Cc1nc(C(=O)NC(C)C(F)(F)F)ccc1-c1ccc(C[C@H](NC(=O)[C@H]2CC[C@H](CN)CC2)C(=O)Nc2ccc3cn[nH]c3c2)cc1]

A solution of 88 mg (0.12 mmol) of N-alpha-[(trans-4-{[(tert-butoxycarbonyl)amino]-methyl}cyclohexyl)carbonyl]-N-1H-indazol-6-yl-4-{2-methyl-6-[(1,1,1-trifluoropropan-2-yl)-carbamoyl]pyridin-3-yl}-L-phenylalaninamide in 10 ml of dichloromethane was admixed with 0.2 ml (0.82 mmol) of 4M hydrogen chloride in 1,4-dioxane and stirred at RT overnight. The reaction mixture was admixed with acetonitrile, and the precipitate was filtered off with suction and dried under reduced pressure. 69 mg (82% of theory) of the title compound were obtained.

¹H NMR (400 MHz, DMSO-d₆): δ=ppm 0.84-1.00 (m, 2H), 1.10-1.35 (m, 2H), 1.44 (d, 3H), 1.47-1.52 (m, 1H), 1.53-1.63 (m, 1H), 1.69-1.82 (m, 3H), 2.10-2.20 (m, 1H), 2.62 (m, 2H), 2.98 (dd, 1H), 3.17 (dd, 1H), 4.72-4.82 (m, 1H), 4.84-4.94 (m, 1H), 7.14-7.19 (m, 1H), 7.36 (d, 2H), 7.45 (d, 2H), 7.67 (d, 1H), 7.77 (d, 1H), 7.88 (br. s., 3H), 7.95 (d, 1H), 7.98 (s, 1H), 8.14 (s, 1H), 8.26-8.32 (m, 1H), 8.92 (d, 1H), 10.37 (s, 1H).

LC-MS (Method 4): Rₜ=0.94 min; MS (ESIpos): m/z=650.5 [M+H—HCl⁻]⁺.

### Example 36

### N-alpha-{[trans-4-(Aminomethyl)cyclohexyl]carbonyl}-4-{2-methyl-6-[(1,1,1-trifluoropropan-2-yl)carbamoyl]pyridin-3-yl}-N-(2-oxo-2,3-dihydro-1H-benzimidazol-5-yl)-L-phenylalaninamide hydrochloride

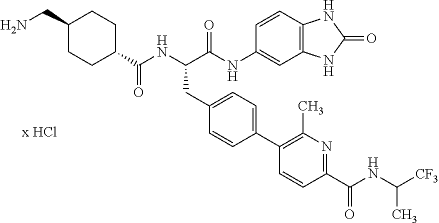 [SMILES: Cc1nc(C(=O)NC(C)C(F)(F)F)ccc1-c1ccc(C[C@H](NC(=O)[C@H]2CC[C@H](CN)CC2)C(=O)Nc2ccc3[nH]c(=O)[nH]c3c2)cc1]

A solution of 58 mg (0.07 mmol) of N-alpha-[(trans-4-{[(tert-butoxycarbonyl)amino]methyl}-cyclohexyl)carbonyl]-4-{2-methyl-6-[(1,1,1-trifluoropropan-2-yl)carbamoyl]pyridin-3-yl}-N-(2-oxo-2,3-dihydro-1H-benzimidazol-5-yl)-L-phenylalaninamide in 6 ml of dichloromethane was admixed with 0.1 ml (0.38 mmol) of 4M hydrogen chloride in 1,4-dioxane and stirred at 40° C. for 2 h. The reaction mixture was admixed with acetonitrile, and the precipitate was filtered off with suction and purified by chromatography via HPLC (Method 8). 24 mg (45% of theory) of the title compound were obtained.

¹H NMR (400 MHz, DMSO-d₆): δ=ppm 0.79-1.01 (m, 2H), 1.12-1.37 (m, 2H), 1.39-1.50 (m, 4H), 1.51-1.62 (m, 1H), 1.65-1.83 (m, 3H), 2.10-2.21 (m, 1H), 2.62-2.66 (m, 2H), 2.89-3.00 (m, 1H), 3.07-3.15 (m, 1H), 4.64-4.78 (m, 1H), 4.80-4.95 (m, 1H), 6.78-6.92 (m, 1H), 6.96-7.09 (m, 1H), 7.31-7.38 (m, 2H), 7.38-7.45 (m, 3H), 7.63-7.74 (m, 3H), 7.74-7.80 (m, 1H), 7.90-7.98 (m, 1H), 8.13-8.22 (m, 1H), 8.82-8.97 (m, 1H), 9.91-10.05 (m, 1H), 10.46-10.53 (m, 1H), 10.54-10.62 (m, 1H).

LC-MS (Method 4): Rₜ=0.87 min; MS (ESIpos): m/z=666.6 [M+H—HCl⁻]⁺.

### Example 37

### N-alpha-{[trans-4-(Aminomethyl)cyclohexyl]carbonyl}-4-[6-(cyclobutylcarbamoyl)-2-methylpyridin-3-yl]-N-(2-oxo-2,3-dihydro-1H-benzimidazol-5-yl)-L-phenylalaninamide hydrochloride

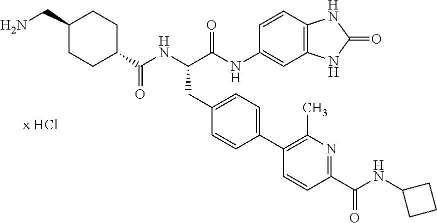 [SMILES: Cc1nc(C(=O)NC2CCC2)ccc1-c1ccc(C[C@H](NC(=O)[C@H]2CC[C@H](CN)CC2)C(=O)Nc2ccc3[nH]c(=O)[nH]c3c2)cc1]

A solution of 83 mg (0.11 mmol) of N-alpha-[(trans-4-{[(tert-butoxycarbonyl)amino]methyl}-cyclohexyl)carbonyl]-4-[6-(cyclobutylcarbamoyl)-2-methylpyridin-3-yl]-N-(2-oxo-2,3-dihydro-1H-benzimidazol-5-yl)-L-phenylalaninamide in 3 ml of dichloromethane was admixed with 0.14 ml (0.57 mmol) of 4M hydrogen chloride in 1,4-dioxane and stirred at 40° C. for 2 h. The reaction mixture was admixed with acetonitrile, and the precipitate was filtered off with suction and purified by chromatography via HPLC (Method 8). 17 mg (22% of theory) of the title compound were obtained.

¹H NMR (400 MHz, DMSO-d₆): δ=ppm 0.81-1.00 (m, 2H), 1.09-1.33 (m, 2H), 1.40-1.51 (m, 1H), 1.53-1.61 (m, 1H), 1.65-1.84 (m, 5H), 2.10-2.28 (m, 5H), 2.62-2.66 (m, 2H), 2.89-2.98 (m, 1H), 3.06-3.13 (m, 1H), 4.39-4.53 (m, 1H), 4.63-4.77 (m, 1H), 6.80-6.90 (m, 1H), 6.98-7.07 (m, 1H), 7.31-7.38 (m, 2H), 7.38-7.46 (m, 3H), 7.66-7.78 (m, 4H), 7.86-7.92 (m, 1H), 8.11-8.21 (m, 1H), 8.69-8.78 (m, 1H), 9.95-10.05 (m, 1H), 10.47-10.53 (m, 1H), 10.55-10.59 (m, 1H).

LC-MS (Method 4): Rₜ=0.83 min; MS (ESIpos): m/z=624.6 [M+H—HCl⁻]⁺.

### Example 38

### N-alpha-{[trans-4-(Aminomethyl)cyclohexyl]carbonyl}-N-(7-chloro-2-oxo-2,3-dihydro-1,3-benzoxazol-5-yl)-4-[6-(isopropylcarbamoyl)-2-methylpyridin-3-yl]-L-phenylalaninamide hydrochloride

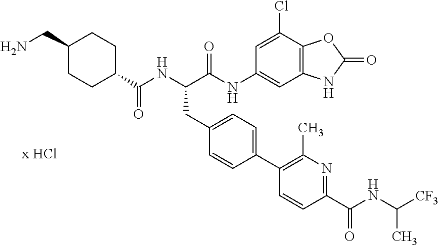 [SMILES: Cc1nc(C(=O)NC(C)C(F)(F)F)ccc1-c1ccc(C[C@H](NC(=O)[C@H]2CC[C@H](CN)CC2)C(=O)Nc2cc(Cl)c3oc(=O)[nH]c3c2)cc1]

A solution of N-alpha-[(trans-4-{[(tert-butoxycarbonyl)amino]methyl}cyclohexyl)carbonyl]-N-(7-chloro-2-oxo-2,3-dihydro-1,3-benzoxazol-5-yl)-4-[6-(isopropylcarbamoyl)-2-methylpyridin-3-yl]-L-phenylalaninamide (40 mg, 0.054 mmol) in dioxane (2 ml) was admixed with 4M hydrogen chloride in 1,4-dioxane (0.20 ml, 0.80 mmol) and stirred at RT overnight. The solid was filtered off with suction, washed with dioxane and acetonitrile, then dried under high vacuum. This gave 35 mg (96% of theory) of the title compound.

¹H NMR (400 MHz, DMSO-d₆): δ=ppm 0.81-1.02 (m, 2H), 1.23 (m, 8H), 1.41-1.52 (m, 1H), 1.53-1.65 (m, 1H), 1.67-1.83 (m, 3H), 2.07-2.22 (m, 1H), 2.45 (s, 3H), 2.59-2.70 (m, 2H), 2.90-3.00 (m, 1H), 3.11 (m, 1H), 4.05-4.21 (m, 1H), 4.62-4.75 (m, 1H), 7.30-7.36 (m, 2H), 7.37-7.45 (m, 4H), 7.75 (m, 4H), 7.88-7.94 (m, 1H), 8.24 (d, 1H), 8.30 (d, 1H), 10.35 (s, 1H), 11.94 (s, 1H).

LC-MS (Method 1): Rₜ=0.80 min; MS (ESIpos): m/z=647 [M+H—HCl⁻]⁺.

### Example 39

### N-alpha-{[trans-4-(Aminomethyl)cyclohexyl]carbonyl}-4-[6-(isopropylcarbamoyl)-2-methylpyridin-3-yl]-N-(3-oxo-2,3-dihydro-1H-indazol-6-yl)-L-phenylalaninamide hydrochloride

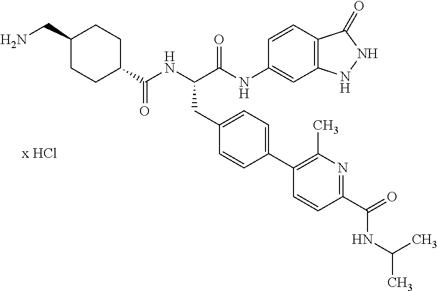 [SMILES: Cc1nc(C(=O)NC(C)C)ccc1-c1ccc(C[C@H](NC(=O)[C@H]2CC[C@H](CN)CC2)C(=O)Nc2ccc3c(=O)[nH][nH]c3c2)cc1]

A solution of N-alpha-[(trans-4-{[(tert-butoxycarbonyl)amino]methyl}cyclohexyl)carbonyl]-4-[6-(isopropylcarbamoyl)-2-methylpyridin-3-yl]-N-(3-oxo-2,3-dihydro-1H-indazol-6-yl)-L-phenylalaninamide (74 mg, 0.09 mmol) in dioxane (2 ml) was admixed with 4M hydrogen chloride in 1,4-dioxane (0.34 ml, 1.36 mmol) and stirred at RT overnight. The solvent was removed on a rotary evaporator. The residue was dissolved in a little DMSO, filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 51 mg (87% of theory) of the title compound.

¹H NMR (400 MHz, DMSO-d₆): δ=ppm 0.78-1.01 (m, 2H), 1.21 (m, 8H), 1.40-1.61 (m, 2H), 1.64-1.83 (m, 2H), 2.08-2.21 (m, 1H), 2.46 (s, 3H), 2.63 (m, 2H), 2.91-3.02 (m, 1H), 3.07-3.21 (m, 1H), 4.05-4.21 (m, 1H), 4.68-4.84 (m, 1H), 7.00-7.08 (m, 1H), 7.34 (d, 2H), 7.40-7.46 (m, 2H), 7.57 (d, 1H), 7.75 (d, 1H), 7.79-7.95 (m, 6H), 8.28 (d, 1H), 8.34 (d, 1H), 10.37 (s, 1H), 11.38 (br. s, 1H)

LC-MS (Method 1): Rₜ=0.66 min; MS (ESIneg): m/z=610 [M−HCl]⁻.

### Example 40

### N-alpha-{[trans-4-(Aminomethyl)cyclohexyl]carbonyl}-4-[6-(isopropylcarbamoyl)-2-methylpyridin-3-yl]-N-(2-oxo-2,3-dihydro-1H-benzimidazol-5-yl)-L-phenylalaninamide hydrochloride

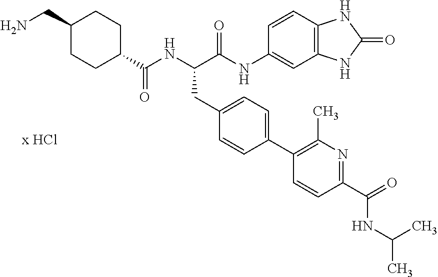 [SMILES: Cc1nc(C(=O)NC(C)C)ccc1-c1ccc(C[C@H](NC(=O)[C@H]2CC[C@H](CN)CC2)C(=O)Nc2ccc3[nH]c(=O)[nH]c3c2)cc1]

A solution of N-alpha-[(trans-4-{[(tert-butoxycarbonyl)amino]methyl}cyclohexyl)carbonyl]-4-[6-(isopropylcarbamoyl)-2-methylpyridin-3-yl]-N-(2-oxo-2,3-dihydro-1H-benzimidazol-5-yl)-L-phenylalaninamide (44.8 mg, 0.07 mmol) in dioxane (1 ml) was admixed with 4M hydrogen chloride in 1,4-dioxane (78 al, 0.32 mmol) and stirred at RT for 3 days. Thereafter, the reaction solution was admixed once again with 4M hydrogen chloride in 1,4-dioxane (78 al, 0.32 mmol) and stirred at 45° C. for 4 days. The solvent was removed on a rotary evaporator and the residue was dissolved in DMSO/acetonitrile (about 2 ml). The solution was filtered through a Millipore filter and purified by preparative HPLC (eluent: gradient of acetonitrile/water with 0.1% trifluoroacetic acid). The substance obtained was taken up in methanol and 4M hydrogen chloride in 1,4-dioxane (about 0.05 ml) was added thereto. The solvent was removed on a rotary evaporator and the residue was dried under high vacuum. This gave 13 mg (12% of theory) of the title compound.

¹H NMR (400 MHz, DMSO-d₆): δ=ppm 0.94 (m, 2H), 1.09-1.33 (m, 10H), 1.42-1.51 (m, 1H), 1.52-1.61 (m, 1H), 1.75 (m, 3H), 2.09-2.20 (m, 1H), 2.47 (s, 3H), 2.62 (m, 2H), 2.95 (dd, 1H), 3.12 (dd, 1H), 4.13 (m, 1H), 4.65-4.78 (m, 1H), 6.84 (d, 1H), 7.05 (dd, 1H), 7.31-7.36 (m, 2H), 7.38-7.45 (m, 3H), 7.75 (d, 1H), 7.81-8.01 (m, 4H), 8.20 (d, 1H), 8.33 (d, 1H), 10.05 (s, 1H), 10.50 (s, 1H), 10.56 (s, 1H).

LC-MS (Method 1): Rₜ=0.67 min; MS (ESIneg): m/z=610 [M−HCl]⁻.

### Example 41

### N-alpha-{[trans-4-(Aminomethyl)cyclohexyl]carbonyl}-4-(4-methyl-6-{[(3R)-2-oxopiperidin-3-yl]carbamoyl}pyridin-3-yl)-N-(3-oxo-2,3-dihydro-1H-indazol-6-yl)-L-phenylalaninamide hydrochloride

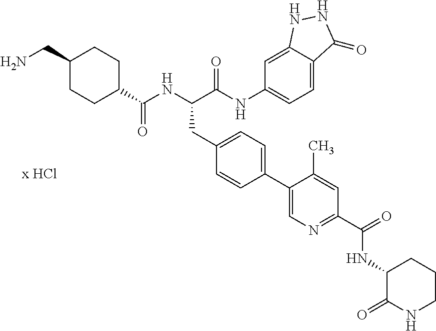

To a solution of N-alpha-[(trans-4-{[(tert-butoxycarbonyl)amino]-methyl}cyclohexyl)-carbonyl]-4-(4-methyl-6-{[(3R)-2-oxopiperidin-3-yl]carbamoyl}pyridin-3-yl)-N-(3-oxo-2,3-dihydro-1H-indazol-6-yl)-L-phenylalaninamide in dichloromethane is added 4M hydrogen chloride in dioxane. The mixture is stirred at RT overnight. The reaction mixture is worked up by methods known to those skilled in the art and the residue is separated by means of preparative HPLC. The title compound is obtained.

### B) Assessment of Physiological Efficacy

The suitability of the inventive compounds for treatment of thromboembolic or hyperfibrinolytic disorders can be demonstrated in the following assay systems:

### a) Test Descriptions (In Vitro)

### a.1) Measurement of FXIa Inhibition

To determine the factor XIa inhibition of the substances according to the invention, a biochemical test system is used which utilizes the reaction of a peptidic factor XIa substrate to determine the enzymatic activity of human factor XIa. Here, factor XIa cleaves from the peptic factor XIa substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions are placed into the wells of white microtitre plates from Greiner (384 wells). Subsequently, the following are added successively: 20 μl of assay buffer (50 mmol/1 Tris buffer pH 7.4; 100 mmol/1 sodium chloride; 5 mmol/1 calcium chloride; 0.1% bovine serum albumin) and 20 μl of factor XIa from Kordia (0.45 nM in assay buffer). After 15 min of incubation, the enzyme reaction is started by addition of 20 μl of the factor XIa substrate Boc-Glu(OBzl)-Ala-Arg-AMC dissolved in assay buffer (10 μM in assay buffer) from Bachem, the mixture is incubated at room temperature (22° C.) for 30 min and fluorescence is then measured (excitation: 360 nm, emission: 460 nm). The measured emissions of the test batches with test substance are compared to those of control batches without test substance (only dimethyl sulphoxide instead of test substance in dimethyl sulphoxide), and the IC₅₀ values are calculated from the concentration/activity relationships. Activity data from this test are listed in Table A below:

TABLE A

| Example No. | IC₅₀ [nM] | Example No. | IC₅₀ [nM] |
|---|---|---|---|
| 1 | 2.0 | 2 | 1.9 |
| 3 | 5.6 | 4 | 1.3 |
| 5 | 1.9 | 6 | 4.1 |
| 7 | 2.6 | 8 | 2.2 |
| 9 | 5.2 | 10 | 3.0 |
| 11 | 5.2 | 12 | 4.8 |
| 13 | 0.9 | 14 | 30 |
| 15 | 3.2 | 16 | 8.1 |
| 17 | 5.0 | 18 | 4.5 |
| 19 | 4.5 | 20 | 3.9 |
| 21 | 1.3 | 22 | 2.5 |
| 23 | 2.5 | 24 | 6.0 |
| 25 | 4.2 | 26 | 1.3 |
| 27 | 19 | 28 | 20 |
| 29 | 2.4 | 30 | 5.5 |
| 31 | 1.9 | 32 | 1.8 |
| 33 | 4.2 | 34 | 8.2 |
| 35 | 8.7 | 36 | 11 |
| 37 | 13 | 38 | 14 |
| 39 | 4.3 | 40 | 15.5 |

### a.2) Determination of the Selectivity

To demonstrate the selectivity of the substances with respect to FXIa inhibition, the test substances are examined for their inhibition of other human serin proteases, such as factor Xa, trypsin and plasmin. To determine the enzymatic activity of factor Xa (1.3 nmol/1 from Kordia), trypsin (83 mU/ml from Sigma) and plasmin (0.1 μg/ml from Kordia), these enzymes are dissolved (50 mmol/1 of Tris buffer [C,C,C-tris(hydroxymethyl)aminomethane], 100 mmol/1 of sodium chloride, 0.1% BSA [bovine serum albumin], 5 mmol/1 of calcium chloride, pH 7.4) and incubated for 15 min with test substance in various concentrations in dimethyl sulphoxide and also with dimethyl sulphoxide without test substance. The enzymatic reaction is then started by addition of the appropriate substrates (5 μmol of Boc-Ile-Glu-Gly-Arg-AMC from Bachem for factor Xa and trypsin, 50 μmol of MeOSuc-Ala-Phe-Lys-AMC from Bachem for plasmin). After an incubation time of 30 min at 22° C., fluorescence is measured (excitation: 360 nm, emission: 460 nm). The measured emissions of the test batches with test substance are compared to the control batches without test substance (only dimethyl sulphoxide instead of test substance in dimethyl sulphoxide) and the IC₅₀ values are calculated from the concentration/activity relationships.

### a.3) Thrombin Generation Assay (Thrombogram)

The effect of the test substances on the thrombogram (thrombin generation assay according to Hemker) is determined in vitro in human plasma (Octaplas® from Octapharma).

In the thrombin generation assay according to Hemker, the activity of thrombin in coagulating plasma is determined by measuring the fluorescent cleavage products of the substrate I-1140 (Z-Gly-Gly-Arg-AMC, Bachem). The reactions are carried out in the presence of varying concentrations of test substance or the corresponding solvent. To start the reaction, reagents from Thrombinoscope (30 pM or 0.1 pM recombinant tissue factor, 24 μM phospholipids in HEPES) are used. Moreover, a thrombin calibrator from Thrombinoscope is used whose amidolytic activity is required for calculating the thrombin activity in a sample containing an unknown amount of thrombin. The test is carried out according to the specifications of the manufacturer (Thrombinoscope BV): 4 μl of test substance or of the solvent, 76 μl of plasma and 20 μl of PPP reagent or thrombin calibrator are incubated at 37° C. for 5 min. After addition of 20 μl of 2.5 mM thrombin substrate in 20 mM HEPES, 60 mg/ml of BSA, 102 mM of calcium chloride, the thrombin generation is measured every 20 s over a period of 120 min. Measurement is carried out using a fluorometer (Fluoroskan Ascent) from Thermo Electron fitted with a 390/460 nM filter pair and a dispenser.

Using the Thrombinoscope software, the thrombogram is calculated and represented graphically. The following parameters are calculated: lag time, time to peak, peak, ETP (endogenous thrombin potential) and start tail.

### a.4) Determination of the Anticoagulatory Activity

The anticoagulatory activity of the test substances is determined in vitro in human and animal plasma (for example mouse, rat, rabbit, pig and dog plasma). To this end, blood is drawn off in a mixing ratio of sodium citrate/blood of 1:9 using a 0.11 molar sodium citrate solution as receiver. Immediately after the blood has been drawn off, it is mixed thoroughly and centrifuged at about 4000 g for 15 minutes. The supernatant is pipetted off.

The prothrombin time (PT, synonyms: thromboplastin time, quick test) is determined in the presence of varying concentrations of test substance or the corresponding solvent using a commercial test kit (Neoplastin® from Boehringer Mannheim or Hemoliance® RecombiPlastin from Instrumentation Laboratory). The test compounds are incubated with the plasma at 37° C. for 3 minutes. Coagulation is then started by addition of thromboplastin, and the time when coagulation occurs is determined. The concentration of test substance which effects a doubling of the prothrombin time is determined.

The activated partial thromboplastin time (aPTT) is determined in the presence of varying concentrations of test substance or the corresponding solvent using a commercial test kit (C.K. Prest from Diagnostica Stago). The test compounds are incubated with the plasma and the PTT reagent (cephalin, kaolin) at 37° C. for 3 minutes. Coagulation is then started by addition of 25 mM aqueous calcium chloride, and the time when coagulation occurs is determined. The concentration of test substance which brings about a 1.5-fold extension of the aPTT is determined. Activity data from this test are listed in Table B below:

TABLE B

| Example No. | aPTT [μmol/l] | Example No. | aPTT [μmol/l] |
|---|---|---|---|
| 1 | 0.1 | 2 | 0.08 |
| 3 | 0.16 | 4 | 0.2 |
| 5 | 0.04 | 6 | 0.04 |
| 7 | 0.04 | 8 | 0.05 |
| 9 | 0.05 | 10 | 0.07 |
| 11 | 0.07 | 12 | 0.08 |
| 13 | 0.08 | 14 | 0.08 |
| 15 | 0.1 | 16 | 0.11 |
| 17 | 0.12 | 18 | 0.12 |
| 19 | 0.12 | 20 | 0.13 |
| 21 | 0.15 | 22 | 0.16 |
| 23 | 0.17 | 24 | 0.17 |
| 25 | 0.18 | 26 | 0.22 |
| 27 | 0.22 | 28 | 0.39 |
| 33 | 0.44 | 34 | 0.41 |
| 35 | 0.49 | 36 | 0.21 |
| 37 | 0.2 | 38 | 0.99 |
| 39 | 0.27 | | |

### a.5) Determination of Fibrinolytic Activity

Antifibrinolytic activity in vitro is assessed in human, platelet-free plasma. Tissue factor (TF) (1 pM) and tissue plasminogen activator (tPA) (40 nM) are pipetted into plasma together with 12.5 mM aqueous calcium chloride solution and substance. On occurrence of clotting, the subsequent clot lysis is determined photometrically over a period of 30 minutes.

### a.6) Measurement of Plasmin Inhibition

The plasmin inhibition of the inventive substances is determined using a biochemical test system which utilizes the reaction of a peptidic plasmin substrate to determine the enzymatic activity of human plasmin. Here, plasmin cleaves from the peptic plasmin substrate the C-terminal aminomethylcoumarin (AMC), the fluorescence of which is measured. The determinations are carried out in microtitre plates.

Test substances are dissolved in dimethyl sulphoxide and serially diluted in dimethyl sulphoxide (3000 μM to 0.0078 μM; resulting final concentrations in the test: 50 μM to 0.00013 μM). In each case 1 μl of the diluted substance solutions are placed into the wells of white microtitre plates from Greiner (384 wells). Subsequently, the following are added successively: 20 μl of assay buffer (50 mmol/1 Tris buffer pH 7.4; 100 mmol/1 sodium chloride; 5 mmol/1 calcium chloride; 0.1% bovine serum albumin) and 20 μl of plasmin from Kordia (0.3 μg/ml in assay buffer). After 15 min of incubation, the enzyme reaction is started by addition of 20 μl of the plasmin substrate MeOSuc-Ala-Phe-Lys-AMC dissolved in assay buffer (150 μM in assay buffer) from Bachem, the mixture is incubated at room temperature (22° C.) for 30 min and fluorescence is then measured (excitation: 360 nM, emission: 460 nM). The measured emissions of the test batches with test substance are compared to those of control batches without test substance (only dimethyl sulphoxide instead of test substance in dimethyl sulphoxide), and the IC₅₀ values are calculated from the concentration/activity relationships. Activity data from this test are listed in Table C below:

TABLE C

| Example No. | IC₅₀ [nM] | Example No. | IC₅₀ [nM] |
|---|---|---|---|
| 23 | 4.5 | 29 | 10 |
| 34 | 0.9 | 35 | 1.1 |
| 36 | 1.2 | 37 | 1.5 |
| 38 | 0.8 | 39 | 1.2 |
| 40 | 1.8 | | |

### b) Determination of Antithrombotic Activity (In Vivo)

b.1) Arterial Thrombosis Model (Iron(II) Chloride-Induced Thrombosis) in Combination with Ear Bleeding Time in Rabbits

The antithrombotic activity of the FXIa inhibitors is tested in an arterial thrombosis model. Thrombus formation is triggered here by causing chemical injury to a region in the carotid artery in rabbits. Simultaneously, the ear bleeding time is determined.

Male rabbits (Crl:KBL (NZW)BR, Charles River) receiving a normal diet and having a body weight of 2.2-2.5 kg are anaesthetized by intramuscular administration of xylazine and ketamine (Rompun, Bayer, 5 mg/kg and Ketavet, Pharmacia & Upjohn GmbH, 40 mg/kg body weight). Anaesthesia is furthermore maintained by intravenous administration of the same preparations (bolus: continuous infusion) via the right auricular vein.

The right carotid artery is exposed and the tissue injury is then caused by wrapping a piece of filter paper (10 mm×10 mm) on a Parafilm® strip (25 mm×12 mm) around the carotid artery without disturbing the blood flow. The filter paper contains 100 μl of a 13% strength solution of iron(II) chloride (Sigma) in water. After 5 min, the filter paper is removed and the vessel is rinsed twice with aqueous 0.9% strength sodium chloride solution. 30 min after the injury the injured region of the carotid artery is extracted surgically and any thrombotic material is removed and weighed.

The test substances are administered either intravenously to the anaesthetized animals via the femoral vein or orally to the awake animals via gavage, in each case 5 min and 2 h, respectively, before the injury.

Ear bleeding time is determined 2 min after injury to the carotid artery. To this end, the left ear is shaved and a defined 3-mm-long incision (blade Art. Number 10-150-10, Martin, Tuttlingen, Germany) is made parallel to the longitudinal axis of the ear. Care is taken here not to damage any visible vessels. Any blood that extravasates is taken up in 15 second intervals using accurately weighed filter paper pieces, without touching the wound directly. Bleeding time is calculated as the time from making the incision to the point in time where no more blood can be detected on the filter paper. The volume of the extravasated blood is calculated after weighing of the filter paper pieces.

### c) Determination of Fibrinolytic Activity (In Vivo)

### c.1) Hyper-Fibrinolytic Rats

The determination of antifibrinolytic activity in vivo is conducted in hyperfibrinolytic rats. After anaesthetization and catheterization of the animals, hyperfibrinolysis is triggered by infusion of tissue plasminogen activator (tPA) (8 mg/kg/h). 10 minutes after commencement of tPA infusion, the substances are administered as an i.v. bolus. After a further 15 minutes, tPA infusion is ended and a transsection of the tail is conducted. Subaqual bleeding (in physiological saline at 37° C.) is observed over 30 minutes and the bleed time is determined.

### C) Working Examples of Pharmaceutical Formulations

The inventive substances can be converted to pharmaceutical formulations, for example, as follows:

### Tablet:

### Composition:

100 mg of the compound of Example 1, 50 mg of lactose (monohydrate), 50 mg of maize starch, 10 mg of polyvinylpyrrolidone (PVP) and 2 mg of magnesium stearate.

Tablet weight 212 mg, diameter 8 mm, radius of curvature 12 mm.

### Production:

The mixture of the compound of Example 1, lactose and starch is granulated with a 5% strength solution (m/m) of the PVP in water. After drying, the granules are mixed with the magnesium stearate for 5 min. This mixture is compressed in a conventional tablet press (see above for format of the tablet).

### Oral Suspension:

### Composition:

1000 mg of the compound of Example 1, 1000 mg of ethanol (96%), 400 mg of Rhodigel and 99 g of water.

A single dose of 100 mg of the compound according to the invention corresponds to 10 ml of oral suspension.

### Production:

The Rhodigel is suspended in ethanol, and the compound of Example 1 is added to the suspension. The water is added while stirring. The mixture is stirred for about 6 h until swelling of the Rhodigel is complete.

### Solution for Oral Administration:

### Composition:

500 mg of the inventive compound, 2.5 g of polysorbate and 97 g of polyethylene glycol 400. A single dose of 100 mg of the inventive compound corresponds to 20 g of oral solution.

### Production:

The compound according to the invention is suspended in the mixture of polyethylene glycol and polysorbate while stirring. The stirring operation is continued until dissolution of the compound according to the invention is complete.

i.v. Solution:

The inventive compound is dissolved in a concentration below the saturation solubility in a physiologically acceptable solvent (e.g. isotonic saline, glucose solution 5% and/or polyethylene glycol 400/water 30% m/m). The solution is subjected to sterile filtration and dispensed into sterile and pyrogen-free injection vessels.

1. A compound of the formula 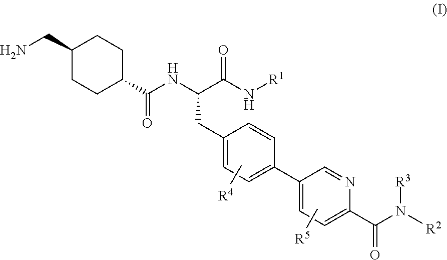
in which
R¹ is a group of the formula 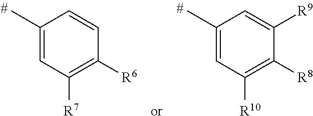
where # is the attachment site to the nitrogen atom,
R⁶ is 5-membered heteroaryl,
where heteroaryl may be substituted by a substituent selected from the group consisting of oxo, chlorine, cyano, hydroxyl and C₁-C₃-alkyl,
in which alkyl may be substituted by 1 to 3 substituents selected independently from the group consisting of hydroxyl, amino, hydroxycarbonyl and methoxy, or
in which alkyl may be substituted by 1 to 7 fluorine substituents, or
in which alkyl is substituted by a substituent selected from the group consisting of hydroxyl, amino, hydroxycarbonyl and methoxy, and in which alkyl is additionally substituted by 1 to 6 fluorine substituents,
R⁷ is hydrogen, fluorine or chlorine,
R⁸ and R⁹ together with the carbon atoms to which they are bonded form a 5-membered heterocycle,
where the heterocycle may be substituted by 1 to 2 substituents selected independently from the group consisting of oxo, chlorine, cyano, hydroxyl, C₁-C₃-alkyl, pyrazolyl and pyridyl,
in which alkyl may be substituted by 1 to 3 substituents selected independently from the group consisting of hydroxyl, amino, hydroxycarbonyl and methoxy, or
in which alkyl may be substituted by 1 to 7 fluorine substituents, or
in which alkyl is substituted by a substituent selected from the group consisting of hydroxyl, amino, hydroxycarbonyl and methoxy, and in which alkyl is additionally substituted by 1 to 6 fluorine substituents,
R₁₀ is hydrogen, fluorine or chlorine,
R² is hydrogen, C₁-C₆-alkyl, C₃-C₆-cycloalkyl, 4- to 9-membered heterocyclyl bonded via a carbon atom or 5- or 6-membered heteroaryl,
where alkyl may be substituted by 1 to 2 substituents selected independently from the group consisting of fluorine, hydroxyl, amino, hydroxycarbonyl, C₁-C₃-alkylamino, difluoromethyl, trifluoromethyl, —(OCH₂CH₂)ₙ—OCH₃, —(OCH₂CH₂)ₘ—OH, trimethylaminium and pyrrolidinyl,
in which n is a number from 1 to 6,
in which m is a number from 1 to 6, and
where cycloalkyl may be substituted by 1 to 2 substituents selected independently from the group consisting of oxo, fluorine, hydroxyl, amino, C₁-C₄-alkyl, C₁-C₃-alkylamino and morpholinyl,
in which alkyl and alkylamino may be substituted by 1 to 5 fluorine substituents, and
where heterocyclyl may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
where alkyl and alkylamino may be substituted by 1 to 5 substituents selected independently from the group consisting of hydroxyl and fluorine, and
where heterocyclyl may additionally be substituted by 1 to 4 substituents selected independently from the group consisting of fluorine and methyl, and
where heteroaryl may be substituted by 1 to 2 substituents selected independently from the group consisting of oxo, chlorine, cyano, hydroxyl and C₁-C₃-alkyl,
R³ is hydrogen or C₁-C₃-alkyl, or
R² and R³ together with the nitrogen atom to which they are bonded form a 4- to 7-membered heterocycle,
where the heterocycle may be substituted by 1 to 2 substituents selected independently from the group consisting of oxo, fluorine, hydroxyl, amino, hydroxycarbonyl, C₁-C₄-alkyl, C₁-C₃-alkylamino, difluoromethyl, trifluoromethyl, 2,2,2-trifluoroeth-1-yl, C₁-C₄-alkoxycarbonyl, aminocarbonyl and C₁-C₃-alkylaminocarbonyl,
R⁴ is hydrogen, fluorine, chlorine, methyl or methoxy,
R⁵ is hydrogen, fluorine, chlorine, C₁-C₄-alkyl, methoxy or trifluoromethyl,
or one of the salts thereof, solvates thereof or solvates of the salts thereof.

2. The compound of claim 1, characterized in that
R¹ is a group of the formula 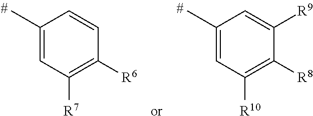
where # is the attachment site to the nitrogen atom,
R⁶ is 5-membered heteroaryl,
R⁷ is hydrogen,
R⁸ and R⁹ together with the carbon atoms to which they are bonded form a 5-membered heterocycle,
where the heterocycle may be substituted by an oxo substituent,
R₁₀ is hydrogen,
R² is C₁-C₆-alkyl, cyclopropyl, cyclobutyl, cyclohexyl, 4- to 9-membered heterocyclyl bonded via a carbon atom or 5- or 6-membered heteroaryl,
where alkyl may be substituted by a substituent selected from the group consisting of hydroxyl, C₁-C₃-alkylamino and trifluoromethyl, and
where cyclohexyl may be substituted by a substituent selected from the group consisting of hydroxyl, amino, C₁-C₃-alkylamino and morpholinyl, and
where heterocyclyl may be substituted by 1 to 2 substituents selected independently from the group consisting of oxo, fluorine and methyl, and
where heteroaryl may be substituted by 1 to 2 methyl substituents,
R³ is hydrogen, or
R² and R³ together with the nitrogen atom to which they are bonded form a 4- to 7-membered heterocycle,
where the heterocycle may be substituted by 1 to 2 substituents selected independently from the group consisting of oxo and methyl,
R⁴ is hydrogen,
R⁵ is methyl or trifloromethyl,
or one of the salts thereof, solvates thereof or solvates of the salts thereof.

3. The compound of claim 1, characterized in that
R¹ is a group of the formula 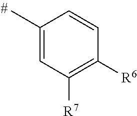
where # is the attachment site to the nitrogen atom,
R⁶ is tetrazolyl,
R⁷ is hydrogen, or
R¹ is 2,3-dihydro-1H-benzimidazol-5-yl or 2,3-dihydro-1H-indazol-6-yl,
where 2,3-dihydro-1H-benzimidazol-5-yl and 2,3-dihydro-1H-indazol-6-yl may be substituted by an oxo substituent,
R² is C₁-C₆-alkyl, cyclopropyl, cyclobutyl, cyclohexyl, heterocyclyl bonded via a carbon atom and selected from the group consisting of pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl, or pyrazolyl,
where alkyl may be substituted by a substituent selected from the group consisting of hydroxyl, C₁-C₃-alkylamino and trifluoromethyl, and
where cyclohexyl may be substituted by a substituent selected from the group consisting of hydroxyl, amino, C₁-C₃-alkylamino and morpholinyl, and
where pyrrolidinyl, piperidinyl, 3-azabicyclo[3.1.0]hex-6-yl, 8-azabicyclo[3.2.1]oct-3-yl and 3-oxa-9-azabicyclo[3.3.1]non-7-yl may be substituted by 1 to 2 substituents selected independently from the group consisting of oxo, fluorine and methyl, and
where pyrazolyl may be substituted by 1 to 2 methyl substituents,
R³ is hydrogen, or
R² and R³ together with the nitrogen atom to which they are bonded form a piperazinyl,
where piperazinyl may be substituted by 1 to 2 substituents selected independently from the group consisting of oxo and methyl,
R⁴ is hydrogen,
R⁵ is methyl or trifloromethyl,
or one of the salts thereof, solvates thereof or solvates of the salts thereof.

4. The compound of claim 1, characterized in that
R¹ is 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl or 1H-indazol-6-yl,
where the 5-membered heterocycle in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by an oxo substituent, and
where the benzyl ring in 2,3-dihydro-1H-benzimidazol-5-yl, 2,3-dihydro-1,3-benzoxazol-5-yl, 1H-benzimidazol-5-yl, 2,3-dihydro-1H-indazol-6-yl, 2,3-dihydro-1,3-benzoxazol-6-yl, 1H-benzimidazol-6-yl and 1H-indazol-6-yl may be substituted by a chlorine substituent,
R² is ethyl, isopropyl, cyclopropyl, cyclobutyl, cyclohexyl, or heterocyclyl bonded via a carbon atom, selected from the group of pyrrolidinyl and piperidinyl,
where ethyl is substituted by a trifluoromethyl substituent, and
where cyclohexyl is substituted by a substituent selected from the group consisting of hydroxyl, amino and C₁-C₃-alkylamino, and
where pyrrolidinyl and piperidinyl may be substituted by 1 to 2 substituents independently selected from the group consisting of oxo, fluorine and C₁-C₄-alkyl,
R³ is hydrogen,
R⁴ is hydrogen or fluorine,
R⁵ is methyl,
or one of the salts thereof, solvates thereof or solvates of the salts thereof.

5. The compound of claim 1, characterized in that
R¹ is 2,3-dihydro-1H-benzimidazol-5-yl, 2,3-dihydro-1H-indazol-6-yl or 1H-indazol-6-yl,
where the 5-membered heterocycle in 2,3-dihydro-1H-benzimidazol-5-yl, 2,3-dihydro-1H-indazol-6-yl and 1H-indazol-6-yl may be substituted by an oxo substituent, and
where the benzyl ring in 2,3-dihydro-1H-benzimidazol-5-yl may be substituted by a chlorine substituent,
R² is ethyl, isopropyl, cyclopropyl or cyclobutyl,
where ethyl is substituted by a trifluoromethyl substituent,
R³ is hydrogen,
R⁴ is hydrogen or fluorine,
R⁵ is methyl,
or one of the salts thereof, solvates thereof or solvates of the salts thereof.

6. A method of making the compound of claim 1 of the formula (I) or one of the salts thereof, solvates thereof or solvates of the salts thereof, characterized in that a compound of the formula 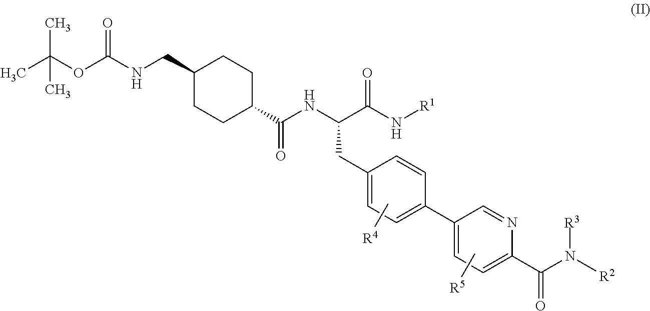
in which R¹, R², R³, R⁴ and R⁵ are each as defined in claim 1, is reacted with an acid.

7. A method for treatment and/or prophylaxis of diseases using the compound of claim 1.

8. A method of making a medicament for treatment and/or prophylaxis of diseases using the compound of claim 1.

9. A method of making a medicament for the treatment and/or prophylaxis of thrombotic or thromboembolic disorders or of severe perioperative blood loss using the compound of claim 1.

10. A medicament comprising the compound of claim 1 in combination with an inert, nontoxic, pharmaceutically suitable excipient.

11. A method for treatment and/or prophylaxis of thrombotic or thromboembolic disorders or severe perioperative blood loss using the medicament of claim 10.

12. A method for treating thrombotic or thromboembolic disorders or severe perioperative blood loss in man and animals by administration of a therapeutically effective amount of the compound of claim 1.

13. A method for treating thrombotic or thromboembolic disorders or severe perioperative blood loss in man and animals by administration of a therapeutically effective amount of the medicament of claim 10.

14. A method for treating thrombotic or thromboembolic disorders or severe perioperative blood loss in man and animals by administration of a therapeutically effective amount of the medicament of claim 8.
